(19) United States
(12) Patent Application Publication
(10) Pub. No.: US 2020/0392242 A1
(43) Pub. Date: Dec. 17, 2020

(54) Combination Therapies Comprising Daratumumab, Bortezomib, Thalidomide and Dexamethasone and Their Uses

(71) Applicant: Janssen Biotech, Inc., Horsham, PA (US)

(72) Inventors: Xiangyang Liu, Bridgewater, NJ (US); Jordan Schecter, Livingston, NJ (US)

(21) Appl. No.: 17/005,734

(22) Filed: Aug. 28, 2020

Related U.S. Application Data
(63) Continuation-in-part of application No. 16/850,360, filed on Apr. 16, 2020.
(60) Provisional application No. 62/836,361, filed on Apr. 19, 2019.
(60) Provisional application No. 62/836,408, filed on Apr. 19, 2019.
(60) Provisional application No. 62/836,445, filed on Apr. 19, 2019.
(60) Provisional application No. 62/836,557, filed on Apr. 19, 2019.

(51) Int. Cl. C07K 16/28; A61P 35/00; C12N 5/0775

(52) U.S. Cl. CPC C07K 16/2896; C07K 2317/565; C12N 5/0662; A61P 35/00

(57) ABSTRACT

Disclosed herein are combination therapies comprising daratumumab and their uses.

### CROSS-REFERENCE TO RELATED APPLICATIONS

This application is a Continuation-in-part of U.S. patent application Ser. No. 16/850,360, filed 16 Apr. 2020, currently pending, which claims priority to U.S. Provisional Application Ser. No. 62/836,361, filed 19 Apr. 2019, 62/836,408, filed 19 Apr. 2019, 62/836,445, filed 19 Apr. 2019 and 62/836,557, filed 19 Apr. 2019. The entire contents of each of the aforementioned applications is incorporated herein by reference in their entireties.

### FIELD OF THE INVENTION

Disclosed herein are combination therapies comprising daratumumab and their uses.

### SEQUENCE LISTING

This application contains a Sequence Listing submitted via EFS-Web, the entire content of which is incorporated herein by reference. The ASCII text file, created on 28 Aug. 2020, is named JBI6078USCIP1Seqlist.txt and is 13 kilobytes in size.

### BACKGROUND OF THE INVENTION

Multiple myeloma is a malignant disorder of the plasma cells, characterized by uncontrolled and progressive proliferation of a plasma cell clone. The disease leads to progressive morbidity and eventual mortality by lowering resistance to infection and causing significant skeletal destruction (with bone pain, pathological fractures, and hypercalcemia), anaemia, renal failure, neurological complications and hyperviscosity syndrome.

Multiple myeloma remains incurable with standard chemotherapy, despite the availability of multi-agent therapies.

There remains a need for new therapeutic options for the frontline setting that can better control the disease and provide deeper, more sustained responses and better long-term outcomes, including maintenance of health-related quality of life.

### SUMMARY OF THE INVENTION

The disclosure provides a method of treating a subject with newly diagnosed multiple myeloma, comprising:

providing a healthcare professional (HCP) daratumumab;
providing the HCP information that a combination therapy comprising daratumumab, bortezomib, thalidomide and dexamethasone (DVTd) is demonstrated to increase a likelihood of achieving a stringent complete response (sCR) or better in subjects with newly diagnosed multiple myeloma; wherein performing the steps a) and b) results in the subject with newly diagnosed multiple myeloma to receive combination therapy comprising DVTd by the HCP or by self-administration as instructed by the HCP, thereby treating the subject having the newly diagnosed multiple myeloma.

### BRIEF DESCRIPTION OF THE DRAWINGS

The summary, as well as the following detailed description, is further understood when read in conjunction with the appended drawings. For the purpose of illustrating the disclosed methods, the drawings show exemplary embodiments of the methods; however, the methods are not limited to the specific embodiments disclosed. In the drawings:

FIG. 1 shows the CASSIOPEIA study design.

FIG. 2 shows the results of the Kaplan-Meier estimates of progression-free survival among patients in the intention-to-treat population. The DARZALEX® (daratumumab) group received treatment with DARZALEX® (daratumumab), bortezomib, thalidomide and dexamethasone; the control group received treatment with bortezomib, thalidomide and dexamethasone.

FIG. 3 shows the results of the Kaplan-Meier estimates of the progression-free survival among patients with newly diagnosed multiple myeloma ineligible for autologous stem cell transplant. The DARZALEX (daratumumab) group received treatment with DARZALEX (daratumumab) in combination with lenalidomide and low-dose dexamethasone and the second at-risk group received treatment with lenalidomide and low-dose dexamethasone.

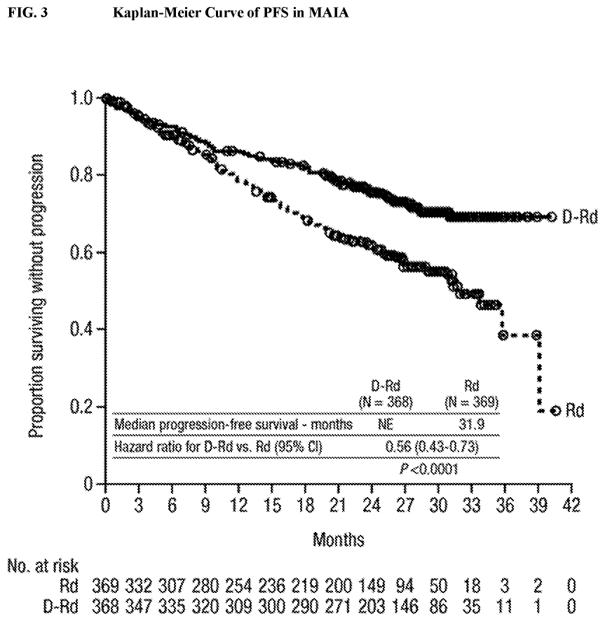

FIG. 4 shows the results of the Kaplan-Meier estimates of the progression-free survival among patients with newly diagnosed multiple myeloma ineligible for autologous stem cell transplant. The DARZALEX (daratumumab) group received treatment with DARZALEX (daratumumab) in combination with bortezomib, melphalan and prednisone, and the second at-risk group received treatment with bortezomib, melphalan and prednisone.

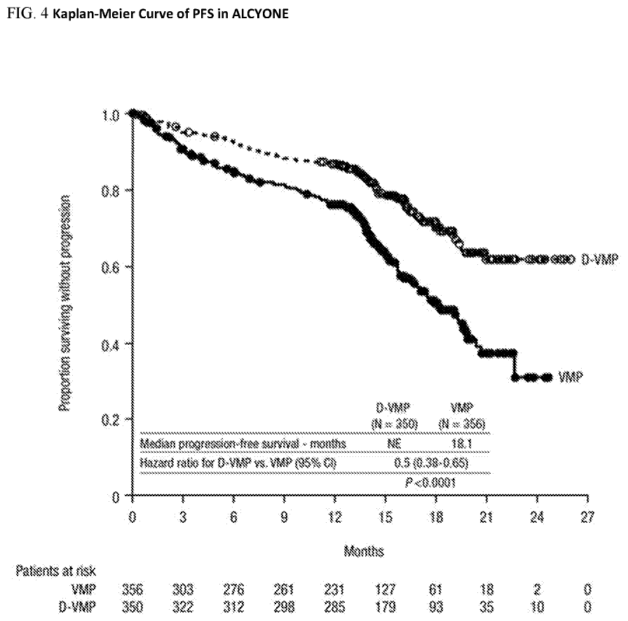

FIG. 5 shows the results of the Kaplan-Meier estimates of the progression-free survival among patients with newly diagnosed multiple myeloma eligible for autologous stem cell transplant. The DARZALEX (daratumumab) group received treatment with DARZALEX (daratumumab) in combination with bortezomib, thalidomide and dexamethasone, and the second at-risk group received treatment with bortezomib, thalidomide and dexamethasone.

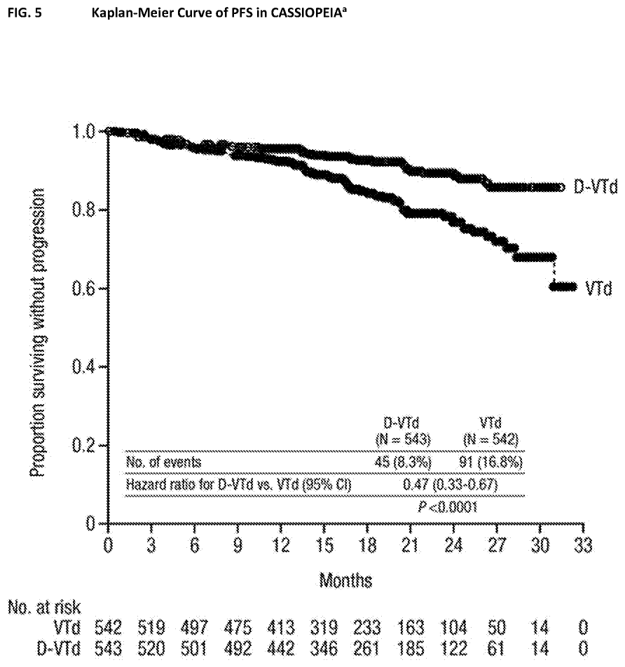

FIG. 6 shows the results of the Kaplan-Meier estimates of the progression-free survival among patients with multiple myeloma who had received at least one prior therapy. The DARZALEX (daratumumab) group received treatment with DARZALEX (daratumumab) in combination with lenalidomide and low-dose dexamethasone, and the second at-risk group received treatment with lenalidomide and low-dose dexamethasone.

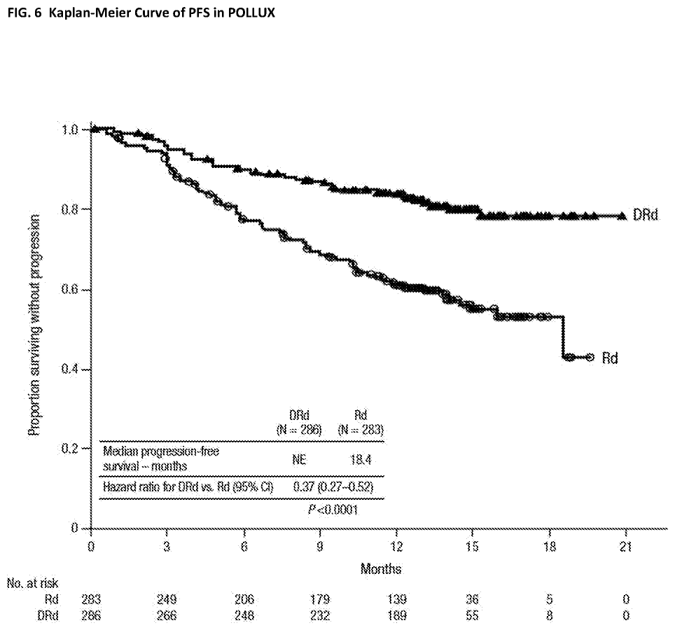

FIG. 7 shows the results of the Kaplan-Meier estimates of the progression-free survival among patients with multiple myeloma who had received at least one prior therapy. The DARZALEX (daratumumab) group received treatment with DARZALEX (daratumumab) in combination with bortezomib and dexamethasone and the second at-risk group received treatment with bortezomib and dexamethasone.

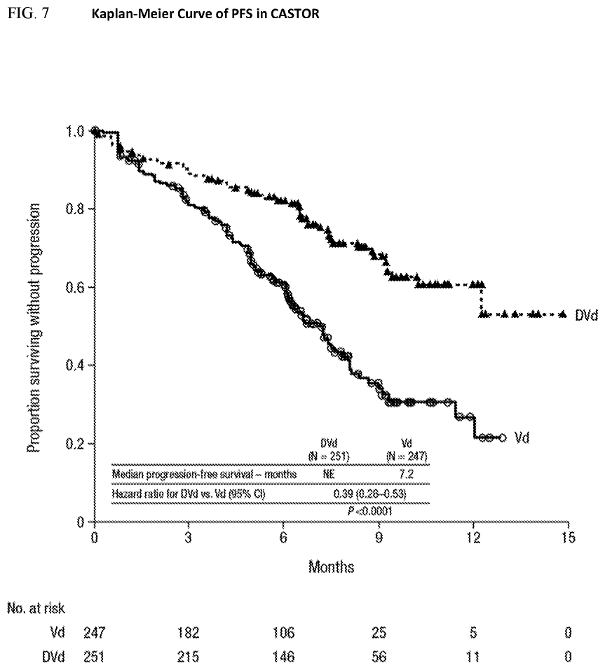

### DETAILED DESCRIPTION OF THE INVENTION

All publications, including but not limited to patents and patent applications, cited in this specification are herein incorporated by reference as though fully set forth.

It is to be appreciated that certain features of the invention which are, for clarity, described herein in the context of separate embodiments may also be provided in combination in a single embodiment. That is, unless obviously incompatible or specifically excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. Finally, although an embodiment may be described as part of a series of steps or part of a more general structure, each said step may also be considered an independent embodiment in itself, combinable with others.

### Definitions

When a list is presented, unless stated otherwise, it is to be understood that each individual element of that list, and every combination of that list, is a separate embodiment. For example, a list of embodiments presented as “A, B, or C” is to be interpreted as including the embodiments, “A,” “B,” “C,” “A or B,” “A or C,” “B or C,” or “A, B, or C.”

“About” means within an acceptable error range for the particular value as determined by one of ordinary skill in the art, which will depend in part on how the value is measured or determined, i.e., the limitations of the measurement system. Unless explicitly stated otherwise within the Examples or elsewhere in the Specification in the context of a particular assay, result or embodiment, “about” means within one standard deviation per the practice in the art, or a range of up to 5%, whichever is larger.

“About once a week” refers to an approximate number, and can include every 7 days±two days, i.e., every 5 days to every 9 days. The dosing frequency of “once a week” thus can be every five days, every six days, every seven days, every eight days, or every nine days.

“About once in two weeks” refers to an approximate number, and can include every 14 days±two days, i.e., every 12 days to every 16 days.

“About once in three weeks” refers to an approximate number, and can include every 21 days±two days, i.e., every 19 to every 23 days.

“About once in four weeks” refers to an approximate number, and can include every 28 days±two days, i.e., every 26 to every 30 days.

“About once in five weeks” refers to an approximate number, and can include every 35 days±two days, i.e., every 33 to every 37 days.

“About once in six weeks” refers to an approximate number, and can include every 42 days±two days, i.e., every 40 to every 38 days.

“About twice a week” refers to an approximate number, can include twice in one week, e.g., a first dose on day 1 and a second dose on day 2, day 3, day 4, day 5, day 6 or day 7 of the week, the first dose on day 2 and the second dose on day 3, day 4, day 5, day 6 or day 7 of the week, the first dose on day 3 and the second dose on day 4, day 5, day 6 or day 7 of the week, the first dose on day 4 and the second dose on day 5, day 6 or day 7 of the week, the first dose on day 5 and the second dose on day 6 or day 7 of the week, the first dose on day 6 and the second dose on day 7 of the week.

“Adverse event” or “AE” refers to any untoward medical occurrence in a clinical study subject administered an antibody that specifically binds CD38, such as daratumumab. An AE does not necessarily have a causal relationship with the treatment. An AE can therefore be any unfavorable and unintended sign (including an abnormal finding), symptom, or disease temporally associated with the use of a medicinal (investigational or non-investigational) product, whether or not related to the antibody that specifically binds CD38, such as daratumumab.

The conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

“Antibody” includes immunoglobulin molecules belonging to any class, IgA, IgD, IgE, IgG and IgM, or sub-class IgA1, IgA2, IgG1, IgG2, IgG3 and IgG4 and including either kappa (κ) and lambda (λ) light chain. Antibodies include monoclonal antibodies including human, humanized and chimeric monoclonal antibodies. Full-length antibody molecules are comprised of two heavy chains (HC) and two light chains (LC) inter-connected by disulfide bonds. Each heavy chain is comprised of a heavy chain variable region (VH) and a heavy chain constant region (comprised of domains CH1, hinge, CH2 and CH3). Each light chain is comprised of a light chain variable region (VL) and a light chain constant region (CL). The VH and the VL regions may be further subdivided into regions of hypervariability, termed complementarity determining regions (CDRs), interspersed with framework regions (FR). Each VH and VL is composed of three CDRs and four FR segments, arranged from amino-to-carboxy-terminus in the following order: FR1, CDR1, FR2, CDR2, FR3, CDR3 and FR4.

“Bortezomib” is designated chemically as [(1R)-3-methyl-1-[[(2S)-1-oxo-3-phenyl-2-[(pyrazinylcarbonyl) amino]propyl]amino]butyl] boronic acid. Bortezomib has the chemical structure shown in Formula 1. Bortezomib can be provided as a mannitol boronic ester which, in reconstituted form, consists of the mannitol ester in equilibrium with its hydrolysis product, the monomeric boronic acid. Bortezomibs drug substance exists in its cyclic anhydride form as a trimeric boroxine. Bortezomib is marketed under the trade name VELCADE®.

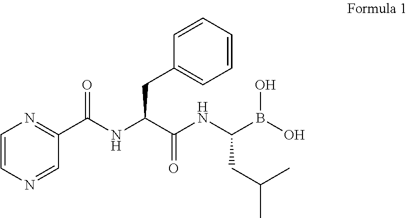 [SMILES: CC(C)C[C@H](NC(=O)[C@H](Cc1ccccc1)NC(=O)c1cnccn1)B(O)O]

“Biosimilar” (of an approved reference product/biological drug) refers to a biological product that is highly similar to the reference product notwithstanding minor differences in clinically inactive components with no clinically meaningful differences between the biosimilar and the reference product in terms of safety, purity and potency, based upon data derived from (a) analytical studies that demonstrate that the biological product is highly similar to the reference product notwithstanding minor differences in clinically inactive components; (b) animal studies (including the assessment of toxicity); and/or (c) a clinical study or studies (including the assessment of immunogenicity and pharmacokinetics or pharmacodynamics) that are sufficient to demonstrate safety, purity, and potency in one or more appropriate conditions of use for which the reference product is licensed and intended to be used and for which licensure is sought for the biosimilar. The biosimilar may be an interchangeable product that may be substituted for the reference product at the pharmacy without the intervention of the prescribing healthcare professional. To meet the additional standard of “interchangeability,” the biosimilar is to be expected to produce the same clinical result as the reference product in any given patient and, if the biosimilar is administered more than once to an individual, the risk in terms of safety or diminished efficacy of alternating or switching between the use of the biosimilar and the reference product is not greater than the risk of using the reference product without such alternation or switch. The biosimilar utilizes the same mechanisms of action for the proposed conditions of use to the extend the mechanisms are known for the reference product. The condition or conditions of use prescribed, recommended, or suggested in the labeling proposed for the biosimilar have been previously approved for the reference product. The route of administration, the dosage form, and/or the strength of the biosimilar are the same as those of the reference product and the biosimilar is manufactured, processed, packed or held in a facility that meets standards designed to assure that the biosimilar continues to be safe, pure and potent. The biosimilar may include minor modifications in the amino acid sequence when compared to the reference product, such as N- or C-terminal truncations that are not expected to change the biosimilar performance.

“Cancer” refers to an abnormal growth of cells which tend to proliferate in an uncontrolled way and, in some cases, to metastasize (spread) to other areas of a patient's body.

“CD38” refers to human cluster of differentiation 38 protein, a glycoprotein expressed on immune cells, including plasma cells, natural killer cells and sub-populations of B and T cells.

“Clinical efficacy endpoint” or “clinical endpoint” refers to an outcome that represents a clinical benefit, such as progression-free survival (PFS), time to disease progression (TTP), time to next treatment, overall response rate (ORR), proportion of subjects achieving partial response (PR), proportion of subjects achieving very good partial response (VGPR), proportion of subjects achieving complete response (CR), proportion of subjects achieving stringent complete response (sCR), proportion of subjects achieving a negative status for minimal residual disease (MRD), or proportion of subjects achieving both sCR and negative status for MRD.

“Clinically proven” refers to clinical efficacy results that are sufficient to meet approval standards of U.S. Food and Drug Administration (FDA), European Medicines Agency (EMA) or a corresponding national regulatory agency. For example, the clinical study may be an adequately sized, randomized, double-blinded controlled study used to clinically prove the effects of the drug.

“Co-administration,” “administration with,” “administration in combination with,” “in combination with” or the like, encompass administration of two or more therapeutics or drugs to a single patient, and are intended to include treatment regimens in which the therapeutics or drugs are administered by the same or different route of administration or at the same or different time.

“Combination” refers to a combination of two or more therapeutics or drugs that can be administered either together or separately.

“Complementarity determining regions” (CDRs) are “antigen binding sites” in an antibody. CDRs may be defined based on sequence variability (Wu and Kabat, J Exp Med 132:211-250, 1970; Kabat et al., Sequences of Proteins of Immunological Interest, 5th Ed. Public Health Service, National Institutes of Health, Bethesda, Md., 1991) or based on alternative delineations (see Lefranc et al., Dev Comparat Immunol 27:55-77, 2003). The International ImMunoGeneTics (IMGT) database (http://www_imgt_org) provides a standardized numbering and definition of antigen-binding sites.

“Complete response rate or better” (CR response rate or better) refers to the proportion of subjects achieving CR or stringent complete response (sCR) during or after the treatment.

“Comprising,” “consisting essentially of,” and “consisting of” are intended to connote their generally accepted meanings in the patent vernacular; that is, (i) “comprising,” which is synonymous with “including,” “containing,” or “characterized by,” is inclusive or open-ended and does not exclude additional, unrecited elements or method steps; (ii) “consisting of” excludes any element, step, or ingredient not specified in the claim; and (iii) “consisting essentially of” limits the scope of a claim to the specified materials or steps “and those that do not materially affect the basic and novel characteristics” of the claimed invention. Embodiments described in terms of the phrase “comprising” (or its equivalents) also provide as embodiments those independently described in terms of “consisting of” and “consisting essentially of.”

“Consolidation”, “consolidation therapy” or “consolidation period” refers to a short duration of treatment given to a subject after the subject has been treated with high dose chemotherapy (HDC) and autologous stem cell transplant (ASCT); i.e., post-HDC and ASCT. In the context of this disclosure, “consolidation therapy” refers to post-HDC and ASCT treatment with a combination of an antibody that specifically binds CD38, bortezomib, thalidomide and dexamethasone or with a combination of bortezomib, thalidomide and dexamethasone.

“Corticosteroid” refers to a class of steroid hormones that are produced in the adrenal cortex or produced synthetically refers to dexamethasone, methylprednisolone, prednisolone and prednisone. Dexamethasone is marketed under the trade name DECARON®.

“Cycle” refers to the administration schedule of one or more therapeutics or drugs and refers to the period of time when the one or more therapeutics or drugs is administered to a subject. Cycle may include days in which the drug is administered and periods of rest in which the drug is not administered. Cycle length may vary, and can be for example 2 weeks, 3 weeks, 28-days (or 4 weeks), 5 weeks or 6 weeks.

“Daily” in the context of dosing refers to a total dose of a drug such as lenalidomide administered to a subject in a day. The dose may be divided to two or more administrations during the day, or given as one administration per day. For example, the total dose may be 25 mg daily administered as a single dose.

“Daratumumab” refers to an antibody that specifically binds CD38 comprising a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5, a LCDR3 of SEQ ID NO: 6, a heavy chain variable region (VH) of SEQ ID NO: 7, a light chain variable region (VL) of SEQ ID NO: 8, a heavy chain (HC) of SEQ ID NO: 9 and a light chain (LC) of SEQ ID NO: 10. Daratumumab is marketed under the trade name DARZALEX®.

“Daratumumab” refers to any drug comprising daratumumab as an active ingredient, including biosimilars of DARZALEX®.

“Daratumumab-containing drug product” refers to any drug in which daratumumab is an active ingredient.

“Dexamethasone” is designated chemically as 9-fluoro-11β,17,21-trihydroxy-16α-methylpregna-1,4-diene-3,20-dione. The structure of dexamethasone is shown in Formula 2.

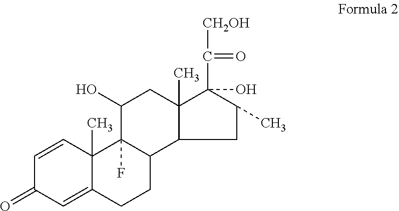

“Dose” refers to the amount or quantity of the therapeutic or the drug to be taken each time.

“Dosage” refers to the information of the amount of the therapeutic or the drug to be taken by the subject and the frequency of the number of times the therapeutic is to be taken by the subject.

“Drug product” (DP) refers to a finished dosage form, for example, a tablet, capsule or solution that contains an active pharmaceutical ingredient (e.g., drug substance), generally, but not necessarily, in association with inactive ingredients.

“Drug substance” (DS) refers to any substance or mixture of substances intended to be used in the manufacture of a drug (medicinal) product and that, when used in the production of a drug, becomes an active ingredient of the drug product. Such substances are intended to furnish pharmacological activity or other direct effect in the diagnosis, cure, mitigation, treatment, or prevention of disease or to affect the structure or function of the body.

“Duration of complete response” (duration of CR) refers to the time between the date of the initial documentation of CR to the date of the first documented evidence of relapse of CR or disease progression, whichever occurs first.

“Duration of response” refers to the time between the date of initial documentation of a response (partial response (PR) or better) to the date of the first documented evidence of progressive disease.

“Duration of stringent complete response” (duration of sCR) refers to the time between the date of the initial documentation of sCR to the date of the first documented evidence of relapse of sCR or disease progression, whichever occurs first.

“Effective” refers to a dose or dosage of a therapeutic or a drug (such as an antibody that specifically binds CD38) or a combination of therapeutics or drugs (such as a combination of an antibody that specifically binds CD38, bortezomib, thalidomide or dexamethasone) that provides a therapeutic effect for a given condition and administration regimen in a subject receiving or who has received the therapeutic or the drug or the combination of the therapeutics or drugs. “Effective” is intended to mean an amount sufficient to reduce and/or prevent a clinically significant deficit in the activity, function and response of the subject, or to cause an improvement in a clinically significant condition in the subject.

“Frontline” or “firstline” therapy refers to the first treatment of a disease, such as multiple myeloma, administered to the subject.

“Glutamic acid derivative” refers to immunomodulatory drugs that are derivatives of glutamic acid such as lenalidomide, thalidomide and pomalidomide. Lenalinomide is marketed under the trade name REVLIMID®. Thalidomide is marketed under the trade name THALOMID®. Pomalidomide is marketed under the trade name POMALYST®

“Healthcare professional” (HCP) refers to a medical doctor, a nurse, a nurse's assistant, or a person working under direct instructions by the medical doctor or the nurse, or any person working in a hospital or a place in which treatment can be provided to the subject.

“High dose chemotherapy” (HDC) and “autologous stem cell transplant” (ASCT) refer to the treatment of subjects with newly diagnosed multiple myeloma who are considered fit (e.g. subjects are “eligible”). Subjects under the age of 65 years who have one or more comorbidities likely to have a negative impact on tolerability of HDC and ASCT or subjects over the age of 65 years are usually not considered eligible for HDC and ASCT due to their frail physical status which increases the risk of mortality and transplant-related complications (e.g. subjects are “ineligible”). An exemplary comorbidity is a renal dysfunction. Exemplary HDC regimens are melphalan at a dose of 200 mg/m² with dose reductions based on age and renal function, cyclophosphamide and melphalan, carmustine, etoposide, cytarabine, and melphalan (BEAM), high-dose idarubicin, cyclophosphamide, thiotepa, busulfan, and cyclophosphamide, busulfan and melphalan, and high-dose lenalidomide (Mahajan et al., Ther Adv Hematol 9:123-133, 2018).

“High risk multiple myeloma” refers to multiple myeloma that is characterized by one or more cytogenetic abnormalities del17p, t(4;14), t(14;20), t(14;16) or del13, or any combination thereof.

“Induction”, “induction therapy” or “induction period” refers to the first treatment given for a disease with the intention of reducing the amount of malignant plasma cell burden and improving the depth of response. In the context of this disclosure, “induction therapy” refers to treatment with a combination of an antibody that specifically binds CD38, bortezomib, thalidomide and dexamethasone or a combination of bortezomib, thalidomide and dexamethasone prior to treatment with HDC and ASCT.

“Information” refers to reported results from clinical trials and can be provided in written or electronic form, or orally, or it can be available on internet.

“Infusion related reaction” (IRR) refers to any sign or symptom experienced by a subject during the administration of a drug or a therapeutic or any event occurring within 24-hours of administration. IRRs are typically classified as Grade 1, 2, 3 or 4.

“Label” and “labeling” are used interchangeably herein and refers to all labels and displays of written, printed, or graphic information on, in or accompanying a container or package comprising a drug, such as daratumumab, or otherwise available electronically or on internet. “Label” and “labeling” include package insert and prescribing information.

“Lenalidomide” a thalidomide analogue, is an immunomodulatory agent with antiangiogenic and antineoplastic properties. The chemical name is 3-(4-amino-1-oxo 1,3-dihydro-2H-isoindol-2-yl) piperidine-2,6-dione and it has the structure shown in Formula 3. Lenalinomide is marketed under the trade name REVLIMID®.

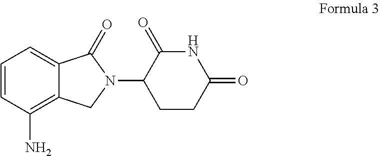 [SMILES: Nc1cccc2c1CN(C1CCC(=O)NC1=O)C2=O]

“Maintenance therapy” refers to the treatment given for a disease after remission or best response is achieved, in order to prevent or delay relapse. In the context of this disclosure, maintenance therapy refers to monotherapy with daratumumab after consolidation therapy.

“Minimal residual disease” (MRD) refers to a small number of clonal multiple myeloma cells that remain in the patient after treatment and/or during remission.

“MRD negative” or “negative status for MRD” refers to a ratio of 1:10×10⁵ or less clonal multiple myeloma cells in a bone marrow aspirate sample obtained from the subject.

“MRD negativity rate” refers to the proportion of subjects assessed as MRD negative at any timepoint after the date of randomization.

“Multiple myeloma” refers to a malignant disorder of plasma cells characterized by uncontrolled and progressive proliferation of one or more malignant plasma cells. The abnormal proliferation of plasma (myeloma) cells causes displacement of the normal bone marrow leading to dysfunction in hematopoietic tissue and destruction of the bone marrow architecture, resulting in progressive morbidity and eventual mortality.

“Newly diagnosed” refers to a human subject who has been diagnosed with but has not yet received treatment for multiple myeloma.

“Overall response rate” (ORR) refers to the proportion of subjects who achieve partial response (PR), very good partial response (VGPR), complete response (CR) or stringent complete response (sCR) during or after the treatment.

“Overall survival” (OS) is defined as the time from initiation of therapy to the date of death due to any cause. For the purpose of the clinical trial described in the example, OS is defined as the time from randomization of study population to the date of the patient's death.

“Percent w/v” (% w/v) refers to weight in grams per 100 m.

“Per week” refers to a total dose of a drug such as dexamethasone administered to a subject in one week. The dose may be divided to two or more administrations during the same day or different days. For example, the total dose may be 40 mg administered 20 mg on day 1 and 20 mg on day 3 of a week.

“Pharmaceutical combination” refers to a combination of two or more therapeutics or drugs administered either together or separately.

“Pharmaceutical composition” refers to a product that results from combining an antibody that specifically binds CD38 and a hyaluronidase as a fixed combination. “Fixed combinations” refers to a single pharmaceutical composition comprising the anti-CD38 antibody and the hyaluronidase administered simultaneously in the form of a single entity or dosage. Pharmaceutical composition typically includes a pharmaceutically acceptable carrier.

“Pharmaceutically acceptable carrier” or “excipient” refers to an ingredient in a pharmaceutical composition, other than the active ingredient, which is nontoxic to a subject. Pharmaceutically acceptable carrier includes, but is not limited to, a buffer, stabilizer or preservative.

“Post-ASCT and consolidation CR rate” refers to the proportion of subjects who have achieved CR or better by the end of consolidation therapy.

“Post-ASCT and consolidation MRD negative rate” refers to the proportion of subjects who have achieved MRD negative status by the end of consolidation therapy.

‘Post-consolidation” refers to treatment period ending at the end of consolidation therapy.

“Post-induction” refers to treatment period ending at the end of induction therapy.

“Post-induction stringent complete response rate” (post-induction sCR rate) refers to the proportion of subjects who have achieved sCR prior to HDC and ASCT.

“Post-induction overall response rate” (post-induction ORR) refers to the proportion of subjects who have achieved partial response (PR) or better by the end of induction.

“Post-induction very good partial response or better” (post-induction VGPR or better) refers to the proportion of subjects who have achieved VGPR, complete response (CR) or stringent complete response (sCR) by the end of induction.

“Progression-free survival” (PFS) means time from initiation of therapy to first evidence of disease progression or death due to any cause, whichever occurs first. For the purpose of the clinical trial described in the example, PFS is defined as the duration from the date of randomization of study population to the first documented progressive disease (PD) or death due to any cause, whichever occurs first.

“Progression-free survival 2” (PFS2) refers to the time from the second randomization to time of subsequent progression on next-line of therapy after disease progression on study treatment.

“Progressive disease” (PD), “stable disease” (SD), “partial response” (PR), “very good partial response” (VGPR), “complete response” (CR) and “stringent complete response” (sCR) refer to response to treatment and take their customary meanings as will be understood by a person skilled in the art of designing, conducting, or reviewing clinical trials. Response to treatment may be assessed using International Myeloma Working Group (IMWG) uniform response criteria recommendations (International Uniform Response Criteria Consensus Recommendations) as shown in Table 1.

“Refractory” refers to a disease that does not respond to a treatment. A refractory disease can be resistant to a treatment before or at the beginning of the treatment, or a refractory disease can become resistant during a treatment.

“Relapsed” refers to the return of a disease or the signs and symptoms of a disease after a period of improvement after prior treatment with a therapeutic.

“Reference product” refers to an approved biological product such as DARZALEX® brand of daratumumab against which a biosimilar product is compared. A reference product is approved in the U.S. based on, among other things, a full complement of safety and effectiveness data.

“Safe” as it relates to a composition, dose, dosage regimen, treatment or method with a therapeutic or a drug (such as an antibody that specifically binds CD38 or a combination of an antibody that specifically binds CD38, bortezomib, thalidomide and dexamethasone (D-VTD) refers to a favorable benefit:risk ratio with an acceptable frequency and/or acceptable severity of adverse events (AEs) and/or treatment-emergent adverse events (TEAEs) compared to the standard of care (such as for example a combination of lenalidomide and dexamethasone) or to another comparator.

“Safe and effective” refers to an amount and/or dosage of a drug (such as an antibody that specifically binds CD38) or a combination of drugs (such as a combination of an antibody that specifically binds CD38, bortezomib, thalidomide and dexamethasone (D-VTD)) that elicits the desired biological or medicinal response in a subject's biological system without the risks outweighing the benefits of such response in accordance with the Federal Food, Drug, and Cosmetic Act, as amended (secs. 201-902, 52 Stat. 1040 et seq., as amended; 21 U.S.C. §§ 321-392). Safety is evaluated in laboratory, animal and human clinical testing to determine the highest tolerable dose or the optimal dose of the drug or the combination of drugs needed to achieve the desired benefit. Efficacy is evaluated in human clinical trials and determining whether the drug or the combination of drugs demonstrates a health benefit over a placebo or other intervention. Safe and effective drugs or a combination of drugs are granted marketing approval by the FDA for their indicated use.

An antibody that “specifically binds CD38” refers to antibody binding CD38 with greater affinity than to other antigens. Typically, the antibody binds to CD38 with an equilibrium dissociation constant (K_{D}) of about 1×10⁻⁸ M or less, for example about 1×10⁻⁹M or less, about 1×10⁻¹⁰ M or less, about 1×10⁻¹¹M or less, or about 1×10⁻¹²M or less, typically with a K_{D} that is at least one hundred-fold less than its K_{D} for binding to a non-specific antigen (e.g., BSA, casein). The K_{D} may be measured using standard procedures. Antibodies that specifically bind CD38 may, however, have cross-reactivity to other related antigens, for example to the same antigen from other species (homologs), such as monkey, for example Macaca fascicularis (cynomolgus, cyno), Pan troglodytes (chimpanzee, chimp) or Callithrix jacchus (common marmoset, marmoset).

“Stringent complete response rate or better” (sCR rate or better) refers to the proportion of subjects achieving sCR during or after the treatment.

“Subject” refers to a human patient. The terms “subject” and “patient” can be used interchangeably herein.

“Thalidomide” is designated chemically as α-(N-phthalimido)glutarimide (CAS number 50-35-1). Thalidomide has the structure shown in Formula 4. Thalidomide is marketed under a trade name THALOMID®.

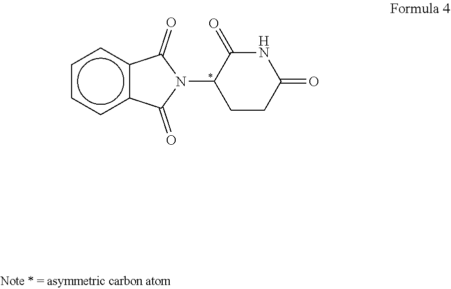

“Therapeutically effective amount” refers to an amount effective, at dosages and for periods of time necessary, to achieve the desired therapeutic result. A therapeutically effective amount may vary according to factors such as the disease state, age, sex, and weight of the individual, and the ability of a therapeutic or a combination of therapeutics to elicit a desired response in the individual. Exemplary indicators of an effective therapeutic or combination of therapeutics include, for example, improved well-being of the patient, reduction in a tumor burden, arrested or slowed growth of a tumor, and/or absence of metastasis of cancer cells to other locations in the body.

“Time to disease progression” (TTP) means time from the date of randomization to the date of confirmed progressive disease (PD) or death due to PD, whichever occurs first.

“Time to disease progression 2” (TTP2) refers to the time from the date of second randomization to confirmed progressive disease (PD) or death due to PD, whichever occurs first.

“Time to next treatment” refers to the time from randomization to the start of the next-line treatment.

“Time to response” refers to the time between the randomization and the first efficacy evaluation that the subject has met all criteria for partial response (PR) or better.

“Time to subsequent anti-myeloma therapy” refers to the time from the initiation of therapy to documentation of administration of a new anti-myeloma therapy to the subject.

“Treat”, “treating” or “treatment” refers to therapeutic treatment. Individuals in need of treatment include those subjects diagnosed with the disorder of a symptom of the disorder. Subject that may be treated also include those prone or susceptible to have the disorder, or those in which the disorder is to be prevented. Beneficial or desired clinical results include alleviation of symptoms, diminishment of extent of disease, stabilized (i.e., not worsening) state of disease, delay or slowing of disease progression, amelioration or palliation of the disease state, disease remission (whether partial or total) and prolonging survival as compared to expected survival if a subject was not receiving treatment or was receiving another treatment.

“Treatment emergent adverse events” (TEAE) as used herein takes its customary meaning as will be understood by a person skilled in the art of designing, conducting, or reviewing clinical trials and refers to an AE considered associated with the use of an antibody that specifically binds CD38 if the attribution is possible, probable, or very likely.

“Unacceptable adverse events” and “unacceptable adverse reaction” refers to all harm or undesired outcomes associated with or caused by administration of a pharmaceutical composition or a therapeutic, and the harm or undesired outcome reaches such a level of severity that a regulatory agency deems the pharmaceutical composition or the therapeutic unacceptable for the proposed use.

“Very good partial response or better” (VGPR rate or better) refers to the proportion of subjects achieving VGPR, complete response (CR) or stringent complete response (sCR) during or after the treatment.

### Multiple Myeloma

Multiple myeloma causes significant morbidity and mortality. It accounts for approximately 1% of all malignancies and 13% of hematologic cancers worldwide. Approximately 50,000 patients per year are diagnosed with multiple myeloma in the EU and US, and 30,000 patients per year die due to multiple myeloma.

The majority of patients with multiple myeloma produce a monoclonal protein (paraprotein, M-protein or M-component) which is an immunoglobulin (Ig) or a fragment of one that has lost its function (Kyle and Rajkumar, Leukemia 23:3-9, 2009; Palumbo and Anderson, N Engl J Med 364:1046-1060, 2011). Normal immunoglobulin levels are compromised in patients, leading to susceptibility of infections. The proliferating multiple myeloma cells displace the normal bone marrow leading to dysfunction in normal hematopoietic tissue and destruction of the normal bone marrow architecture, which is reflected by clinical findings such as anemia, paraprotein in serum or urine, and bone resorption seen as diffuse osteoporosis or lytic lesions shown in radiographs (Kyle et al., Mayo Clin Proc 78:21-33, 2003). Furthermore, hypercalcemia, renal insufficiency or failure, and neurological complications are frequently seen. A small minority of patients with multiple myeloma are non-secretory.

Treatment choices for multiple myeloma vary with age, comorbidity, the aggressiveness of the disease, and related prognostic factors (Palumbo and Anderson, N Engl J Med 364:1046-1060, 2011). Newly diagnosed patients with multiple myeloma are typically categorized into 2 subpopulations usually defined by their age and suitability for the subsequent approach to treatment. Younger patients will typically receive an induction regimen followed by consolidation treatment with high-dose chemotherapy (HDC) and autologous stem cell transplantation (ASCT). For those not considered suitable for HDC and ASCT, longer-term treatment with multi-agent combinations including alkylators, high-dose steroids, and novel agents are currently considered as standards of care. In general, patients over the age of 65 or with significant comorbidities are usually not considered eligible for HDC and ASCT. For many years, the oral combination melphalan-prednisone (MP) was considered the standard of care for patients with multiple myeloma who were not eligible for ASCT (Gay and Palumbo, Blood Reviews 25:65-73, 2011). The advent of immunomodulatory agents (IMiDs) and proteasome inhibitors (PIs) has led to a multiplicity of new treatment options for newly diagnosed patients not considered suitable for transplant-based therapy.

### Multiple Myeloma Diagnosis

Subjects afflicted with multiple myeloma satisfy the CRAB (calcium elevation, renal insufficiency, anemia and bone abnormalities) criteria, and have clonal bone marrow plasma cells ≥10% or biopsy-proven bony or extramedullary plasmacytoma, and measurable disease. Measurable disease is defined by any of the following;
IgG myeloma: Serum monoclonal paraprotein (M-protein) level ≥1.0 g/dL or urine M-protein level ≥200 mg/24 hours; or
IgA, IgM, IgD, or IgE multiple myeloma: serum M-protein level ≥0.5 g/dL or urine M-protein level ≥200 mg/24 hours; or
Light chain multiple myeloma without measurable disease in serum or urine: Serum immunoglobulin free light chain ≥10 mg/dL and abnormal serum immunoglobulin kappa lambda free light chain ratio.

### CRAB Criteria

Hypercalcemia: serum calcium >0.25 mM/L (>1 mg/dL) higher than the upper limit of the normal range [ULN] or >2.75 mM/L (>11 mg/dL)
Renal insufficiency: creatinine clearance <40 mL/min or serum creatinine >177 μM/L (>2 mg/dL)
Anemia: hemoglobin >2 g/dL below the lower limit of normal or hemoglobin <10 g/dL
Bone lesions: one or more osteolytic lesions on skeletal radiography, CT, or PET-CT

Response to treatment may be assessed using International Myeloma Working Group (IMWG) uniform response criteria recommendations (International Uniform Response Criteria Consensus Recommendations) as shown in Table 1.

TABLE 1

| Response | Response Criteria |
|---|---|
| Stringent | CR as defined below, plus |
| complete | Normal FLC ratio, and |
| Response (sCR) | Absence of clonal PCs by immunohistochemistry, immunofluorescence or 2-to |
| | 4-color flow cytometry |
| Complete | Negative immunofixation on the serum and urine, and |
| response (CR) | Disappearance of any soft tissue plasmacytomas, and |
| | <5% PCs in bone marrow |
| Very good | Serum and urine M-component detectable by immunofixation but not on |
| partial | electrophoresis, |
| Response | or |
| (VGPR) | ≥90% reduction in serum M-protein plus urine M-protein <100 mg/24 hours |
| Partial response | ≥50% reduction of serum M-protein and reduction in 24-hour urinary M-protein |
| (PR) | by ≥90% or to <200 mg/24 hours |
| | If the serum and urine M-protein are not measurable, a decrease of ≥50% in the |
| | difference between involved and uninvolved FLC levels is required in place of |
| | the M-protein criteria |
| | If serum and urine M-protein are not measurable, and serum free light assay is |
| | also not measurable, ≥50% reduction in bone marrow PCs is required in place of |
| | M-protein, provided baseline bone marrow plasma cell percentage was ≥30% |
| | In addition to the above criteria, if present at baseline, a ≥50% reduction in the |
| | size of soft tissue plasmacytomas is also required. |
| Stable disease | Not meeting criteria for CR, VGPR, PR, or progressive disease |
| (SD) | |
| Progressive | Increase of 25% from lowest response value in any one of the following: |
| disease (PD) | Serum M-component (absolute increase must be ≥0.5 g/dL), |
| | Urine M-component (absolute increase must be ≥200 mg/24 hours), |
| | Only in subjects without measurable serum and urine M-protein levels: the |
| | difference between involved and uninvolved FLC levels (absolute increase must |
| | be >10 mg/dL) |
| | Only in subjects without measurable serum and urine M-protein levels and |
| | without measurable disease by FLC levels, bone marrow PC percentage |
| | (absolute percentage must be ≥10%) |
| | Bone marrow plasma cell percentage: the absolute percentage must be >10% |
| | Definite development of new bone lesions or soft tissue plasmacytomas or |
| | definite increase in the size of existing bone lesions or soft tissue plasmacytomas |
| | Development of hypercalcemia (corrected serum calcium >11.5 mg/dL) that can |
| | be attributed solely to the PC proliferative disorder |

EBMT = European Group for Blood and Marrow Transplantation;
FLC = free light chain;
PC = plasma cell

### Methods of Treatment and Uses

### Method of the Disclosure

The disclosure provides a method of treating a subject with newly diagnosed multiple myeloma, comprising administering to the subject a safe and effective combination therapy comprising daratumumab, bortezomib, thalidomide and dexamethasone.

The disclosure also provides a method of treating a subject with newly diagnosed multiple myeloma, comprising administering to the subject a safe and effective combination therapy demonstrated to increase a likelihood of achieving a stringent complete response (sCR) or better in subjects with newly diagnosed multiple myeloma, wherein the safe and effective combination therapy comprises daratumumab, bortezomib, thalidomide and dexamethasone.

In some embodiments, the likelihood of achieving the sCR or better is about 28% or higher.

The disclosure also provides a method of treating a subject with newly diagnosed multiple myeloma, comprising administering to the subject a safe and effective combination therapy demonstrated to increase a likelihood of achieving a complete response (CR) or better in subjects with newly diagnosed multiple myeloma, wherein the safe and effective combination therapy comprises daratumumab, bortezomib, thalidomide and dexamethasone.

In some embodiments, the likelihood of achieving the CR or better is about 38% or higher.

The disclosure also provides a method of treating a subject with newly diagnosed multiple myeloma, comprising administering to the subject a safe and effective combination therapy demonstrated to increase a likelihood of achieving a negative status for minimal residual disease (MRD) in subjects with newly diagnosed multiple myeloma, wherein the safe and effective combination therapy comprises daratumumab, bortezomib, thalidomide and dexamethasone.

In some embodiments, the likelihood of achieving the negative status for MRD is about 33% or higher.

MRD status may be assessed from bone marrow aspirate samples using for example next generation sequencing (NGS) of immunoglobulin heavy and light chains. The updated, analytically validated version of the clonoSEQ® Assay (Version 2) by Adaptive Biotechnologies may be used for the detection, quantification and analysis of MRD.

The disclosure also provides a method of treating a subject with newly diagnosed multiple myeloma, comprising administering to the subject a safe and effective combination therapy demonstrated to reduce a risk of progression of multiple myeloma or death in subjects with newly diagnosed multiple myeloma, wherein the safe and effective combination therapy comprises daratumumab, bortezomib, thalidomide and dexamethasone.

In some embodiments, the risk of progression of multiple myeloma or death is reduced by about 53%.

In some embodiments, the subject with newly diagnosed multiple myeloma is eligible for autologous stem cell transplant (ASCT). In eligible subjects, ASCT is provided in conjunction with high dose chemotherapy (HDC) as described herein.

In some embodiments, the safe and effective combination therapy comprises about 16 mg/kg daratumumab, about 1.3 mg/m² bortezomib, about 100 mg thalidomide and between about 20 mg and about 40 mg dexamethasone.

In some embodiments, the method comprises an induction phase, a high dose chemotherapy (HDC) and autologous stem cell transplant (ASCT), and a consolidation phase.

In some embodiments, the induction phase comprises four 28-day induction cycles comprising about 16 mg/kg daratumumab administered once a week on weeks 1 to 8 and once in two weeks on weeks 9-16;

about 1.3 mg/m² bortezomib administered twice a week on week 1 and week 2 in the four 28-day induction cycles;
about 100 mg thalidomide daily; and
about 40 mg dexamethasone administered twice a week on week 1, week 2 and week 3 in the first and the second 28-day induction cycle, about 40 mg twice a week on week 1 and about 20 mg twice a week on week 2 and 3 in the third and the fourth 28-day induction cycle.

In some embodiments, the induction phase comprises four 28-day induction cycles comprising about 16 mg/kg daratumumab administered once a week on weeks 1 to 8 and once in two weeks on weeks 9-16;

about 1.3 mg/m² bortezomib administered on days 1, 4, 8 and 11 in the four 28-day induction cycle;
about 100 mg thalidomide daily; and
about 40 mg dexamethasone administered on days 1, 2, 8, 9, 15, 16 in the first and the second 28-day induction cycle, about 40 mg on days 1 and 2 and about 20 mg on days 8, 9, 15 and 16 in the third and the fourth 28-day induction cycle.

In some embodiments, the induction phase is followed by the HDC and ASCT.

In some embodiments, the HDC comprises melphalan.

In some embodiments, melphalan is administered at a dose of about 200 mg/m², optionally over a period of 24 to 48 hours.

In some embodiments, the HDC and ASCT is followed by the consolidation phase.

In some embodiments, the consolidation phase comprises two 28-day consolidation cycles comprising

about 16 mg/kg daratumumab administered once in two weeks on weeks 1 to 8;
about 1.3 mg/m² bortezomib administered twice a week on week 1 and week 2 in each two 28-day consolidation cycle;
about 100 mg thalidomide daily; and
about 20 mg dexamethasone administered twice a week on week 1, week 2 and week 3 in each two 28-day consolidation cycle.

In some embodiments, the consolidation phase comprises two 28-day consolidation cycles of

about 16 mg/kg daratumumab on days 1 and 15 in each two 28-day consolidation cycle;
about 1.3 mg/m² bortezomib on days 1, 4, 8 and 11 in each two 28-day consolidation cycles;
about 100 mg thalidomide daily; and
about 20 mg dexamethasone on days 1, 2, 8, 9, 15 and 16 in each two 28-day consolidation cycles.

In some embodiments, dexamethasone is administered as pre-medication on daratumumab administration days.

In some embodiments, daratumumab is administered intravenously, bortezomib is administered subcutaneously or intravenously, thalidomide is administered orally and dexamethasone is administered intravenously or orally.

In some embodiments, thalidomide, dexamethasone or both thalidomide and dexamethasone are self-administered.

In some embodiments, daratumumab is provided for administration by a manufacturer of daratumumab in a single-dose vial comprising 100 mg daratumumab in 5 mL of solution or in a single-dose vial comprising 400 mg daratumumab in 20 mL of solution.

In some embodiments, each single-dose vial comprising 100 mg daratumumab in 5 mL of solution and each single-dose vial comprising 400 mg daratumumab in 20 mL of solution further comprises glacial acetic acid, mannitol, polysorbate 20, sodium acetate trihydrate and sodium chloride.

In some embodiments, each single-dose vial comprising 100 mg daratumumab in 5 mL of solution contains 0.9 mg glacial acetic acid, 127.5 mg mannitol, 2 mg polysorbate 20, 14.8 mg sodium acetate trihydrate, 17.5 mg sodium chloride and water for injection, and each single-dose vial comprising 400 mg daratumumab in 20 mL of solution contains 400 mg daratumumab, 3.7 mg glacial acetic acid, 510 mg mannitol, 8 mg polysorbate 20, 59.3 mg sodium acetate trihydrate, 70.1 mg sodium chloride and water for injection.

In some embodiments, daratumumab is diluted into 0.9% sodium chloride prior to administration.

In some embodiments, information that a combination therapy comprising daratumumab, bortezomib, thalidomide and dexamethasone is safe and effective is provided on a daratumumab-containing drug product label or package insert.

Exemplary information is clinical trial results from an open-label, randomized active-controlled phase 3 study CASSIOPEIA, listed at ClinicalTrials_gov database as study NCT02541383.

In some embodiments, the daratumumab-containing drug product label includes information that a recommended dose of daratumumab is 16 mg/kg administered as an intravenous injection.

In some embodiments, the daratumumab-containing drug product label includes information that the recommended dosing schedule of daratumumab in combination with bortezomib, thalidomide and dexamethasone is once a week on weeks 1 to 8 and once in two weeks on weeks 9-24 during the induction phase and once every two weeks on weeks 1 to 8 during the consolidation phase.

In some embodiments, the daratumumab-containing drug product label includes information that the recommended dosing schedule of bortezomib is 1.3 mg/m² bortezomib on days 1, 4, 8 and 11 in the four 28-day induction cycles and on days 1, 4, 8 and 11 in the two 28-day consolidation cycles.

In some embodiments, the daratumumab-containing drug product label includes information that the recommended dosing schedule of thalidomide is 100 mg daily.

In some embodiments, the daratumumab-containing drug product label includes information that the recommended dosing schedule of dexamethasone is about 40 mg on days 1, 2, 8, 9, 15, 16 in the first and the second 28-day induction cycle, about 40 mg on days 1-2 and about 20 mg on days 8, 9, 15 and 16 in the third and the fourth 28-day induction cycle, and about 20 mg on days 1, 2, 8, 9, 15, 16 in the first and the second 28-day consolidation cycle.

In some embodiments, daratumumab, bortezomib, thalidomide and dexamethasone are administered according to the recommended dosing schedules.

In some embodiments, the daratumumab-containing drug product label includes data from an open-label, randomized active-controlled phase 3 study that compared treatment with daratumumab, bortezomib, thalidomide and dexamethasone (DVTd) to treatment with bortezomib, thalidomide and dexamethasone (VTd) in subjects with newly diagnosed multiple myeloma who are eligible for ASCT.

In some embodiments, the open-label, randomized active-controlled phase 3 study is known as CASSIOPEIA, listed at ClinicalTrials_gov database as study NCT02541383.

In some embodiments, the daratumumab-containing drug product label includes data that treatment with DVTd resulted in about 53% reduction in the risk of multiple myeloma progression or death when compared to treatment with VTd.

In some embodiments, the daratumumab-containing drug product label includes data that treatment with DVTd resulted in about 28.9% of subjects achieving the sCR or better, about 38.9% of subjects achieving the CR or better, and about 33.7% of subjects achieving a negative status for MRD, or any combination thereof.

In some embodiments, the daratumumab-containing drug product label includes a Kaplan-Meier curve of progression-free survival (PFS) comparing subjects having newly diagnosed multiple myeloma treated with DVTd to subjects having newly diagnosed multiple myeloma treated with VTd.

In some embodiments, the daratumumab-containing drug product label includes data from a phase 3 active-controlled study that compared treatment with daratumumab, bortezomib, melphalan and prednisone (D-VMP) to treatment with bortezomib, melphalan and prednisone (VMP) in subjects with newly diagnosed multiple myeloma.

In some embodiments, the phase 3 active-controlled study is known as ALCYONE, listed at ClinicalTrials_gov database as study NCT02195479.

In some embodiments, the daratumumab-containing drug product label includes data from a phase 3 active-controlled study that compared treatment with daratumumab, bortezomib, thalidomide and dexamethasone (DRd) to treatment with lenalidomide and dexamethasone (Rd) in relapsed, refractory or relapsed and refractory multiple myeloma.

In some embodiments, the phase 3 active-controlled study is known as POLLUX, listed at ClinicalTrials_gov database as study NCT02076009.

In some embodiments, the daratumumab-containing drug product label includes data from a phase 3 active-controlled study that compared treatment with daratumumab, bortezomib and dexamethasone (DVd) to treatment with bortezomid and dexamethasone (Vd) in relapsed, refractory or relapsed and refractory multiple myeloma.

In some embodiments, the phase 3 active-controlled study is known as CASTOR, listed at ClinicalTrials_gov database as study NCT02136134.

In some embodiments, the daratumumab-containing drug product label includes drug product interaction data informing that clinical pharmacokinetic assessments of daratumumab in combination with lenalidomide, pomalidomide bortezomib and dexamethasone indicated no clinically relevant drug-drug interactions between daratumumab and lenalidomide, pomalidomide bortezomib and dexamethasone.

In some embodiments, the daratumumab-containing drug product label includes information that side effects of daratumumab includes feeling weakness, decreased appetite, bronchitis and lung infection.

In some embodiments, the daratumumab-containing drug product label includes information about approved indications, dosage and administrations, adverse reactions, drug interactions, use in specific populations, clinical pharmacology, nonclinical toxicology, clinical studies and storage and handling of daratumumab, or any combination thereof.

In some embodiments, daratumumab is DARZALEX® brand of daratumumab.

In some embodiments, daratumumab is a biosimilar of DARZALEX® brand of daratumumab.

In some embodiments, daratumumab comprises a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6.

In some embodiments, daratumumab comprises a heavy chain variable region (VH) of SEQ ID NO: 7 and a light chain variable region (VL) of SEQ ID NO: 8.

In some embodiments, daratumumab is an immunoglobulin IgG1 kappa (IgG1κ).

An exemplary IgG1 constant domain sequence comprises an amino acid sequence of SEQ ID NO: 11. Some variation exists within the IgG1 constant domain (e.g. well-known allotypes), with variation at positions 214, 356, 358, 422, 431, 435 or 436 (residue numbering according to the EU numbering) (see e.g., IMGT Web resources; IMGT Repertoire (IG and TR); Proteins and alleles; allotypes). The antibody that specifically binds CD38 may be of any IgG1 allotype, such as Glm17, Glm3, Glm1, Glm2, Glm27 or Glm28.

In some embodiments, daratumumab comprises a heavy chain (HC) of SEQ ID NO: 9 and a light chain (LC) of SEQ ID NO: 10.

In some embodiments, daratumumab is produced in a mammalian cell line.

In some embodiments, the mammalian cell line is a Chinese hamster ovary (CHO) cell line.

In some embodiments, the molecular weight of daratumumab is about 148 kDa.

In some embodiments, dexamethasone can be substituted for a dexamethasone equivalent, wherein the dexamethasone equivalent is methylprednisolone, prednisolone, prednisone or betamethasone, or any combination thereof.

The disclosure also provides a method of treating a subject with newly diagnosed multiple myeloma, comprising:

providing a healthcare professional (HCP) daratumumab;
providing the HCP information that a combination therapy comprising daratumumab, bortezomib, thalidomide and dexamethasone is safe and effective in treating the subject with newly diagnosed multiple myeloma; wherein performing the steps a) and b) results in the subject with newly diagnosed multiple myeloma to receive a safe and effective combination therapy comprising daratumumab, bortezomib, thalidomide and dexamethasone by the HCP or by self-administration as instructed by the HCP, thereby treating the subject having the newly diagnosed multiple myeloma.

The disclosure also provides a method of providing daratumumab to a HCP for the HCP to treat a subject with newly diagnosed multiple myeloma with a safe and effective combination therapy comprising daratumumab, bortezomib, thalidomide and dexamethasone, comprising:

manufacturing daratumumab;
providing the HCP information that a combination therapy comprising daratumumab, bortezomib, thalidomide and dexamethasone is safe and effective in treating the subject with newly diagnosed multiple myeloma; and
shipping daratumumab to the HCP or to an authorized distributor of daratumumab for the HCP to purchase daratumumab to treat the subject with newly diagnosed multiple myeloma.

The disclosure also provides method of providing a treatment option for a HCP to treat a subject with newly diagnosed multiple myeloma with a safe and effective combination therapy comprising daratumumab, bortezomib, thalidomide and dexamethasone, comprising:

manufacturing daratumumab;
providing the HCP information that a combination therapy comprising daratumumab, bortezomib, thalidomide and dexamethasone is safe and effective in treating the subject with newly diagnosed multiple myeloma; and
shipping daratumumab to the HCP or to an authorized distributor of daratumumab for the HCP to purchase daratumumab.

In some embodiments, the subject is eligible for autologous stem cell transplant (ASCT). In eligible subjects, ASCT is provided in conjunction with high dose chemotherapy (HDC) as described herein.

In some embodiments, the safe and effective combination therapy comprising daratumumab, bortezomib, thalidomide and dexamethasone is demonstrated to increase a likelihood of achieving a stringent complete response (sCR) or better in subjects with newly diagnosed multiple myeloma.

In some embodiments, the likelihood of achieving the sCR or better is about 28% or higher.

In some embodiments, the safe and effective combination therapy comprising daratumumab, bortezomib, thalidomide and dexamethasone is demonstrated to increase a likelihood of achieving a complete response (CR) or better in subjects with newly diagnosed multiple myeloma.

In some embodiments, the likelihood of achieving the CR or better is about 38% or higher.

In some embodiments, the safe and effective combination therapy comprising daratumumab, bortezomib, thalidomide and dexamethasone is demonstrated to increase a likelihood of achieving a negative status for minimal residual disease (MRD) in subjects with newly diagnosed multiple myeloma.

In some embodiments, the likelihood of achieving the negative status for MRD is about 33% or higher.

In some embodiments, the safe and effective combination therapy comprising daratumumab, bortezomib, thalidomide and dexamethasone is demonstrated to reduce a risk of progression of multiple myeloma or death in subjects with newly diagnosed multiple myeloma.

In some embodiments, the risk of progression of multiple myeloma or death is reduced by about 53%.

In some embodiments, the safe and effective combination therapy comprises about 16 mg/kg daratumumab, about 1.3 mg/m² bortezomib, about 100 mg thalidomide and between about 20 mg and about 40 mg dexamethasone.

In some embodiments, the safe and effective combination therapy comprises an induction phase, a high dose chemotherapy (HDC) and autologous stem cell transplant (ASCT), and a consolidation phase.

In some embodiments, the induction phase comprises four 28-day induction cycles comprising about 16 mg/kg daratumumab administered once a week on weeks 1 to 8 and once in two weeks on weeks 9-16;

about 1.3 mg/m² bortezomib administered twice a week on week 1 and week 2 in the four 28-day induction cycles;
about 100 mg thalidomide daily; and
about 40 mg dexamethasone administered twice a week on week 1, week 2 and week 3 in the first and the second 28-day induction cycle, about 40 mg twice a week on week 1 and about 20 mg twice a week on week 2 and 3 in the third and the fourth 28-day induction cycle.

In some embodiments, the induction phase comprises four 28-day induction cycles comprising

about 16 mg/kg daratumumab administered once a week on weeks 1 to 8 and once in two weeks on weeks 9-16;
about 1.3 mg/m² bortezomib administered on days 1, 4, 8 and 11 in the four 28-day induction cycle;
about 100 mg thalidomide daily; and
about 40 mg dexamethasone administered on days 1, 2, 8, 9, 15, 16 in the first and the second 28-day induction cycle, about 40 mg on days 1 and 2 and about 20 mg on days 8, 9, 15 and 16 in the third and the fourth 28-day induction cycle.

In some embodiments, the induction phase is followed by the HDC and ASCT.

In some embodiments, the HDC comprises melphalan.

In some embodiments, melphalan is administered at a dose of about 200 mg/m², optionally over a period of 24 to 48 hours.

In some embodiments, the HDC and ASCT is followed by the consolidation phase.

In some embodiments, the consolidation phase comprises two 28-day consolidation cycles comprising

about 16 mg/kg daratumumab administered once in two weeks on weeks 1 to 8;
about 1.3 mg/m² bortezomib administered twice a week on week 1 and week 2 in each two 28-day consolidation cycle;
about 100 mg thalidomide daily; and
about 20 mg dexamethasone administered twice a week on week 1, week 2 and week 3 in each two 28-day consolidation cycle.

In some embodiments, the consolidation phase comprises two 28-day consolidation cycles of

about 16 mg/kg daratumumab on days 1 and 15 in each two 28-day consolidation cycle;
about 1.3 mg/m² bortezomib on days 1, 4, 8 and 11 in each two 28-day consolidation cycles;
about 100 mg thalidomide daily; and
about 20 mg dexamethasone on days 1, 2, 8, 9, 15 and 16 in each two 28-day consolidation cycles.

In some embodiments, the safe and effective combination therapy comprises administering dexamethasone as pre-medication on daratumumab administration days.

In some embodiments, the safe and effective combination therapy comprises administering daratumumab intravenously, bortezomib subcutaneously or intravenously, thalidomide orally and dexamethasone intravenously or orally.

In some embodiments, thalidomide, dexamethasone or both thalidomide and dexamethasone are self-administered.

In some embodiments, daratumumab is shipped or provided by a manufacturer of daratumumab in a single-dose vial comprising 100 mg daratumumab in 5 mL of solution or in a single-dose vial comprising 400 mg daratumumab in 20 mL of solution.

In some embodiments, each single-dose vial comprising 100 mg daratumumab in 5 mL of solution and each single-dose vial comprising 400 mg daratumumab in 20 mL of solution further comprises glacial acetic acid, mannitol, polysorbate 20, sodium acetate trihydrate and sodium chloride.

In some embodiments, each single-dose vial comprising 100 mg daratumumab in 5 mL of solution contains 0.9 mg glacial acetic acid, 127.5 mg mannitol, 2 mg polysorbate 20, 14.8 mg sodium acetate trihydrate, 17.5 mg sodium chloride and water for injection, and each single-dose vial comprising 400 mg daratumumab in 20 mL of solution contains 400 mg daratumumab, 3.7 mg glacial acetic acid, 510 mg mannitol, 8 mg polysorbate 20, 59.3 mg sodium acetate trihydrate, 70.1 mg sodium chloride and water for injection.

In some embodiments, daratumumab is diluted into 0.9% sodium chloride prior to administration.

In some embodiments, information that a combination therapy comprising daratumumab, bortezomib, thalidomide and dexamethasone is safe and effective is provided in a daratumumab-containing drug product label.

In some embodiments, the daratumumab-containing drug product label includes information that a recommended dose of daratumumab is 16 mg/kg administered as an intravenous injection.

In some embodiments, the daratumumab-containing drug product label includes information that the recommended dosing schedule of daratumumab in combination with bortezomib, thalidomide and dexamethasone is once a week on weeks 1 to 8 and once in two weeks on weeks 9-24 during the induction phase and once every two weeks on weeks 1 to 8 during the consolidation phase.

In some embodiments, the daratumumab-containing drug product label includes information that the recommended dosing schedule of bortezomib is 1.3 mg/m² bortezomib on days 1, 4, 8 and 11 in the four 28-day induction cycles and on days 1, 4, 8 and 11 in the two 28-day consolidation cycles.

In some embodiments, the daratumumab-containing drug product label includes information that the recommended dosing schedule of thalidomide is 100 mg daily.

In some embodiments, the daratumumab-containing drug product label includes information that the recommended dosing schedule of dexamethasone is about 40 mg on days 1, 2, 8, 9, 15, 16 in the first and the second 28-day induction cycle, about 40 mg on days 1-2 and about 20 mg on days 8, 9, 15 and 16 in the third and the fourth 28-day induction cycle, and about 20 mg on days 1, 2, 8, 9, 15, 16 in the first and the second 28-day consolidation cycle.

In some embodiments, daratumumab, bortezomib, thalidomide and dexamethasone are administered according to the recommended dosing schedules.

In some embodiments, the daratumumab-containing drug product label includes data from an open-label, randomized active-controlled phase 3 study that compared treatment with daratumumab, bortezomib, thalidomide and dexamethasone (DVTd) to treatment with bortezomib, thalidomide and dexamethasone (VTd) in subjects with newly diagnosed multiple myeloma who are eligible for ASCT.

In some embodiments, the open-label, randomized active-controlled phase 3 study is known as CASSIOPEIA, listed at ClinicalTrials_gov database as study NCT02541383.

In some embodiments, the daratumumab-containing drug product label includes data that treatment with DVTd resulted in about 53% reduction in the risk of multiple myeloma progression or death when compared to treatment with VTd.

In some embodiments, the daratumumab-containing drug product label includes data that treatment with DVTd resulted in about 28.9% of subjects achieving the sCR or better, about 38.9% of subjects achieving the CR or better, and about 33.7% of subjects achieving a negative status for MRD, or any combination thereof.

In some embodiments, the daratumumab-containing drug product label includes a Kaplan-Meier curve of progression-free survival (PFS) comparing subjects having newly diagnosed multiple myeloma treated with DVTd to subjects having newly diagnosed multiple myeloma treated with VTd.

In some embodiments, the daratumumab-containing drug product label includes data from a phase 3 active-controlled study that compared treatment with daratumumab, bortezomib, melphalan and prednisone (D-VMP) to treatment with bortezomib, melphalan and prednisone (VMP) in subjects with newly diagnosed multiple myeloma.

In some embodiments, the phase 3 active-controlled study is known as ALCYONE, listed at ClinicalTrials_gov database as study NCT02195479.

In some embodiments, the daratumumab-containing drug product label includes data from a phase 3 active-controlled study that compared treatment with daratumumab, bortezomib, thalidomide and dexamethasone (DRd) to treatment with lenalidomide and dexamethasone (Rd) in relapsed, refractory or relapsed and refractory multiple myeloma.

In some embodiments, the phase 3 active-controlled study is known as POLLUX, listed at ClinicalTrials_gov database as study NCT02076009.

In some embodiments, the daratumumab-containing drug product label includes data from a phase 3 active-controlled study that compared treatment with daratumumab, bortezomib and dexamethasone (DVd) to treatment with bortezomid and dexamethasone (Vd) in relapsed, refractory or relapsed and refractory multiple myeloma.

In some embodiments, the phase 3 active-controlled study is known as CASTOR, listed at ClinicalTrials_gov database as study NCT02136134.

In some embodiments, the daratumumab-containing drug product label includes drug product interaction data informing that clinical pharmacokinetic assessments of daratumumab in combination with lenalidomide, pomalidomide, bortezomib and dexamethasone indicated no clinically relevant drug-drug interactions between daratumumab and lenalidomide, pomalidomide, bortezomib and dexamethasone.

In some embodiments, the daratumumab-containing drug product label includes information that side effects of daratumumab includes feeling weakness, decreased appetite, bronchitis and lung infection.

In some embodiments, the daratumumab-containing drug product label includes information about approved indications, dosage and administrations, adverse reactions, drug interactions, use in specific populations, clinical pharmacology, nonclinical toxicology, clinical studies and storage and handling of daratumumab, or any combination thereof.

In some embodiments, daratumumab is DARZALEX® brand of daratumumab.

In some embodiments, daratumumab is a biosimilar of DARZALEX® brand of daratumumab.

In some embodiments, daratumumab comprises a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6.

In some embodiments, daratumumab comprises a heavy chain variable region (VH) of SEQ ID NO: 7 and a light chain variable region (VL) of SEQ ID NO: 8.

In some embodiments, daratumumab is an immunoglobulin IgG1 kappa (IgG1κ).

An exemplary IgG1 constant domain sequence comprises an amino acid sequence of SEQ ID NO: 11. Some variation exists within the IgG1 constant domain (e.g. well-known allotypes), with variation at positions 214, 356, 358, 422, 431, 435 or 436 (residue numbering according to the EU numbering) (see e.g., IMGT Web resources; IMGT Repertoire (IG and TR); Proteins and alleles; allotypes). The antibody that specifically binds CD38 may be of any IgG1 allotype, such as Glm17, Glm3, Glm1, Glm2, Glm27 or Glm28.

In some embodiments, daratumumab comprises a heavy chain (HC) of SEQ ID NO: 9 and a light chain (LC) of SEQ ID NO: 10.

In some embodiments, daratumumab is produced in a mammalian cell line.

In some embodiments, the mammalian cell line is a Chinese hamster ovary (CHO) cell line.

In some embodiments, the molecular weight of daratumumab is about 148 kDa.

In some embodiments, dexamethasone can be substituted for a dexamethasone equivalent, wherein the dexamethasone equivalent is methylprednisolone, prednisolone, prednisone or betamethasone, or any combination thereof.

### Safe and Effective Combination Therapies and Drug Products of the Disclosure

The disclosure provides a safe and effective combination therapy comprising daratumumab, bortezomib, thalidomide and dexamethasone for providing a safe and effective treatment of a subject with newly diagnosed multiple myeloma.

In some embodiments, the safe and effective combination therapy of the disclosure comprises about 16 mg/kg daratumumab, about 1.3 mg/m² bortezomib, about 100 mg thalidomide and between about 20 mg and about 40 mg dexamethasone.

In some embodiments, the subject with newly diagnosed multiple myeloma is eligible for autologous stem cell transplant (ASCT).

In some embodiments, the safe and effective treatment of the subject with newly diagnosed multiple myeloma comprises an induction phase, a high dose chemotherapy (HDC) and autologous stem cell transplant (ASCT), and a consolidation phase.

In some embodiments, the induction phase comprises four 28-day induction cycles comprising

about 16 mg/kg daratumumab administered once a week on weeks 1 to 8 and once in two weeks on weeks 9-16;
about 1.3 mg/m² bortezomib administered twice a week on week 1 and week 2 in the four 28-day induction cycles;
about 100 mg thalidomide daily; and
about 40 mg dexamethasone administered twice a week on week 1, week 2 and week 3 in the first and the second 28-day induction cycle, about 40 mg twice a week on week 1 and about 20 mg twice a week on week 2 and 3 in the third and the fourth 28-day induction cycle.

In some embodiments, the induction phase comprises four 28-day induction cycles comprising

about 16 mg/kg daratumumab administered once a week on weeks 1 to 8 and once in two weeks on weeks 9-16;
about 1.3 mg/m² bortezomib administered on days 1, 4, 8 and 11 in the four 28-day induction cycle;
about 100 mg thalidomide daily; and
about 40 mg dexamethasone administered on days 1, 2, 8, 9, 15, 16 in the first and the second 28-day induction cycle, about 40 mg on days 1 and 2 and about 20 mg on days 8, 9, 15 and 16 in the third and the fourth 28-day induction cycle.

In some embodiments, the induction phase is followed by the HDC and ASCT.

In some embodiments, the HDC comprises melphalan.

In some embodiments, melphalan is administered at a dose of about 200 mg/m², optionally over a period of 24 to 48 hours.

In some embodiments, the HDC and ASCT is followed by the consolidation phase.

In some embodiments, the consolidation phase comprises two 28-day consolidation cycles comprising

about 16 mg/kg daratumumab administered once in two weeks on weeks 1 to 8;
about 1.3 mg/m² bortezomib administered twice a week on week 1 and week 2 in each two 28-day consolidation cycle;
about 100 mg thalidomide daily; and
about 20 mg dexamethasone administered twice a week on week 1, week 2 and week 3 in each two 28-day consolidation cycle.

In some embodiments, the consolidation phase comprises two 28-day consolidation cycles of

about 16 mg/kg daratumumab on days 1 and 15 in each two 28-day consolidation cycle;
about 1.3 mg/m² bortezomib on days 1, 4, 8 and 11 in each two 28-day consolidation cycles;
about 100 mg thalidomide daily; and
about 20 mg dexamethasone on days 1, 2, 8, 9, 15 and 16 in each two 28-day consolidation cycles.

In some embodiments, dexamethasone is administered as pre-medication on daratumumab administration days.

In some embodiments, daratumumab is administered intravenously, bortezomib is administered subcutaneously or intravenously, thalidomide is administered orally and dexamethasone is administered intravenously or orally.

In some embodiments, thalidomide, dexamethasone or both thalidomide and dexamethasone are self-administered.

In some embodiments the safe and effective combination therapy of the disclosure is demonstrated to increase a likelihood of achieving a stringent complete response (sCR) or better in subjects with newly diagnosed multiple myeloma.

In some embodiments, the likelihood of achieving the sCR or better is about 28% or more.

In some embodiments the safe and effective combination therapy of the disclosure is demonstrated to increase a likelihood of achieving a complete response (CR) or better in subjects with newly diagnosed multiple myeloma.

In some embodiments, the likelihood of achieving the CR or better is about 38% or more.

In some embodiments the safe and effective combination therapy of the disclosure is demonstrated to increase a likelihood of achieving a negative status for minimal residual disease (MRD) in subjects with newly diagnosed multiple myeloma.

In some embodiments, the likelihood of achieving the negative status for MRD is about 33% or more.

In some embodiments the safe and effective combination therapy of the disclosure is demonstrated to reduce a risk of progression of multiple myeloma or death in subjects with newly diagnosed multiple myeloma.

In some embodiments, the risk of progression of multiple myeloma or death is reduced by about 53%.

In some embodiments, the safe and effective combination therapy of the disclosure is promoted by a manufacturer of daratumumab for treatment of newly diagnosed multiple myeloma on a daratumumab-containing drug product label.

In some embodiments, the daratumumab-containing drug product label includes data from an open-label, randomized active-controlled phase 3 study that compared treatment with daratumumab, bortezomib, thalidomide and dexamethasone (DVTd) to treatment with bortezomib, thalidomide and dexamethasone (VTd) in subjects with newly diagnosed multiple myeloma who are eligible for ASCT.

In some embodiments, the open-label, randomized active-controlled phase 3 study is known as CASSIOPEIA, listed at ClinicalTrials_gov database as study NCT02541383.

In some embodiments, the daratumumab-containing drug product label includes data that treatment with DVTd resulted in about 53% reduction in the risk of multiple myeloma progression or death when compared to treatment with VTd.

In some embodiments, the daratumumab-containing drug product label includes data that treatment with DVTd resulted in about 28.9% of subjects achieving the sCR or better, about 38.9% of subjects achieving the CR or better, and about 33.7% of subjects achieving a negative status for MRD, or any combination thereof.

In some embodiments, the daratumumab-containing drug product label includes a Kaplan-Meier curve of progression-free survival (PFS) comparing subjects having newly diagnosed multiple myeloma treated with DVTd to subjects having newly diagnosed multiple myeloma treated with VTd.

In some embodiments, the daratumumab-containing drug product label includes data from a phase 3 active-controlled study that compared treatment with daratumumab, bortezomib, melphalan and prednisone (D-VMP) to treatment with bortezomib, melphalan and prednisone (VMP) in subjects with newly diagnosed multiple myeloma.

In some embodiments, the phase 3 active-controlled study is known as ALCYONE, listed at ClinicalTrials_gov database as study NCT02195479.

In some embodiments, the daratumumab-containing drug product label includes data from a phase 3 active-controlled study that compared treatment with daratumumab, bortezomib, thalidomide and dexamethasone (DRd) to treatment with lenalidomide and dexamethasone (Rd) in relapsed, refractory or relapsed and refractory multiple myeloma.

In some embodiments, the phase 3 active-controlled study is known as POLLUX, listed at ClinicalTrials_gov database as study NCT02076009.

In some embodiments, the daratumumab-containing drug product label includes data from a phase 3 active-controlled study that compared treatment with daratumumab, bortezomib and dexamethasone (DVd) to treatment with bortezomid and dexamethasone (Vd) in relapsed, refractory or relapsed and refractory multiple myeloma.

In some embodiments, the phase 3 active-controlled study is known as CASTOR, listed at ClinicalTrials_gov database as study NCT02136134.

In some embodiments, the daratumumab-containing drug product label includes drug product interaction data informing that clinical pharmacokinetic assessments of daratumumab in combination with lenalidomide, pomalidomide, bortezomib and dexamethasone indicated no clinically relevant drug-drug interactions between daratumumab and lenalidomide, pomalidomide, bortezomib and dexamethasone.

In some embodiments, the daratumumab-containing drug product label includes information that side effects of daratumumab includes weakness, decreased appetite, bronchitis and lung infection.

In some embodiments, the daratumumab-containing drug product label includes information about approved indications, dosage and administrations, adverse reactions, drug interactions, use in specific populations, clinical pharmacology, nonclinical toxicology, clinical studies and storage and handling of daratumumab, or any combination thereof.

In some embodiments, daratumumab is DARZALEX® brand of daratumumab.

In some embodiments, daratumumab is a biosimilar of DARZALEX® brand of daratumumab.

In some embodiments, daratumumab comprises a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6.

In some embodiments, daratumumab comprises a heavy chain variable region (VH) of SEQ ID NO: 7 and a light chain variable region (VL) of SEQ ID NO: 8.

In some embodiments, daratumumab is an immunoglobulin IgG1 kappa (IgG1κ).

An exemplary IgG1 constant domain sequence comprises an amino acid sequence of SEQ ID NO: 11. Some variation exists within the IgG1 constant domain (e.g. well-known allotypes), with variation at positions 214, 356, 358, 422, 431, 435 or 436 (residue numbering according to the EU numbering) (see e.g., IMGT Web resources; IMGT Repertoire (IG and TR); Proteins and alleles; allotypes). The antibody that specifically binds CD38 may be of any IgG1 allotype, such as Glm17, Glm3, Glm1, Glm2, Glm27 or Glm28.

In some embodiments, daratumumab comprises a heavy chain (HC) of SEQ ID NO: 9 and a light chain (LC) of SEQ ID NO: 10.

In some embodiments, daratumumab is produced in a mammalian cell line.

In some embodiments, the mammalian cell line is a Chinese hamster ovary (CHO) cell line.

In some embodiments, the molecular weight of daratumumab is about 148 kDa.

In some embodiments, dexamethasone can be substituted for a dexamethasone equivalent, wherein the dexamethasone equivalent is methylprednisolone, prednisolone, prednisone or betamethasone, or any combination thereof.

The disclosure also provides a drug product comprising daratumumab that is provided in a package comprising one or more single-dose vials comprising daratumumab and a drug product label that includes information that a combination therapy comprising daratumumab, bortezomib, thalidomide and dexamethasone is safe and effective for treatment of a subject with newly diagnosed multiple myeloma.

In some embodiments, the one or more single-dose vials comprises 100 mg daratumumab in 5 mL of solution or 400 mg daratumumab in 20 mL of solution.

In some embodiments, the one or more single-dose vials comprising 100 mg daratumumab in 5 mL of solution and the one or more single-dose vials comprising 400 mg daratumumab in 20 mL of solution further comprises glacial acetic acid, mannitol, polysorbate 20, sodium acetate trihydrate and sodium chloride.

In some embodiments, the one or more single-dose vials comprising 100 mg daratumumab in 5 mL of solution contains 0.9 mg glacial acetic acid, 127.5 mg mannitol, 2 mg polysorbate 20, 14.8 mg sodium acetate trihydrate, 17.5 mg sodium chloride and water for injection, and the one or more single-dose vials comprising 400 mg daratumumab in 20 mL of solution contains 400 mg daratumumab, 3.7 mg glacial acetic acid, 510 mg mannitol, 8 mg polysorbate 20, 59.3 mg sodium acetate trihydrate, 70.1 mg sodium chloride and water for injection.

In some embodiments, the drug product label includes information that a recommended dosing schedule of daratumumab in combination with bortezomib, thalidomide and dexamethasone is once a week on weeks 1 to 8 and once in two weeks on weeks 9-24 during an induction phase and once every two weeks on weeks 1 to 8 during a consolidation phase.

In some embodiments, the induction phase comprises four 28-day induction cycles comprising about 16 mg/kg daratumumab administered once a week on weeks 1 to 8 and once in two weeks on weeks 9-16;

about 1.3 mg/m² bortezomib administered on days 1, 4, 8 and 11 in the four 28-day induction cycle;
about 100 mg thalidomide daily; and
about 40 mg dexamethasone administered on days 1, 2, 8, 9, 15, 16 in the first and the second 28-day induction cycle, about 40 mg on days 1 and 2 and about 20 mg on days 8, 9, 15 and 16 in the third and the fourth 28-day induction cycle.

In some embodiments, the induction phase is followed by the HDC and ASCT.

In some embodiments, the HDC comprises melphalan.

In some embodiments, melphalan is administered at a dose of about 200 mg/m², optionally over a period of 24 to 48 hours.

In some embodiments, the consolidation phase comprises two 28-day consolidation cycles of

about 16 mg/kg daratumumab on days 1 and 15 in each two 28-day consolidation cycle;
about 1.3 mg/m² bortezomib on days 1, 4, 8 and 11 in each two 28-day consolidation cycles;
about 100 mg thalidomide daily; and
about 20 mg dexamethasone on days 1, 2, 8, 9, 15 and 16 in each two 28-day consolidation cycles.

In some embodiments, the drug product label includes data from an open-label, randomized active-controlled phase 3 study that compared treatment with daratumumab, bortezomib, thalidomide and dexamethasone (DVTd) to treatment with bortezomib, thalidomide and dexamethasone (VTd) in subjects with newly diagnosed multiple myeloma who are eligible for ASCT.

In some embodiments, the open-label, randomized active-controlled phase 3 study is known as CASSIOPEIA, listed at ClinicalTrials_gov database as study NCT02541383.

In some embodiments, the drug product label includes data that treatment with DVTd resulted in about 53% reduction in the risk of multiple myeloma progression or death when compared to treatment with VTd.

In some embodiments, the drug product label includes data that treatment with DVTd resulted in about 28.9% of subjects achieving the sCR or better, about 38.9% of subjects achieving the CR or better, and about 33.7% of subjects achieving a negative status for MRD, or any combination thereof.

In some embodiments, the drug product label includes a Kaplan-Meier curve of progression-free survival (PFS) comparing subjects having newly diagnosed multiple myeloma treated with DVTd to subjects having newly diagnosed multiple myeloma treated with VTd.

In some embodiments, the drug product label includes data from a phase 3 active-controlled study that compared treatment with daratumumab, bortezomib, melphalan and prednisone (D-VMP) to treatment with bortezomib, melphalan and prednisone (VMP).

In some embodiments, the phase 3 active-controlled study is known as ALCYONE, listed at ClinicalTrials_gov database as study NCT02195479.

In some embodiments, the drug product label includes data from a phase 3 active-controlled study that compared treatment with daratumumab in combination with lenalidomide and dexamethasone (DRd) to treatment with lenalidomide and dexamethasone (Rd) in relapsed, refractory or relapsed and refractory multiple myeloma.

In some embodiments, the phase 3 active-controlled study is known as POLLUX, listed at ClinicalTrials_gov database as study NCT02076009.

In some embodiments, the drug product label includes data from a phase 3 active-controlled study that compared treatment with daratumumab in combination with bortezomib and dexamethasone (DVd) to treatment with bortezomid and dexamethasone (Vd) in relapsed, refractory or relapsed and refractory multiple myeloma.

In some embodiments, the phase 3 active-controlled study is known as CASTOR, listed at ClinicalTrials_gov database as study NCT02136134.

In some embodiments, the drug product label includes drug interaction data informing that clinical pharmacokinetic assessments of daratumumab in combination with lenalidomide, pomalidomide, bortezomib and dexamethasone indicated no clinically relevant drug-drug interactions between daratumumab and lenalidomide, pomalidomide, bortezomib and dexamethasone.

In some embodiments, the drug product label includes information that side effects of daratumumab includes feeling weakness, decreased appetite, bronchitis and lung infection.

In some embodiments, the drug product label includes information about approved indications, dosage and administrations, adverse reactions, drug interactions, use in specific populations, clinical pharmacology, nonclinical toxicology, clinical studies and storage and handling of daratumumab, or any combination thereof.

In some embodiments, daratumumab is DARZALEX® brand of daratumumab.

In some embodiments, daratumumab is a biosimilar of DARZALEX® brand of daratumumab.

In some embodiments, daratumumab comprises a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6.

In some embodiments, daratumumab comprises a heavy chain variable region (VH) of SEQ ID NO: 7 and a light chain variable region (VL) of SEQ ID NO: 8.

In some embodiments, daratumumab is an immunoglobulin IgG1 kappa (IgG1κ).

An exemplary IgG1 constant domain sequence comprises an amino acid sequence of SEQ ID NO: 11. Some variation exists within the IgG1 constant domain (e.g. well-known allotypes), with variation at positions 214, 356, 358, 422, 431, 435 or 436 (residue numbering according to the EU numbering) (see e.g., IMGT Web resources; IMGT Repertoire (IG and TR); Proteins and alleles; allotypes). The antibody that specifically binds CD38 may be of any IgG1 allotype, such as Glm17, Glm3, Glm1, Glm2, Glm27 or Glm28.

In some embodiments, daratumumab comprises a heavy chain (HC) of SEQ ID NO: 9 and a light chain (LC) of SEQ ID NO: 10.

In some embodiments, daratumumab is produced in a mammalian cell line.

In some embodiments, the mammalian cell line is a Chinese hamster ovary (CHO) cell line.

In some embodiments, the molecular weight of daratumumab is about 148 kDa.

The disclosure also provides a method of selling a drug product comprising daratumumab, comprising:

manufacturing daratumumab;
promoting that a combination therapy comprising daratumumab, bortezomib, thalidomide and dexamethasone is safe and effective for treatment of a subject with newly diagnosed multiple myeloma, wherein performing the steps a) and b) results in a health care professional (HCP) to purchase the drug product; thereby selling the drug product.

In some embodiments, promoting comprises including data from an open-label, randomized active-controlled phase 3 study that compared treatment with daratumumab, bortezomib, thalidomide and dexamethasone (DVTd) to treatment with bortezomib, thalidomide and dexamethasone (VTd) in subjects with newly diagnosed multiple myeloma who are eligible for ASCT on the drug product label.

In some embodiments, the open-label, randomized active-controlled phase 3 study is known as CASSIOPEIA, listed at ClinicalTrials_gov database as study NCT02541383.

In some embodiments, the drug product label further includes data that treatment with DVTd resulted in about 53% reduction in the risk of multiple myeloma progression or death when compared to treatment with VTd.

In some embodiments, the drug product label further includes a Kaplan-Meier curve of progression-free survival (PFS) comparing subjects having newly diagnosed multiple myeloma treated with DVTd to subjects having newly diagnosed multiple myeloma treated with VTd.

The disclosure also provides a method of selling a drug product comprising daratumumab, comprising

manufacturing daratumumab;
selling the drug product, wherein the drug product label includes an indication for treating a subject with newly diagnosed multiple myeloma with a combination of daratumumab, bortezomib, thalidomide and dexamethasone.

In some embodiments, daratumumab is DARZALEX® brand of daratumumab.

In some embodiments, daratumumab is a biosimilar of DARZALEX® brand of daratumumab.

In some embodiments, daratumumab comprises a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6.

In some embodiments, daratumumab comprises a heavy chain variable region (VH) of SEQ ID NO: 7 and a light chain variable region (VL) of SEQ ID NO: 8.

In some embodiments, daratumumab is an immunoglobulin IgG1 kappa (IgG1κ). The method of any one of claims 74-83, wherein daratumumab comprises a heavy chain (HC) of SEQ ID NO: 9 and a light chain (LC) of SEQ ID NO: 10.

In some embodiments, daratumumab is produced in a mammalian cell line.

In some embodiments, the mammalian cell line is a Chinese hamster ovary (CHO) cell line.

In some embodiments, the molecular weight of daratumumab is about 148 kDa.

### Methods of Producing Antibodies

Methods of producing antibodies at large scales are known. Antibodies may be produced for example in CHO cells cultured using known methods. The antibody may be isolated and/or purified from culture medium by removing solids by centrifugation or filtering as a first step in the purification process. The antibody may be further purified by standard methods including chromatography (e.g., ion exchange, affinity, size exclusion, and hydroxyapatite chromatography), gel filtration, centrifugation, or differential solubility, ethanol precipitation or by any other available technique for the purification of antibodies. Protease inhibitors such as phenyl methyl sulfonyl fluoride (PMSF), leupeptin, pepstatin or aprotinin can be added at any or all stages in order to reduce or eliminate degradation of the antibody during the purification process. One of ordinary skill in the art will appreciate that the exact purification technique will vary depending on the character of the polypeptide or protein to be purified, the character of the cells from which the polypeptide or protein is expressed, and the composition of the medium in which the cells were grown.

The purified antibody is formulated in a pharmaceutical composition comprising one or more excipients and packaged into a container such as a sealed bottle or vessel, such as a glass vial, with label affixed to the container or included in the package. Alternatively, the purified antibody may be lyophilized and provided as a lyophilized powder in the container.

While having described the invention in general terms, the embodiments of the invention will be further disclosed in the following examples that should not be construed as limiting the scope of the claims.

### Example 1: Phase 3 Study of DARZALEX® (Daratumumab) in Combination with Bortezomib, Thalidomide and Dexamethasone (D-VTD) in the First Line Treatment of Transplant Eligible Subjects with Newly Diagnosed Multiple Myeloma (CASSIOPEIA) (NCT02541383)

### Overview of Study Design

This is a randomized, open-label, active control, parallel group, multicenter, Phase 3 study in subjects with previously untreated multiple myeloma. The planned number of subjects to be treated in this study is as follows:

1080 subjects (540/arm) for first randomization (induction)

Approximately 800 subjects (400/arm) of the initial 1080 subjects will be randomized to maintenance. The actual accrual into the Maintenance Phase may be greater than 800 if a higher-than-expected proportion of subjects in the induction/consolidation stage achieve response and are randomized in the Maintenance Phase.

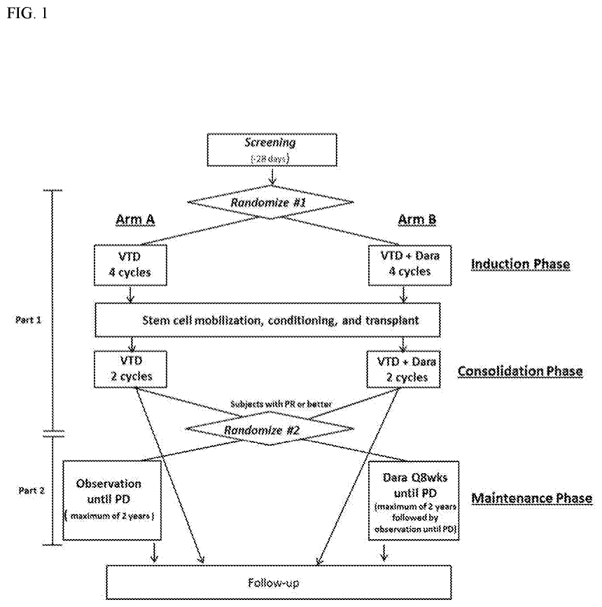

The study will consist of 3 phases. The Screening Phase will extend up to 28 days prior to Cycle 1, Day 1. The Treatment Phase will be conducted in 2 parts, as described below, and will extend from Cycle 1 Day 1 until treatment discontinuation due to progressive disease, unacceptable toxicity, ineligibility for second randomization, or 2 years of maintenance therapy/observation. The Follow-up (FU) Phase will extend from treatment discontinuation until death, loss to follow-up, withdrawal of consent, or study end, whichever occurs first. FIG. 1 shows the study design.

The 2 parts in the Treatment Phase are described below.

### Part 1: Induction/ASCT/Consolidation Phase (1:1 Randomization)

Arm A: VTD induction therapy (4 cycles), followed by ASCT, followed by 2 cycles of VTD consolidation
Arm B: VTD plus DARZALEX® (daratumumab) induction therapy (4 cycles), followed by ASCT, followed by 2 cycles of VTD plus DARZALEX® (daratumumab) consolidation

The consolidation phase of treatment will begin approximately 30 days after ASCT, when the subject has recovered sufficiently and engraftment is complete. Response will be evaluated at Day 100 post ASCT.

Part 2: Maintenance Phase (1:1 Re-Randomization of Subjects Achieving at Least a PR after Consolidation)

Subjects with at least a PR will be randomized after determination of response at approximately Day 100 after ASCT and will enter the Maintenance Phase upon completion of consolidation therapy. Subjects who have not achieved a response will enter the Follow-up Phase and will be followed until disease progression or death, even if they receive subsequent treatment.
Arm A: Observation only until documented disease progression (limited to 2 years maximum duration)
Arm B: DARZALEX® (daratumumab) monotherapy until documented disease progression (limited to 2 years maximum duration)

### Primary Objectives:

The primary objective in Part 1 is to determine if the addition of DARZALEX® (daratumumab) to VTD will increase the proportion of subjects achieving stringent complete response (sCR) post completion of consolidation therapy compared with VTD alone.

The primary objective in Part 2 is to determine if the use of DARZALEX® (daratumumab) as single agent in maintenance compared to observation only will increase progression-free survival (PFS) when used after autologous stem cell transplant and consolidation therapy.

### Secondary Objectives:

In Part 1, major secondary efficacy objectives are to determine if the addition of DARZALEX® (daratumumab) to VTD will improve:
Progression-free survival (PFS) from first randomization
Time to progression (TTP) from first randomization
Complete response (CR) rate by the end of ASCT/consolidation
Minimal residual disease (MRD) negative rate by the end of ASCT/consolidation
Post-induction stringent complete response (sCR) rate
Progression-free survival after next line of therapy (PFS2)
Post-induction overall response rate (ORR) and rate of very good partial response (VGPR) or better
Overall survival (OS)
Duration of CR and sCR

In Part 2, major secondary efficacy objectives are to determine if the addition of DARZALEX® (daratumumab) to VTD will improve the assessment during maintenance of:
Time to progression
CR rate
MRD negative rate
PFS2
Rate of improved response
Rate of MRD negative conversion
ORR
OS

### Other Secondary Objectives Throughout the Study are:

To evaluate quality of life and health economic/resource utilization
To assess immunogenicity of DARZALEX® (daratumumab)
To assess safety and tolerability of DARZALEX® (daratumumab) in combination with VTD

### Exploratory Objectives:

To evaluate impact of DARZALEX® (daratumumab) on response and resistance to treatment

In Arm B, DARZALEX® (daratumumab) (16 mg/kg) will be administered by intravenous (IV) infusion once every week for 8 weeks (VTD Induction Cycle 1-2), then once every 2 weeks for 8 weeks (VTD Induction Cycle 3-4) and following ASCT once every 2 weeks for 8 weeks (VTD consolidation Cycle 5-6). Following subsequent re randomization, subjects assigned to the maintenance Arm B will receive DARZALEX® (daratumumab) (16 mg/kg) once every 8 weeks until documented disease progression (limited to a maximum duration of 2 years).

Permuted block randomization will be implemented in this study. Subjects will be stratified at first randomization by site affiliation (IFM or HOVON), International Staging System stage I, II, or III (β-2 microglobulin and albumin) and by cytogenetics (standard risk or high risk as defined by presence of del17p or t(4;14), as centrally confirmed during screening).

Response will be assessed 100 days after ASCT and eligibility for the second randomization will be determined. Subjects will be stratified at the second randomization by type of induction treatment (VTD+/−DARZALEX® (daratumumab)) and by depth of response to induction/consolidation therapy (as determined by MRD status and post-consolidation response).

Assessment of tumor response and disease progression will be conducted in accordance with the International Myeloma Working Group (IMWG) response criteria. An assessment of MRD will be conducted using NGS and flow cytometry on bone marrow aspirates for all patients in induction/consolidation phases and for patients who achieve at least VGPR in maintenance phase.

Safety evaluations will include adverse event monitoring, physical examinations, electrocardiogram monitoring (ECGs), clinical laboratory parameters (hematology and chemistry), vital sign measurements, and Eastern Cooperative Oncology Group (ECOG) performance status.

Two primary analyses are planned. The first primary analysis, with a purpose to evaluate response by measuring the stringent complete response (sCR) rate, will be performed after all subjects have completed the Day 100 post-ASCT response evaluation or have been discontinued from study treatment by this timepoint. The second primary analysis, for PFS, will be performed when approximately 390 PFS events have been observed.

A final data cutoff will occur at the end of study, when approximately 350 subjects have died, or approximately 5 years after the last subject is randomized in the second randomization, whichever comes first.

### Subject Population

### Inclusion Criteria

Each potential subject must satisfy all of the following criteria to be enrolled in the study.

1. Subject must be between 18 and 65 years of age.
2. Subject must have documented multiple myeloma satisfying the CRAB or biomarkers of malignancy criteria and measurable disease as defined by:
Monoclonal plasma cells in the bone marrow ≥10% or presence of a biopsy proven plasmacytoma AND any one or more of the following myeloma defining events:
   Hypercalcemia: serum calcium >0.25 mmol/L (>1 mg/dL) higher than ULN or >2.75 mmol/L (>11 mg/dL)
   Renal insufficiency: creatinine clearance <40 mL/min or serum creatinine >177 μmol/L (>2 mg/dL)
   Anemia: hemoglobin >2 g/dL below the lower limit of normal or hemoglobin <10 g/dL
   Bone lesions: one or more osteolytic lesions on skeletal radiography, CT, or PET-CT
   Clonal bone marrow plasma cell percentage ≥60%
   Involved: uninvolved serum free light chain ratio ≥100
   >1 focal lesion on MRI studies
Measurable disease as defined by any of the following:
   IgG multiple myeloma: Serum monoclonal paraprotein (M-protein) level ≥1.0 g/dL or urine M-protein level ≥200 mg/24 hours; or
   IgA, IgE, IgD, or IgM multiple myeloma: serum M-protein level ≥0.5 g/dL or urine M-protein level ≥200 mg/24 hours; or
   IgD multiple myeloma: serum M-protein level <0.5 g/dL and Serum immunoglobulin free light chain ≥10 mg/dL and abnormal serum immunoglobulin kappa lambda free light chain ratio; or
   Light chain multiple myeloma without measurable disease in the serum or the urine: Serum immunoglobulin free light chain ≥10 mg/dL and abnormal serum immunoglobulin kappa lambda free light chain ratio
      3. Newly diagnosed subjects eligible for high dose therapy and autologous stem cell transplantation.
      4. Subject must have an ECOG performance status score of 0, 1, or 2
      5. Subject must have pretreatment clinical laboratory values meeting the following criteria during the Screening Phase (Lab tests should be repeated if done more than 15 days before C1D1):
a) hemoglobin ≥7.5 g/dL (≥5 mmol/L; prior red blood cell [RBC] transfusion or recombinant human erythropoietin use is permitted);
b) absolute neutrophil count (ANC) ≥1.0×10⁹/L (GCSF use is permitted);
c) AST ≤2.5× upper limit of normal (ULN);
d) ALT <2.5×ULN;
e) total bilirubin ≤1.5×ULN (except in subjects with congenital bilirubinemia, such as Gilbert syndrome, direct bilirubin ≤1.5×ULN);
f) calculated creatinine clearance ≥40 mL/min/1.73 m²;
g) corrected serum calcium ≤14 mg/dL (<3.5 mmol/L); or free ionized calcium ≤6.5 mg/dL (≤1.6 mmol/L)
h) platelet count ≥70×10⁹/L for subjects in whom <50% of bone marrow nucleated cells are plasma cells; otherwise platelet count >50×109/L (transfusions are not permitted to achieve this minimum platelet count).
   6. Women who are partners of men and of childbearing potential must be practicing one of the following methods of birth control: subcutaneous hormonal implant, levonorgestrel-releasing intra-uterine system, medroxyprogesterone acetate depot, tubal sterilization, ovulation inhibitory progesterone only pills, or sexual intercourse with a vasectomized male partner (vasectomy must be confirmed by 2 negative semen analyses). Or women will commit to absolute and continuous abstinence confirmed to her physician on a monthly basis. Contraception will start 4 weeks before the start of therapy, during therapy including dose interruptions, for 4 weeks after discontinuation of thalidomide and for 4 months after discontinuation of DARZALEX® (daratumumab).
   7. A woman of childbearing potential must have 2 negative serum or urine pregnancy tests at Screening, first within 10 to 14 days prior to dosing and the second within 24 hours prior to dosing.
   8. Each subject (or their legally acceptable representative) must sign an informed consent form (ICF) indicating that he or she understands the purpose of and procedures required for the study and are willing to participate in the study. Subject must be willing and able to adhere to the prohibitions and restrictions specified in this protocol.

### Exclusion Criteria

Any potential subject who meets any of the following criteria will be excluded from participating in the study.

1. Subject has received DARZALEX® (daratumumab) or other anti-CD38 therapies previously.
2. Subject has a diagnosis of primary amyloidosis, monoclonal gammopathy of undetermined significance, smoldering multiple myeloma, or solitary plasmacytoma. Monoclonal gammopathy of undetermined significance is defined by presence of serum M-protein <3 g/dL; absence of lytic bone lesions, anemia, hypercalcemia, and renal insufficiency related to the M-protein; and (if determined) proportion of plasma cells in the bone marrow of 10% or less. Smoldering multiple myeloma is defined as asymptomatic multiple myeloma with absence of related organ or tissue impairment (ROTI) end organ damage.
3. Subject has a diagnosis of Waldenström's macroglobulinemia, or other conditions in which IgM M-protein is present in the absence of a clonal plasma cell infiltration with lytic bone lesions.
4. Subject has prior or current systemic therapy or SCT for any plasma cell dyscrasia, with the exception of an emergency use of a short course (equivalent of dexamethasone 40 mg/day for a maximum 4 days) of corticosteroids before treatment.
5. Subject has peripheral neuropathy or neuropathic pain Grade 2 or higher, as defined by the National Cancer Institute Common Terminology Criteria for Adverse Events (NCI CTCAE) Version 4.
6. Subject has had any prior or concurrent invasive malignancy (other than multiple myeloma) within 10 years of study start except adequately treated basal cell or squamous cell carcinoma of the skin, carcinoma in situ of the cervix, localized prostate adenocarcinoma diagnosed ≥3 years and without evidence of biochemical failure, or other cancer for which the subject has undergone potentially curative therapy and has no evidence of that disease for ≥10 years.
7. Subject has had radiation therapy within 14 days of C1D1.
8. Subject has had plasmapheresis within 28 days of C1D1.
9. Subject is exhibiting clinical signs of meningeal involvement of multiple myeloma.
10a) Subject has known chronic obstructive pulmonary disease (COPD) with a Forced Expiratory Volume in 1 second (FEV1)<50% of predicted normal. Note that FEV1testing is required for patients suspected of having COPD and subjects must be excluded if FEV1<50% of predicted normal.
10b) Subject has known moderate or severe persistent asthma within the past 2 years or currently has uncontrolled asthma of any classification. (Note that subjects who currently have controlled intermittent asthma or controlled mild persistent asthma are allowed in the study).
11. Subject is known to be seropositive for history of human immunodeficiency virus (HIV) or known to have active hepatitis B or hepatitis C.
12. Subject has any concurrent medical or psychiatric condition or disease (e.g., active systemic infection, uncontrolled diabetes, acute diffuse infiltrative pulmonary disease) that is likely to interfere with the study procedures or results, or that in the opinion of the investigator, would constitute a hazard for participating in this study.
13. Subject has clinically significant cardiac disease, including:
myocardial infarction within 1 year before randomization, or an unstable or uncontrolled disease/condition related to or affecting cardiac function (e.g., unstable angina, congestive heart failure, New York Heart Association Class III-IV
uncontrolled cardiac arrhythmia (NCI CTCAE Version 4 Grade ≥2) or clinically significant ECG abnormalities
screening 12-lead ECG showing a baseline QT interval as corrected by Fridericia's formula (QTcF) >470 msec
   14. Subject has known allergies, hypersensitivity, or intolerance to boron or mannitol, corticosteroids, monoclonal antibodies or human proteins, or their excipients (refer to the Investigator's Brochure) or known sensitivity to mammalian-derived products. Or subject has known hypersensitivity to thalidomide.
   15. Subject has plasma cell leukemia (according to WHO criterion: ≥20% of cells in the peripheral blood with an absolute plasma cell count of more than 2×109/L) or POEMS syndrome (polyneuropathy, organomegaly, endocrinopathy, monoclonal protein, and skin changes).
   16. Subject is known or suspected of not being able to comply with the study protocol (e.g., because of alcoholism, drug dependency, or psychological disorder). Subject has any condition for which, in the opinion of the investigator, participation would not be in the best interest of the subject (e.g., compromise the well-being) or that could prevent, limit, or confound the protocol-specified assessments.
   17. Subject is a woman who is pregnant, or breast-feeding, or planning to become pregnant while enrolled in this study or within 4 months after the last dose of any component of the treatment regimen. Or, subject is a man who plans to father a child while enrolled in this study or within 4 months after the last dose of any component of the treatment regimen.
   18. Subject has had major surgery within 2 weeks before randomization or will not have fully recovered from surgery, or has surgery planned during the time the subject is expected to participate in the study. Kyphoplasty or Vertebroplasty is not considered major surgery.
   19. Subject has received an investigational drug (including investigational vaccines) or used an invasive investigational medical device within 4 weeks before randomization or is currently enrolled in an interventional investigational study.
   20. Subject has contraindications to the use of any components of the backbone treatment regimens, per local prescribing information.
   21. Incidence of gastrointestinal disease that may significantly alter the absorption of oral drugs.
   22. Subjects unable or unwilling to undergo antithrombotic prophylactic treatment.

### Dosage and Administration

DARZALEX® (daratumumab) (16 mg/kg) will be administered by intravenous (IV) infusion once every week for 8 weeks (VTD induction Cycle 1-2), then every 2 weeks for the remaining induction cycles and consolidation cycles based on treatment assignment. Following subsequent re randomization, subjects assigned to the DARZALEX® (daratumumab) maintenance arm will receive DARZALEX® (daratumumab) (16 mg/kg) once every 8 weeks until documented disease progression (limited to a maximum duration of 2 years).

Subjects will receive 1.3 mg/m² bortezomib as a subcutaneous (SC) injection twice a week (Days 1, 4, 8, and 11) for four 28-day induction cycles (Cycles 1 to 4), and two consolidation cycles (Cycles 5 and 6), with an option to change the schedule from twice a week to once a week, should toxicity be encountered. Cycles will remain 28 days in length regardless of injection interval.

Thalidomide will be administered PO at 100 mg daily for 4×28 day induction cycles and 2×28-day consolidation cycles. Thalidomide should be taken as a single dose at bedtime, to reduce the impact of somnolence. Thalidomide can be taken with or without food.

Dexamethasone will be administered twice a week (Days 1, 2, 8, 9, 15, 16, 22 and 23) at 40 mg during Cycles 1 and 2. In Cycles 3 and 4, dexamethasone will be administered at 40 mg on Days 1-2 and 20 mg on Days 8, 9, 15 and 16. Dexamethasone 20 mg will be administered in Cycles 5 and 6 on Days 1, 2, 8, 9, 15 and 16. In the maintenance phase, dexamethasone 20 mg will be administered as pre-medication on DARZALEX® (daratumumab) infusion days. On DARZALEX® (daratumumab) infusion days, dexamethasone may be administered intravenously 1 hour before the DARZALEX® (daratumumab) infusion. On days when DARZALEX® (daratumumab) is not administered, dexamethasone is administered PO. Dexamethasone tablets are to be taken with or immediately after a meal or snack, preferably in the morning.

### Study Part 1

### Induction Treatment

Subjects will receive up to 4×28 day cycles of VTD induction therapy as described. Subjects in Arm B will receive DARZALEX® (daratumumab) in addition to VTD. Efficacy will be assessed at the start of each cycle.

### Post Induction Efficacy Assessment (End of Cycle 4)

A bone marrow biopsy/aspirate (preferably both but morphologic review of the aspirate smear may be done if a core biopsy is not available) will be performed at the end of Cycle 4 to determine the plasma cell burden. The time window for this assessment is +/−3 days. As long as disease progression is not observed, subjects may proceed to stem cell mobilization. Subjects who cannot proceed to stem cell mobilization at this timepoint, based on investigator discretion or institutional practice, will be withdrawn from treatment. Subjects with disease progression will also be withdrawn from treatment. These subjects will enter the follow-up phase.

### Mobilization and Harvesting Stem Cells

Stem cell mobilization will be performed using cyclophosphamide (recommended dose of 3 g/m²) and G-CSF after Cycle 4 and stem cells will be harvested based on response to mobilization. The use of Plerixafor is permitted per institutional practice in case of failure. In case of subsequent of failure with Plerixafor bone marrow harvest may be performed. Sufficient stem cells should be harvested to enable multiple transplants in accordance with institutional standards.

An assessment of the efficiency of mobilization/harvesting will be recorded. During the transplant period (that is to say the period from the first day of hospitalization to the day before C5D1), neutropenia and thrombocytopenia resulting from bone marrow aplasia will not be recorded as AEs in the eCRF. Only the following have to be recorded in the eCRF:

any evolution of ongoing AE

any new AE related, or that appears to be related, to DARZALEX® (daratumumab)

any new infection from at least grade 3

any new oral mucositis from at least grade 3.

### Conditioning (Melphalan)

Subjects will receive melphalan 200 mg/m² as conditioning therapy over a period of 24 to 48 hours.

### Transplant

Subjects will have a single re-infusion of stem cells 24-48 hours after high-dose melphalan (+permitted tolerance)

### Engraftment/Recovery (Day 1-60 Post ASCT)

Subjects will be monitored for successful engraftment; support therapy will be administered according to institutional/study group standards.

### Consolidation (30-60 Days)

Consolidation therapy may commence when engraftment is complete and when in the opinion of the investigator the subject is fit enough to tolerate subsequent systemic therapy (30-60 days post ASCT). Subjects will receive a further 2×28-day cycles of VTD. Subjects randomly assigned to Arm B will receive DARZALEX® (daratumumab) and VTD. Efficacy will be assessed at the start of each cycle.

### Post Consolidation Efficacy Assessment (Day 100 Post ASCT)

Subjects will be assessed for efficacy for the primary endpoint at Day 100 post ASCT. If subjects are still receiving consolidation therapy at Day 100, the assessment of efficacy should be performed immediately upon completion of consolidation therapy. The time window for this assessment is +/−3 days from Day 100 or from end of consolidation therapy, as applicable.

### Study Part 2

Randomization to Maintenance/Observation Phase (Continuing from End of Last Consolidation Cycle, 50 Days)

Subjects who complete consolidation and attain a minimum of a partial response or better according to the IMWG criteria will be eligible for re-randomization to either DARZALEX® (daratumumab) maintenance or observation. Subjects who are not eligible for re-randomization will enter the Follow-up Phase.

The maximum time period between the end of the last consolidation cycle and second randomization should be no more than 50 days. Any assigned treatment should be initiated no later than 10 days after the second-randomization in to part 2 of the study.

All subjects will be assessed for efficacy every 8 weeks after re-randomization, additional assessments of MRD status will also be performed for patients who achieve at least VGPR (weeks 25, 52, 105 or whenever it will be necessary).

### End-of-Treatment Visit

Subjects will be treated for the maximal allowed treatment duration (or until disease progression, unacceptable toxicity, or for the other reasons). Unless a subject withdraws consent for study participation, or is lost to follow-up, an End-of-Treatment Visit is to be scheduled 30 days after the last dose of all components of the treatment regimen have been discontinued, or as soon as possible before the start of subsequent therapy. Every effort should be made to conduct the End-of-Treatment Visit before the subject starts subsequent therapy. If a subject is unable to return to the site for the End-of-Treatment Visit, then the subject should be contacted to collect adverse events and concomitant therapies that occur within 30 days after the last dose of any component of the treatment regimen.

### Follow-Up Phase

The Follow-up Phase will begin once a subject permanently discontinues treatment with study medications, except for subjects randomized to Arm A (observation) in the Maintenance Phase. These subjects will enter the Follow-up Phase 2 years after the second randomization or upon disease progression, withdrawal of consent, or start of new anticancer therapy, whichever is earliest.

For all subjects who complete or discontinue study drug without disease progression, disease evaluations should continue to be performed every 8 weeks until documented disease progression.

Thereafter subsequent anticancer treatment and response to treatment including date of subsequent progression (PFS2) will be recorded and survival status will be obtained.

In accordance with the 2011 IMWG consensus recommendations for the purposes of the study a line of therapy is defined as one or more cycles of a planned treatment program. The planned treatment approach of induction therapy followed by autologous stem cell transplantation, consolidation, and where applicable maintenance is considered one line of therapy.

A new line of therapy starts when a planned course of therapy is modified to include other treatment agents (alone or in combination) as a result of a disease progression, relapse, or toxicity.

### Safety Evaluations

Safety evaluations will include adverse event monitoring, physical examinations, electrocardiogram (ECGs) monitoring, clinical laboratory parameters (hematology and chemistry), vital sign measurements, and ECOG performance status.

### Biomarker Evaluations

Biomarkers will focus on the evaluation of MRD in bone marrow aspirates and on the assessment of clinical efficacy in high-risk molecular subgroups.

### Immunogenicity Evaluations

Samples to assess the generation of antibodies to DARZALEX® (daratumumab) (immunogenicity) and associated serum daratumumab concentration levels will be obtained from all subjects according to the Time and Events Schedule.

### Efficacy Evaluations

Assessment of tumor response and disease progression will be conducted in accordance with the IMWG response criteria. Efficacy evaluations will include measurements of tumor burden/residual disease, myeloma proteins, bone marrow examinations, skeletal surveys, extramedullary plasmacytomas, and serum calcium corrected for albumin.

### Statistical Methods

### Analysis Population

Analysis of primary and secondary efficacy variables will be based on the intent-to-treat (ITT) population, which includes all subjects randomized in the first randomization. In addition, maintenance-specific analyses will use the maintenance-specific intent-to-treat population (ITT-m), which will include all subjects who are randomized in the second randomization.

All safety analyses will be based on the safety analysis set. The safety population will be defined separately for the induction/ASCT/consolidation and maintenance stages. These populations will include all subjects randomized at each stage who received at least 1 dose of study drugs at the respective stage.

### Efficacy Analyses

The primary comparison of the 2 randomized induction/consolidation treatments will be made with respect to sCR rate using the Cochran-Mantel-Haenszel chi square test in the ITT population. A Mantel-Haenszel odds ratio, along with its 2-sided 95% confidence interval, will be calculated. All binary secondary endpoints for the induction/ASCT/consolidation stage will be analyzed similarly as the primary endpoint (sCR rate).

The statistical comparison between the 2 induction regimens with respect to PFS from the first randomization will need to take into consideration subsequent maintenance assignment (daratumumab maintenance or observation). A usual “as-randomized”-type of intent-to-treat analysis that compares the 2 induction treatments with respect to PFS without considering maintenance treatment has been shown to produce potentially biased estimates of treatment effects. As such, 2 appropriate intent-to-treat (ITT)-type of induction comparisons of particular interest, 1 specific to each maintenance treatment (DARZALEX® (daratumumab) maintenance or observation), will be conducted:
DARZALEX® (daratumumab)+VTD (D-VTD) induction/consolidation followed by DARZALEX® (daratumumab) maintenance vs. VTD induction/consolidation followed by DARZALEX® (daratumumab) maintenance, and
DARZALEX® (daratumumab)+VTD (D-VTD) induction/consolidation followed by observation vs. VTD induction/consolidation followed by observation

For each of the 2 comparisons, the analysis will include any subjects who are randomized in the first randomization and are then subsequently randomized to the specific maintenance treatment as well as those subjects who are randomized in the first randomization but are not randomized in the second randomization. A stratified Cox regression analysis with inverse probability weighting will be performed (Lokhnygina and Helterbrand, Biometrics 63:422-428, 2007), which yields unbiased estimates of treatment effects and maintains Type I error rate. The overall comparison of induction treatments will be made treating these 2 comparisons as 2 strata with the variance estimated using the robust variance estimator (the sandwich estimate). These 3 comparisons will all be tested with the significance level of 0.05 (2-sided) following the closed testing procedure. Essentially, the statistical significance is established for each of the 2 maintenance-specific comparisons if both itself and the overall induction comparison are significant at the 2-sided level of 0.05. Other time-to-event endpoints, except for duration of response, will be analyzed similarly. Duration of response will be presented descriptively using the weighted Kaplan-Meier estimates by Miyahara and Wahed, Stat Methods 10:2581-2591, 2010).

The primary comparison of the 2 randomized maintenance treatments (DARZALEX® (daratumumab) maintenance and observation) will be made with respect to PFS from the second randomization using a stratified log-rank test in the ITT-m population. The Kaplan-Meier method will be used to estimate the distribution of PFS from the second randomization for each treatment. The treatment effect (hazard ratio) and its 2-sided 95% confidence intervals are to be estimated using a stratified Cox regression model with maintenance treatment as the sole explanatory variable. In addition, the interaction between induction/consolidation and maintenance will be tested at a 2-sided significance level of 0.05 by a stratified Cox regression model that includes the interaction term between maintenance treatment and induction/consolidation treatment. All secondary time-to-event endpoints in the maintenance stage will be analyzed similarly as for the primary endpoint (PFS from the second randomization).

The comparison of the 2 randomized maintenance arms on binary secondary endpoints will be made using the Cochran-Mantel-Haenszel chi square test in the population of all subjects that are randomized in the second randomization. The observed rate of the binary outcome will be provided along its 2-sided 95% CIs. A Mantel-Haenszel odds ratio, along with its 2-sided 95% confidence interval, will be calculated.

### Safety Analyses

In general, adverse events that occurred during the induction/consolidation and maintenance stages will be summarized separately. Treatment-emergent adverse events for each stage will be defined as events that occur or worsen after administration of the first dose of during that stage and through 30 days after the last dose of study drug in that stage and before the next phase of treatment begins. Adverse events will be summarized by system organ class and preferred terms, NCI toxicity grade, and by action taken with study treatment.

Summaries, listings, datasets, or subject narratives will be provided, as appropriate, for those subjects who die, who discontinue treatment due to an adverse event, or who experience a severe or a serious adverse event. These will be provided using the same formats as those used for adverse events.

### Patient Reported Outcomes

To measure functional status, well-being, and symptoms, the EORTC QLQ-C30 and the EQ-SD-5L instruments will be used. Both questionnaires will be completed during visits before any other study procedures scheduled for the same day.

The EORTC QLQ-C30 includes 30 items resulting in 5 functional scales (physical functioning, role functioning, emotional functioning, cognitive functioning, and social functioning), 1 Global Health Status scale, 3 symptom scales (fatigue, nausea and vomiting, and pain), and 6 single items (dyspnea, insomnia, appetite loss, constipation, diarrhea, and financial difficulties). The recall period is 1 week (the past week). The EORTC QLQ-C30 has been widely used among cancer patients. Scores are transformed to a 0 to 100 scale. Administration time is approximately 11 minutes. Reliability, validity, and clinically meaningful change have been demonstrated in patients with multiple myeloma. The focus of the PRO assessment will be the global health scale which is designated as a secondary endpoint. The remaining domains are included as exploratory endpoints.

The EQ-5D-5L is a generic measure of health status. For purposes of this study, the EQ-5D-5L will be used to generate utility scores for use in cost effective analyses. The EQ-5D-5L is a 5-item questionnaire that assesses 5 domains including mobility, self-care, usual activities, pain/discomfort and anxiety/depression plus a visual analog scale rating “health today” with anchors ranging from 0 (worst imaginable health state) to 100 (best imaginable health state). The scores for the 5 separate questions are categorical and are cannot be analyzed as cardinal numbers. However, the scores for the 5 dimensions are used to compute a single utility score ranging from zero (0.0) to 1 (1.0) representing the general health status of the individual.

### Endpoints

### Part 1: Induction/ASCT/Consolidation Phase

### Primary Endpoint

Stringent Complete Response (sCR), by end of consolidation therapy, defined as the percentage of subjects achieving CR in addition to having a normal serum FLC ratio and an absence of clonal cells in bone marrow by immunohistochemistry, immunofluorescence or 2- to 4-color flow cytometry. Subjects who demonstrate all criteria for sCR, but have confirmed DARZALEX® (daratumumab) interference on SPEP and IFE, will be considered sCR.

Major Secondary Endpoints
PFS (from first randomization), defined as time from the initial randomization to either confirmed progressive disease (PD) per the IMWG criteria or death, whichever comes first. It is noted that the PFS events (PD or death) may include those that occur in the maintenance phase.
Time to progression (TTP) (from first randomization), defined as time from the initial randomization to confirmed progressive disease (PD) per the IMWG criteria, or death due to progressive disease, whichever occurs first. It is noted that the events (PD or death due to PD) may include those that occur in the maintenance phase.
Post-ASCT/consolidation CR rate, defined as the proportion of subjects who have achieved CR or better by the end of consolidation per the IMWG criteria.
Post-ASCT/consolidation MRD negative rate, defined as the proportion of subjects who have achieved MRD negative status by the end of consolidation per the IMWG criteria.
Post-induction sCR rate, defined as the proportion of subjects who have achieved sCR prior to high-dose therapy/ASCT per the IMWG criteria.
PFS2 (from first randomization), defined as the time from initial randomization to time of subsequent progression on next-line of therapy after disease progression on study treatment.
OS (from first randomization), measured from the date of initial randomization to the date of the subject's death. If the subject is alive or the vital status is unknown, then the subject's data will be censored at the date the subject was last known to be alive.

### Other Secondary Endpoints

Post-induction overall response rate (ORR) and rate of VGPR or better, defined as the proportions of subjects who have achieved PR or better by the end of induction per the IMWG criteria.
Duration of CR and sCR will be calculated from the date of the initial documentation of a CR or sCR to the date of the first documented evidence of relapse of CR or disease progression, as defined in the IMWG criteria, whichever occurs first. For subjects who have not relapsed nor progressed, data will be censored at the last disease evaluation.
Impact of D-VTD compared to VTD on patient-reported perception of global health

### Part 2: Maintenance Phase

### Primary Endpoint

Progression Free Survival (PFS) post completion of maintenance therapy, defined as the duration from the date of re-randomization to either progressive disease, according to the IMWG criteria, or death, whichever occurs first.

### Major Secondary Endpoints

TTP (from second randomization), defined as time from the second randomization to confirmed progressive disease (PD) per the IMWG criteria, or death due to progressive disease, whichever occurs first.
Overall CR rate, defined as the proportion of subjects who have achieved CR or better during the study per the IMWG criteria.
Overall MRD negative rate, defined as the proportion of subjects who have achieved MRD negative status by the end of study.
PFS2 (from second randomization), defined as the time from the second randomization to time of subsequent progression on next-line of therapy after disease progression on study treatment.
OS (from second randomization), defined as the time from the second randomization to the date of death.

### Other Secondary Endpoints

Rate of improved response during maintenance, defined as the proportion of subjects who have achieved a better category of response during maintenance compared to the response status at the end of consolidation (up to the second randomization). This is to be evaluated among the group of subjects who not achieved sCR by the second randomization.
Rate of MRD negative conversion during maintenance, defined as the proportion of subjects who have achieved de novo MRD negative status during maintenance.
ORR rate, defined as the proportion of subjects who have achieved PR or better by the end of study.

### Example 2: Phase 3 Randomized Study of DARZALEX® (Daratumumab) in Combination with Bortezomib, Thalidomide, and Dexamethasone (D-VTD) Versus VTD in Transplant-Eligible Newly Diagnosed Multiple Myeloma: Part 1 CASSIOPEIA Results

### Methods

In Part 1, transplant-eligible newly diagnosed multiple myeloma (NDMM) patients 18-65 years were randomized 1:1 to VTD (6 28-day cycles [C; 4 pre-ASCT induction, 2 post-ASCT consolidation] of V 1.3 mg/m² SC BIW Week [W] 1-2; T 100 mg PO QD; d 40-80 mg/week PO or IV W 1-4 C 1-2, W 1-3 C 3-6) ±DARZALEX® (daratumumab) (16 mg/kg IV QW C 1-2, Q2W C 3-6). Melphalan 200 mg/m² was pre-ASCT high-dose therapy. The primary endpoint was post-consolidation stringent complete response (sCR) rate assessed at Day 100 post-ASCT. Part 2 (maintenance) is ongoing. CASSIOPEIA study design is described in Example 1. MRD analyses were performed on bone marrow aspirates after induction by multiparametric flow cytometry (MFC; 10⁻⁵ sensitivity threshold).

### Results

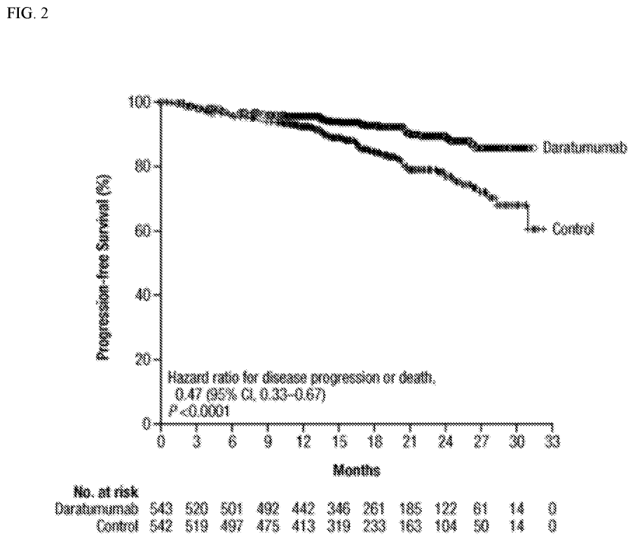

A cohort of 1085 patients (D-VTD, 543; VTD, 542) was randomized. The Day 100 post-ASCT sCR rate was significantly higher for D-VTD versus VTD (28.9% vs 20.3%; P=0.0010; Table 2). At 18.8-months median follow-up, progression-free survival (PFS) from first randomization favored D-VTD with hazard ratio (HR) 0.47 (95% CI, 0.33-0.67; P<0.0001; FIG. 2). With median PFS not reached in either arm, 18-month PFS rates were 92.7% versus 84.6% for D-VTD versus VTD. Rates of ≥CR, ≥VGPR, and MRD negativity supported sCR results (Table 2). Overall survival was immature with 46 deaths on study (D-VTD, 14; VTD, 32; HR, 0.43; 95% CI, 0.23-0.80).

The most common (≥10%) grade 3/4 treatment-emergent adverse events (D-VTD/VTD) were neutropenia (27.6%/14.7%), lymphopenia (17.0%/9.7%), stomatitis (12.7%/16.4%), and thrombocytopenia (11.0%/7.4%). In the D-VTD arm, infusion-related reactions occurred in 35.4% of patients.

### Conclusions

D-VTD in induction prior to and consolidation after ASCT improved depth of response (sCR, ≥CR, and MRD negativity) and PFS with acceptable safety. The favorable benefit-risk profile supports the use of D-VTD in transplant-eligible NDMM. CASSIOPEIA is the first study to demonstrate the clinical benefit of daratumumab plus standard of care in transplant-eligible NDMM.

TABLE 2

| | D-VTD, % | VTD, % | OR (95% CI) | P |
|---|---|---|---|---|
| sCR | 28.9 | 20.3 | 1.60 (1.21-2.12) | 0.0010 |
| ≥CR | 38.9 | 26.0 | 1.82 (1.40-2.36) | <0.0001 |
| ≥VGPR | 83.4 | 78.0 | 1.41 (1.04-1.92) | 0.0239 |
| MRD negative | 63.7 | 43.5 | 2.27 (1.78-2.90) | <0.0001 |
| ≥CR and MRD | 33.7 | 19.9 | 2.06 (1.56-2.72) | <0.0001 |
| negative | | | | |

### Example 3: Efficacy of DARZALEX® (Daratumumab) in Combination with Bortezomib, Thalidomide, and Dexamethasone (D-VTD) in Transplant-Eligible Newly Diagnosed Multiple Myeloma (TE NDMM) Based on Minimal Residual Disease (MRD) Status: Analysis of the CASSIOPEIA Trial

### Methods

Example 1 describes the trial design. In Part 1, TE NDMM patients were randomized 1:1 to 4 cycles of pre-ASCT induction and 2 cycles of post-ASCT consolidation with D-VTD or VTD alone. Landmark analyses of MRD were performed on bone marrow aspirates after induction by multiparametric flow cytometry (MFC; 10⁻⁵ sensitivity threshold) and after consolidation (at Day 100 post-ASCT) by MFC (1e) and next-generation sequencing (NGS; 10⁻⁶) for all patients, regardless of response.

### Results

1085 patients were randomized (D-VTD, 543; VTD, 542). The post-consolidation MRD-negative rate, regardless of response, was significantly higher for D-VTD vs VTD (63.7% vs 43.5%; P <0.0001). MRD-negative rates were consistent across patient subgroups, including ISS stage III disease or high-risk cytogenetics. In the D-VTD vs VTD arms, 33.7% vs 19.9% of patients achieved both ≥CR and MRD negativity (P<0.0001).

Multivariate analyses accounting for treatment arm (D-VTD vs VTD) and MRD negativity showed a PFS benefit in patients reaching MRD negativity regardless of response (HR, 0.31; 95% CI, 0.20-0.50; P<0.0001) and in patients with ≥CR (HR, 0.22; 95% CI, 0.10-0.48; P=0.0001). Based on these MRD multivariate analyses, D-VTD showed additional PFS benefit vs VTD regardless of response (HR, 0.48; 95% CI, 0.30-0.78; P=0.0028) and in s with ≥CR (HR, 0.46; 95% CI, 0.28-0.73; P=0.0011). Table 3 shows the MRD negativity rates in D-VTD and VTD arms.

### Conclusion

D-VTD induction and consolidation deepened response, with a significantly higher MRD-negative rate vs VTD alone. Deepened responses with D-VTD led to improved long-term outcomes, with MRD negativity associated with prolonged PFS in pts with TE NDMM.

TABLE 3

| | D-VTD, % | VTD, % | P |
|---|---|---|---|
| Post-induction | | | |
| MFC, 10⁻⁵ | 34.6 | 23.1 | <0.0001 |
| Post-consolidation | | | |
| MFC, 10⁻⁵ | 63.7 | 43.5 | <0.0001 |
| NGS, 10⁻⁶ | 39.1 | 22.8 | <0.0001 |

MFC: multiparameter flow cytometry
NGS: next-generation sequencing

TABLE 4
Sequence listing

| | Amino Acid Sequence | SEQ ID NO |
|---|---|---|
| HCDR1 | SFAMS | 1 |
| HCDR2 | AISGSGGGTYYADSVKG | 2 |
| HCDR3 | DKILWFGEPVFDY | 3 |
| LCDR1 | RASQSVSSYLA | 4 |
| LCDR2 | DASNRAT | 5 |
| LCDR3 | QQRSNWPPTF | 6 |
| VH | EVQLLESGGGLVQPGGSLRLSCAVSGFTFNSFAMSWVRQAP | 7 |
| | GKGLEWVSAISGSGGGTYYADSVKGRFTISRDNSKNTLYLQ | |
| | MNSLRAEDTAVYFCAKDKILWFGEPVFDYWGQGTLVTVSS | |
| VL | EIVLTQSPATLSLSPGERATLSCRASQSVSSYLAWYQQKPGQ | 8 |
| | APRLLIYDASNRATGIPARFSGSGSGTDFTLTISSLEPEDFAVY | |
| | YCQQRSNWPPTFGQGTKVEIK | |
| HC | EVQLLESGGGLVQPGGSLRLSCAVSGFTFNSFAMSWVRQAP | 9 |
| | GKGLEWVSAISGSGGGTYYADSVKGRFTISRDNSKNTLYLQ | |
| | MNSLRAEDTAVYFCAKDKILWFGEPVFDYWGQGTLVTVSS | |
| | ASTKGPSVFPLAPSSKSTSGGTAALGCLVKDYFPEPVTVSWN | |
| | SGALTSGVHTFPAVLQSSGLYSLSSVVTVPSSSLGTQTYICNV | |
| | NHKPSNTKVDKRVEPKSCDKTHTCPPCPAPELLGGPSVFLFP | |
| | PKPKDTLMISRTPEVTCVVVDVSHEDPEVKFNWYVDGVEV | |
| | HNAKTKPREEQYNSTYRVVSVLTVLHQDWLNGKEYKCKVS | |
| | NKALPAPIEKTISKAKGQPREPQVYTLPPSREEMTKNQVSLT | |
| | CLVKGFYPSDIAVEWESNGQPENNYKTTPPVLDSDGSFFLYS | |
| | KLTVDKSRWQQGNVFSCSVMHEALHNHYTQKSLSLSPGK | |
| LC | EIVLTQSPATLSLSPGERATLSCRASQSVSSYLAWYQQKPGQ | 10 |
| | APRLLIYDASNRATGIPARFSGSGSGTDFTLTISSLEPEDFAVY | |
| | YCQQRSNWPPTFGQGTKVEIKRTVAAPSVFIFPPSDEQLKSG | |
| | TASVVCLLNNFYPREAKVQWKVDNALQSGNSQESVTEQDS | |
| | KDSTYSLSSTLTLSKADYEKHKVYACEVTHQGLSSPVTKSF | |
| | NRGEC | |
| IgG1 | ASTKGPSVFPLAPSSKSTSGGTAALGCLVKDYFPEPVTVSWN | 11 |
| constant | SGALTSGVHTFPAVLQSSGLYSLSSVVTVPSSSLGTQTYICNV | |
| domain | NHKPSNTKVDKRVEPKSCDKTHTCPPCPAPELLGGPSVFLFP | |
| | PKPKDTLMISRTPEVTCVVVDVSHEDPEVKFNWYVDGVEV | |
| | HNAKTKPREEQYNSTYRVVSVLTVLHQDWLNGKEYKCKVS | |
| | NKALPAPIEKTISKAKGQPREPQVYTLPPSREEMTKNQVSLT | |
| | CLVKGFYPSDIAVEWESNGQPENNYKTTPPVLDSDGSFFLYS | |
| | KLTVDKSRWQQGNVFSCSVMHEALHNHYTQKSLSLSPGK | |

### Full Prescribing Information

### 1 INDICATIONS AND USAGE

DARZALEX is indicated for the treatment of adult patients with multiple myeloma:
in combination with lenalidomide and dexamethasone in newly diagnosed patients who are ineligible for autologous stem cell transplant and in patients with relapsed or refractory multiple myeloma who have received at least one prior therapy.
in combination with bortezomib, melphalan and prednisone in newly diagnosed patients who are ineligible for autologous stem cell transplant.
in combination with bortezomib, thalidomide, and dexamethasone in newly diagnosed patients who are eligible for autologous stem cell transplant
in combination with bortezomib and dexamethasone in patients who have received at least one prior therapy.
in combination with pomalidomide and dexamethasone in patients who have received at least two prior therapies including lenalidomide and a proteasome inhibitor.
as monotherapy, in patients who have received at least three prior lines of therapy including a proteasome inhibitor (PI) and an immunomodulatory agent or who are double-refractory to a PI and an immunomodulatory agent.

### 2 DOSAGE AND ADMINISTRATION

### 2.1 Recommended Dose and Schedule

Administer pre-infusion and post-infusion medications [see Dosage and Administration (2.2)].
Administer only as an intravenous infusion after dilution in 0.9% Sodium Chloride Injection, USP [see Dosage and Administration (2.4, 2.5)].
DARZALEX should be administered by a healthcare professional, with immediate access to emergency equipment and appropriate medical support to manage infusion reactions if they occur [see Warnings and Precautions (5.1)].
The DARZALEX dosing schedule in Table 5 is for combination therapy (4-week cycle regimens) and monotherapy as follows:
combination therapy with lenalidomide and low-dose dexamethasone for newly diagnosed patients ineligible for autologous stem cell transplant (ASCT) and in patients with relapsed/refractory multiple myeloma
   combination therapy with pomalidomide and low-dose dexamethasone for patients with relapsed/refractory multiple myeloma
   monotherapy for patients with relapsed/refractory multiple myeloma.

The recommended dose of DARZALEX is 16 mg/kg actual body weight administered as an intravenous infusion according to the following dosing schedule:

TABLE 5
DARZALEX Dosing Schedule in Combination With Lenalidomide
or Pomalidomide (4-Week Cycle Dosing Regimens) and Low-Dose
Dexamethasone and for Monotherapy

| Weeks | Schedule |
|---|---|
| Weeks 1 to 8 | weekly (total of 8 doses) |
| Weeks 9 to 24^{a} | every two weeks (total of 8 doses) |
| Week 25 onwards until disease | every four weeks |
| progression^{b} | |

^{a}First dose of the every-2-week dosing schedule is given at Week 9
^{b}First dose of the every-4-week dosing schedule is given at Week 25

For dosing instructions of combination agents administered with DARZALEX, see Clinical Studies (14) and manufacturer's prescribing information.

The DARZALEX dosing schedule in Table 6 is for combination therapy with bortezomib, melphalan and prednisone (6-week cycle regimen) for patients with newly diagnosed multiple myeloma ineligible for ASCT.

The recommended dose of DARZALEX is 16 mg/kg actual body weight administered as an intravenous infusion according to the following dosing schedule:

TABLE 6
DARZALEX Dosing Schedule in Combination With Bortezomib,
Melphalan and Prednisone ([VMP], 6-Week Cycle Dosing Regimen)

| Weeks | Schedule |
|---|---|
| Weeks 1 to 6 | weekly (total of 6 doses) |
| Weeks 7 to 54^{a} | every three weeks (total of 16 doses) |
| Week 55 onwards | every four weeks |
| until disease progression^{b} | |

^{a}First dose of the every-3-week dosing schedule is given at Week 7
^{b}First dose of the every-4-week dosing schedule is given at Week 55

For dosing instructions of combination agents administered with DARZALEX see Clinical Studies (14.1).

The DARZALEX dosing schedule in Table 7 is for combination therapy with bortezomib, thalidomide, and dexamethasone (4-week cycle regimen) for patients with newly diagnosed multiple myeloma eligible for ASCT.

The recommended dose of DARZALEX is 16 mg/kg actual body weight administered as an intravenous infusion according to the following dosing schedule:

TABLE 7
DARZALEX Dosing Schedule in Combination
With Bortezomib, Thalidomide and
Dexamethasone ([VTd]; 4-Week Cycle Dosing Regimen)

| Treatment | | |
|---|---|---|
| phase | Weeks | Schedule |
| Induction | Weeks 1 to 8 | weekly (total of 8 doses) |
| | Weeks 9 to 16^{a} | every two weeks (total of 4 doses) |

Stop for high dose chemotherapy and ASCT

| | | |
|---|---|---|
| Consolidation | Weeks 1 to 8^{b} | every two weeks (total of 4 doses) |

^{a}First dose of the every-2-week dosing schedule is given at Week 9
^{b}First dose of the every-2-week dosing schedule is given at Week 1 upon re-initiation of treatment following ASCT

For dosing instructions of combination agents administered with DARZALEX, see Clinical Studies (14.1) and the manufacturer's prescribing information.

The DARZALEX dosing schedule in Table 8 is for combination therapy with bortezomib and dexamethasone (3-week cycle regimen) for patients with relapsed/refractory multiple myeloma.

The recommended dose of DARZALEX is 16 mg/kg actual body weight administered as an intravenous infusion according to the following dosing schedule:

TABLE 8
DARZALEX Dosing Schedule With Bortezomib and
Dexamethasone (3-Week Cycle Dosing Regimen)

| Weeks | Schedule |
|---|---|
| Weeks 1 to 9 | weekly (total of 9 doses) |
| Weeks 10 to 24^{a} | every three weeks (total of 5 doses) |
| Week 25 onwards | every four weeks |
| until disease progression^{b} | |

^{a}First dose of the every-3-week dosing schedule is given at Week 10
^{b}First dose of the every-4-week dosing schedule is given at Week 25

For dosing instructions of combination agents administered with DARZALEX see Clinical Studies (14.2) and manufacturer's prescribing information.

### Missed DARZALEX Doses

If a planned dose of DARZALEX is missed, administer the dose as soon as possible and adjust the dosing schedule accordingly, maintaining the treatment interval.

### Infusion Rates and Management of Infusion Reactions

Administer DARZALEX infusion intravenously at the infusion rate described below in Table 9. Consider incremental escalation of the infusion rate only in the absence of infusion reactions.

To facilitate administration, the first prescribed 16 mg/kg dose at Week 1 may be split over two consecutive days i.e., 8 mg/kg on Day 1 and Day 2 respectively, see Table 9 below.

TABLE 9
Infusion Rates for DARZALEX (16 mg/kg) Administration

| | Dilution | | Initial rate | Rate | Maximum |
|---|---|---|---|---|---|
| | volume | | (first hour) | increment^{a} | rate |
| Week 1 Infusion | | | | | |
| Option 1 (Single | | | | | |
| dose infusion) | | | | | |
| Week 1 Day 1 | 1,000 | mL | 50 mL/ | 50 mL/hour | 200 mL/hour |
| (16 mg/kg) | | | hour | every hour | |
| Option 2 (Split | | | | | |
| dose infusion) | | | | | |
| Week 1 Day 1 | 500 | mL | 50 mL/ | 50 mL/hour | 200 mL/hour |
| (8 mg/kg) | | | hour | every hour | |
| Week 1 Day 2 | 500 | mL | 50 mL/ | 50 mL/hour | 200 mL/hour |
| (8 mg/kg) | | | hour | every hour | |
| Week 2 (16 mg/kg) | 500 | mL | 50 mL/ | 50 mL/hour | 200 mL/hour |
| infusion^{b} | | | hour | every hour | |
| Subsequent (Week | 500 | mL | 100 mL/ | 50 mL/hour | 200 mL/hour |

| | | |
|---|---|---|
| 3 onwards, | hour | every hour |
| 16 mg/kg) infusions^{c} | | |

^{a}Consider incremental escalation of the infusion rate only in the absence of infusion reactions.
^{b}Use a dilution volume of 500 mL for the 16 mg/kg dose only if there were no infusion reactions the previous week. Otherwise, use a dilution volume of 1,000 mL.
^{c}Use a modified initial rate (100 mL/hour) for subsequent infusions (i.e. Week 3 onwards) only if there were no infusion reactions during the previous infusion. Otherwise, continue to use instructions indicated in the table for the Week 2 infusion rate.

For infusion reactions of any grade/severity, immediately interrupt the DARZALEX infusion and manage symptoms. Management of infusion reactions may further require reduction in the rate of infusion, or treatment discontinuation of DARZALEX as outlined below [see Warnings and Precautions (5.1)].
Grade 1-2 (mild to moderate): Once reaction symptoms resolve, resume the infusion at no more than half the rate at which the reaction occurred. If the patient does not experience any further reaction symptoms, infusion rate escalation may resume at increments and intervals as clinically appropriate up to the maximum rate of 200 mL/hour (Table 9).
Grade 3 (severe): Once reaction symptoms resolve, consider restarting the infusion at no more than half the rate at which the reaction occurred. If the patient does not experience additional symptoms, resume infusion rate escalation at increments and intervals as outlined in Table 9. Repeat the procedure above in the event of recurrence of Grade 3 symptoms. Permanently discontinue DARZALEX upon the third occurrence of a Grade 3 or greater infusion reaction.
Grade 4 (life threatening): Permanently discontinue DARZALEX treatment.

### 2.2 Recommended Concomitant Medications

### Pre-Infusion Medication

Administer the following pre-infusion medications to reduce the risk of infusion reactions to all patients 1-3 hours prior to every infusion of DARZALEX:
Corticosteroid (long-acting or intermediate-acting)

Monotherapy:
Methylprednisolone 100 mg, or equivalent, administered intravenously. Following the second infusion, the dose of corticosteroid may be reduced (oral or intravenous methylprednisolone 60 mg).

Combination Therapy:
Administer 20 mg dexamethasone (or equivalent) prior to every DARZALEX infusion. When dexamethasone is the background regimen specific corticosteroid, the dexamethasone treatment dose will instead serve as pre-medication on DARZALEX infusion days [see Clinical Studies (14)].
Dexamethasone is given intravenously prior to the first DARZALEX infusion and oral administration may be considered prior to subsequent infusions. Additional background regimen-specific corticosteroids (e.g. prednisone) should not be taken on DARZALEX infusion days when patients receive dexamethasone (or equivalent) as a pre-medication.
Antipyretics (oral acetaminophen 650 to 1,000 mg)
Antihistamine (oral or intravenous diphenhydramine 25 to 50 mg or equivalent).

### Post-Infusion Medication

Administer post-infusion medication to reduce the risk of delayed infusion reactions to all patients as follows:

Monotherapy:
Administer oral corticosteroid (20 mg methylprednisolone or equivalent dose of an intermediate-acting or long-acting corticosteroid in accordance with local standards) on each of the 2 days following all DARZALEX infusions (beginning the day after the infusion).

Combination Therapy:
Consider administering low-dose oral methylprednisolone (less than or equal to 20 mg) or equivalent, the day after the DARZALEX infusion.
However, if a background regimen-specific corticosteroid (e.g. dexamethasone, prednisone) is administered the day after the DARZALEX infusion, additional post-infusion medications may not be needed [see Clinical Studies (14)].

In addition, for any patients with a history of chronic obstructive pulmonary disease, consider prescribing post-infusion medications such as short and long-acting bronchodilators, and inhaled corticosteroids. Following the first four infusions, if the patient experiences no major infusion reactions, these additional inhaled post-infusion medications may be discontinued.

### Prophylaxis for Herpes Zoster Reactivation

Initiate antiviral prophylaxis to prevent herpes zoster reactivation within 1 week after starting DARZALEX and continue for 3 months following treatment [see Adverse Reactions (6.1)].

### 2.3 Dose Modifications

No dose reductions of DARZALEX are recommended. Dose delay may be required to allow recovery of blood cell counts in the event of hematological toxicity [see Warnings and Precautions (5.3, 5.4)]. For information concerning drugs given in combination with DARZALEX, see manufacturer's prescribing information.

### 2.4 Preparation for Administration

DARZALEX is for single use only.

Prepare the solution for infusion using aseptic technique as follows:
Calculate the dose (mg), total volume (mL) of DARZALEX solution required and the number of DARZALEX vials needed based on patient actual body weight.
Check that the DARZALEX solution is colorless to pale yellow. Do not use if opaque particles, discoloration or other foreign particles are present.
Remove a volume of 0.9% Sodium Chloride Injection, USP from the infusion bag/container that is equal to the required volume of DARZALEX solution.
Withdraw the necessary amount of DARZALEX solution and dilute to the appropriate volume by adding to the infusion bag/container containing 0.9% Sodium Chloride Injection, USP as specified in Table 9 [see Dosage and Administration (2.1)]. Infusion bags/containers must be made of either polyvinylchloride (PVC), polypropylene (PP), polyethylene (PE) or polyolefin blend (PP+PE). Dilute under appropriate aseptic conditions. Discard any unused portion left in the vial.
Gently invert the bag/container to mix the solution. Do not shake.
Parenteral drug products should be inspected visually for particulate matter and discoloration prior to administration, whenever solution and container permit. The diluted solution may develop very small, translucent to white proteinaceous particles, as daratumumab is a protein. Do not use if visibly opaque particles, discoloration or foreign particles are observed.
Since DARZALEX does not contain a preservative, administer the diluted solution immediately at room temperature 15° C.−25° C. (59° F.−77° F.) and in room light. Diluted solution may be kept at room temperature for a maximum of 15 hours (including infusion time).
If not used immediately, the diluted solution can be stored prior to administration for up to 24 hours at refrigerated conditions 2° C.−8° C. (36° F.−46° F.) and protected from light. Do not freeze.

### 2.5 Administration

If stored in the refrigerator, allow the solution to come to room temperature. Administer the diluted solution by intravenous infusion using an infusion set fitted with a flow regulator and with an in-line, sterile, non-pyrogenic, low protein-binding polyethersulfone (PES) filter (pore size 0.22 or 0.2 micrometer). Administration sets must be made of either polyurethane (PU), polybutadiene (PBD), PVC, PP or PE.
Do not store any unused portion of the infusion solution for reuse. Any unused product or waste material should be disposed of in accordance with local requirements.
Do not infuse DARZALEX concomitantly in the same intravenous line with other agents.

### 3 DOSAGE FORMS AND STRENGTHS

DARZALEX is a colorless to pale yellow, preservative-free solution available as:

### Injection:

100 mg/5 mL (20 mg/mL) in a single-dose vial.
400 mg/20 mL (20 mg/mL) in a single-dose vial.

### 4 CONTRAINDICATIONS

DARZALEX is contraindicated in patients with a history of severe hypersensitivity (e.g. anaphylactic reactions) to daratumumab or any of the components of the formulation [see Warnings and Precautions (5.1) and Adverse Reactions (6.3)].

### 5 WARNINGS AND PRECAUTIONS

### 5.1 Infusion Reactions

DARZALEX can cause severe and/or serious infusion reactions including anaphylactic reactions. In clinical trials, approximately half of all patients experienced an infusion reaction. Most infusion reactions occurred during the first infusion and were Grade 1-2 [see Adverse Reactions (6.1)].

Infusion reactions can also occur with subsequent infusions. Nearly all reactions occurred during infusion or within 4 hours of completing DARZALEX. Prior to the introduction of post-infusion medication in clinical trials, infusion reactions occurred up to 48 hours after infusion.

Severe reactions have occurred, including bronchospasm, hypoxia, dyspnea, hypertension, laryngeal edema and pulmonary edema. Signs and symptoms may include respiratory symptoms, such as nasal congestion, cough, throat irritation, as well as chills, vomiting and nausea. Less common symptoms were wheezing, allergic rhinitis, pyrexia, chest discomfort, pruritus, and hypotension [see Adverse Reactions (6.1)].

Pre-medicate patients with antihistamines, antipyretics and corticosteroids. Frequently monitor patients during the entire infusion. Interrupt DARZALEX infusion for reactions of any severity and institute medical management as needed. Permanently discontinue DARZALEX therapy if an anaphylactic reaction or life-threatening (Grade 4) reaction occurs and institute appropriate emergency care. For patients with Grade 1, 2, or 3 reactions, reduce the infusion rate when re-starting the infusion [see Dosage and Administration (2.1)].

To reduce the risk of delayed infusion reactions, administer oral corticosteroids to all patients following DARZALEX infusions [see Dosage and Administration (2.2)]. Patients with a history of chronic obstructive pulmonary disease may require additional post-infusion medications to manage respiratory complications. Consider prescribing short- and long-acting bronchodilators and inhaled corticosteroids for patients with chronic obstructive pulmonary disease.

5.2 Interference with Serological Testing

Daratumumab binds to CD38 on red blood cells (RBCs) and results in a positive Indirect Antiglobulin Test (Indirect Coombs test). Daratumumab-mediated positive indirect antiglobulin test may persist for up to 6 months after the last daratumumab infusion. Daratumumab bound to RBCs masks detection of antibodies to minor antigens in the patient's serum [see References (15)]. The determination of a patient's ABO and Rh blood type are not impacted [see Drug Interactions (7.1)].

Notify blood transfusion centers of this interference with serological testing and inform blood banks that a patient has received DARZALEX. Type and screen patients prior to starting DARZALEX.

### 5.3 Neutropenia

DARZALEX may increase neutropenia induced by background therapy [see Adverse Reactions (6.1)].

Monitor complete blood cell counts periodically during treatment according to manufacturer's prescribing information for background therapies. Monitor patients with neutropenia for signs of infection. DARZALEX dose delay may be required to allow recovery of neutrophils. No dose reduction of DARZALEX is recommended. Consider supportive care with growth factors.

### 5.4 Thrombocytopenia

DARZALEX may increase thrombocytopenia induced by background therapy [see Adverse Reactions (6.1)].

Monitor complete blood cell counts periodically during treatment according to manufacturer's prescribing information for background therapies. DARZALEX dose delay may be required to allow recovery of platelets. No dose reduction of DARZALEX is recommended. Consider supportive care with transfusions.

5.5 Interference with Determination of Complete Response

Daratumumab is a human IgG kappa monoclonal antibody that can be detected on both, the serum protein electrophoresis (SPE) and immunofixation (IFE) assays used for the clinical monitoring of endogenous M-protein [see Drug Interactions (7.1)]. This interference can impact the determination of complete response and of disease progression in some patients with IgG kappa myeloma protein.

### 6 ADVERSE REACTIONS

The following clinically significant adverse reactions are also described elsewhere in the labeling:
Infusion reactions [see Warning and Precautions (5.1)].
Neutropenia [see Warning and Precautions (5.3)].
Thrombocytopenia [see Warning and Precautions (5.4)].

### 6.1 Clinical Trials Experience

Because clinical trials are conducted under widely varying conditions, adverse reaction rates observed in the clinical trials of a drug cannot be directly compared to rates in the clinical trials of another drug and may not reflect the rates observed in practice.

The safety data described below reflects exposure to DARZALEX (16 mg/kg) in 2,066 patients with multiple myeloma including 1,910 patients who received DARZALEX in combination with background regimens and 156 patients who received DARZALEX as monotherapy.

### Newly Diagnosed Multiple Myeloma Ineligible for Autologous Stem Cell Transplant

Combination Treatment with Lenalidomide and Dexamethasone (DRd)

Adverse reactions described in the table below reflect exposure to DARZALEX for a median treatment duration of 25.3 months (range: 0.1 to 40.44 months) for the daratumumab-lenalidomide-dexamethasone (DRd) group and median treatment duration of 21.3 months (range: 0.03 to 40.64 months) for the lenalidomide-dexamethasone group (Rd) in a Phase 3 active-controlled study MAIA. The most frequent (≥20%) adverse reactions were infusion reactions, diarrhea, constipation, nausea, peripheral edema, fatigue, back pain, asthenia, pyrexia, upper respiratory tract infection, bronchitis, pneumonia, decreased appetite, muscle spasms, peripheral sensory neuropathy, dyspnea and cough. Serious adverse reactions with a 2% greater incidence in the DRd arm compared to the Rd arm were pneumonia (DRd 15% vs Rd 8%), bronchitis (DRd 4% vs Rd 2%) and dehydration (DRd 2% vs Rd<1%).

TABLE 10
Adverse Reactions Reported in ≥10% of Patients and With at Least a 5% Greater
Frequency in the DRd Arm in MAIA

| | DRd (N = 364) | | | Rd (N = 365) | | |
|---|---|---|---|---|---|---|
| Body System | Any Grade | Grade 3 | Grade 4 | Any Grade | Grade 3 | Grade 4 |
| Adverse Reaction | (%) | (%) | (%) | (%) | (%) | (%) |
| Infusion reactions^{a} | 41 | 2 | <1 | 0 | 0 | 0 |

Gastrointestinal disorders

| | | | | | | |
|---|---|---|---|---|---|---|
| Diarrhea | 57 | 7 | 0 | 46 | 4 | 0 |
| Constipation | 41 | 1 | <1 | 36 | <1 | 0 |
| Nausea | 32 | 1 | 0 | 23 | 1 | 0 |
| Vomiting | 17 | 1 | 0 | 12 | <1 | 0 |

General disorders and administration site conditions

| | | | | | | |
|---|---|---|---|---|---|---|
| Peripheral edema^{b} | 41 | 2 | 0 | 33 | 1 | 0 |
| Fatigue | 40 | 8 | 0 | 28 | 4 | 0 |
| Asthenia | 32 | 4 | 0 | 25 | 3 | <1 |
| Pyrexia | 23 | 2 | 0 | 18 | 2 | 0 |
| Chills | 13 | 0 | 0 | 2 | 0 | 0 |

Infections and infestations

| | | | | | | |
|---|---|---|---|---|---|---|
| Upper respiratory tract | 52 | 2 | <1 | 36 | 2 | <1 |
| infection^{c} | | | | | | |
| Bronchitis^{d} | 29 | 3 | 0 | 21 | 1 | 0 |
| Pneumonia^{e} | 26 | 14 | 1 | 14 | 7 | 1 |
| Urinary tract infection | 18 | 2 | 0 | 10 | 2 | 0 |

Metabolism and nutrition disorders

| | | | | | | |
|---|---|---|---|---|---|---|
| Decreased appetite | 22 | 1 | 0 | 15 | <1 | <1 |
| Hyperglycemia | 14 | 6 | 1 | 8 | 3 | 1 |
| Hypocalcemia | 14 | 1 | <1 | 9 | 1 | 1 |

Musculoskeletal and connective tissue disorders

| | | | | | | |
|---|---|---|---|---|---|---|
| Back pain | 34 | 3 | <1 | 26 | 3 | <1 |
| Muscle spasms | 29 | 1 | 0 | 22 | 1 | 0 |

Nervous system disorders

| | | | | | | |
|---|---|---|---|---|---|---|
| Peripheral sensory neuropathy | 24 | 1 | 0 | 15 | 0 | 0 |
| Headache | 19 | 1 | 0 | 11 | 0 | 0 |
| Paresthesia | 16 | 0 | 0 | 8 | 0 | 0 |

Respiratory, thoracic and mediastinal disorders

| | | | | | | |
|---|---|---|---|---|---|---|
| Dyspnea^{f} | 32 | 3 | <1 | 20 | 1 | 0 |
| Cough^{g} | 30 | <1 | 0 | 18 | 0 | 0 |

Vascular disorders

| | | | | | | |
|---|---|---|---|---|---|---|
| Hypertension^{h} | 13 | 6 | <1 | 7 | 4 | 0 |

Key: D = daratumumab,
Rd = lenalidomide-dexamethasone.
^{a}Infusion reaction includes terms determined by investigators to be related to infusion, see section on Infusion Reactions below
^{b}Generalized edema, Gravitational edema, Edema, Peripheral edema, Peripheral swelling
^{c}Acute sinusitis, Bacterial rhinitis, Laryngitis, Metapneumovirus infection, Nasopharyngitis, Oropharyngeal candidiasis, Pharyngitis, Respiratory syncytial virus infection, Respiratory tract infection, Respiratory tract infection viral, Rhinitis, Rhinovirus infection, Sinusitis, Tonsillitis, Tracheitis, Upper respiratory tract infection, Viral pharyngitis, Viral rhinitis, Viral upper respiratory tract infection
^{d}Bronchiolitis, Bronchitis, Bronchitis viral, Respiratory syncytial virus bronchiolitis, Tracheobronchitis
^{e}Atypical pneumonia, Bronchopulmonary aspergillosis, Lung infection, Pneumocystis jirovecii infection, Pneumocystis jirovecii pneumonia, Pneumonia, Pneumonia aspiration, Pneumonia pneumococcal, Pneumonia viral, Pulmonary mycosis
^{f}Dyspnea, Dyspnea exertional
^{g}Cough, Productive cough
^{h}Blood pressure increased, Hypertension

Laboratory abnormalities worsening during treatment from baseline listed in Table 11.

TABLE 11
Treatment-Emergent Hematology Laboratory
Abnormalities in MAIA

| | DRd (N = 364) % | | | Rd (N = 365) % | | |
|---|---|---|---|---|---|---|
| | Any | Grade | Grade | Any | Grade | Grade |
| | Grade | 3 | 4 | Grade | 3 | 4 |
| Anemia | 47 | 13 | 0 | 57 | 24 | 0 |
| Thrombo- | 67 | 6 | 3 | 58 | 7 | 4 |
| cytopenia | | | | | | |
| Leukopenia | 90 | 30 | 5 | 82 | 20 | 4 |
| Neutropenia | 91 | 39 | 17 | 77 | 28 | 11 |
| Lymphopenia | 84 | 41 | 11 | 75 | 36 | 6 |

Key: D = daratumumab,
Rd = lenalidomide-dexamethasone.

Combination Treatment with Bortezomib, Melphalan and Prednisone

Adverse reactions described in Table 12 reflect exposure to DARZALEX for a median treatment duration of 14.7 months (range: 0 to 25.8 months) for the daratumumab, bortezomib, melphalan and prednisone (D-VMP) group, and median treatment duration of 12 months (range: 0.1 to 14.9 months) for the VMP group in a Phase 3 active-controlled study ALCYONE. The most frequent adverse reactions (≥20% with at least 5% greater frequency in the D-VMP arm) were infusion reactions, upper respiratory tract infection and edema peripheral. Serious adverse reactions with at least a 2% greater incidence in the D-VMP arm compared to the VMP arm were pneumonia (D-VMP 11% vs VMP 4%), upper respiratory tract infection (D-VMP 5% vs VMP 1%), and pulmonary edema (D-VMP 2% vs VMP 0%).

TABLE 12
Adverse Reactions Reported in ≥10% of Patients and With at
Least a 5% Greater Frequency in the D-VMP Arm in ALCYONE

| Body | | | | | | |
|---|---|---|---|---|---|---|
| System | D-VMP (N = 346) | | | VMP (N = 354) | | |
| Adverse | Any Grade | Grade 3 | Grade 4 | Any Grade | Grade 3 | Grade 4 |
| Reaction | (%) | (%) | (%) | (%) | (%) | (%) |
| Infusion | 28 | 4 | 1 | 0 | 0 | 0 |
| reactions^{a} | | | | | | |

General disorders and administration site conditions

| | | | | | | |
|---|---|---|---|---|---|---|
| Edema | 21 | 1 | <1 | 14 | 1 | 0 |
| peripheral^{b} | | | | | | |

Infections and infestations

| | | | | | | |
|---|---|---|---|---|---|---|
| Upper | 48 | 5 | 0 | 28 | 3 | 0 |
| respiratory | | | | | | |
| tract | | | | | | |
| infection^{c} | | | | | | |
| Pneumonia^{d} | 16 | 12 | <1 | 6 | 5 | <1 |

Respiratory, thoracic and mediastinal disorders

| | | | | | | |
|---|---|---|---|---|---|---|
| Cough^{e} | 16 | <1 | 0 | 8 | <1 | 0 |
| Dyspnea^{f} | 13 | 2 | 1 | 5 | 1 | 0 |

Vascular disorders

| | | | | | | |
|---|---|---|---|---|---|---|
| Hyper- | 10 | 4 | <1 | 3 | 2 | 0 |
| tension^{g} | | | | | | |

Key: D = daratumumab,
VMP = bortezomib-melphalan-prednisone
^{a}Infusion reaction includes terms determined by investigators to be related to infusion, see section on Infusion Reactions below.
^{b}edema peripheral, generalized edema, peripheral swelling
^{c}upper respiratory tract infection, bronchitis, bronchitis bacterial, epiglottitis, laryngitis, laryngitis bacterial, metapneumovirus infection, nasopharyngitis, oropharyngeal candidiasis, pharyngitis, pharyngitis streptococcal, respiratory syncytial virus infection, respiratory tract infection, respiratory tract infection viral, rhinitis, sinusitis, tonsillitis, tracheitis, tracheobronchitis, viral pharyngitis, viral rhinitis, viral upper respiratory tract infection.
^{d}pneumonia, lung infection, pneumonia aspiration, pneumonia bacterial, pneumonia pneumococcal, pneumonia streptococcal, pneumonia viral, and pulmonary sepsis
^{e}cough, productive cough
^{f}dyspnea, dyspnea exertional
^{g}hypertension, blood pressure increased

Laboratory abnormalities worsening during treatment from baseline listed in Table 13.

TABLE 13
Treatment-Emergent Hematology Laboratory
Abnormalities in ALCYONE

| | D-VMP (N = 346) % | | | VMP (N = 354) % | | |
|---|---|---|---|---|---|---|
| | Any | Grade | Grade | Any | Grade | Grade |
| | Grade | 3 | 4 | Grade | 3 | 4 |
| Anemia | 47 | 18 | 0 | 50 | 21 | 0 |
| Thrombo- | 88 | 27 | 11 | 88 | 26 | 16 |
| cytopenia | | | | | | |
| Neutropenia | 86 | 34 | 10 | 87 | 32 | 11 |
| Lymphopenia | 85 | 46 | 12 | 83 | 44 | 9 |

Key: D = daratumumab,
VMP = bortezomib-melphalan-prednisone

### Newly Diagnosed Multiple Myeloma Eligible for Autologous Stem Cell Transplant

Combination Treatment with Bortezomib, Thalidomide and Dexamethasone (DVTd)

Adverse reactions described in Table 14 reflect exposure to DARZALEX up to day 100 post-transplant in a Phase 3 active-controlled study CASSIOPEIA [see Clinical Studies (14.1)]. The median duration of induction/ASCT/consolidation treatment was 8.9 months (range: 7.0 to 12.0 months) for the DVTd group and 8.7 months (range: 6.4 to 11.5 months) for the VTd group. The most frequent adverse reactions (>20% with at least 5% greater frequency in the DVTd group) were infusion reactions, nausea, pyrexia, upper respiratory tract infection and bronchitis. Serious adverse reactions with a 2% greater incidence in the DVTd arm compared to the VTd arm were bronchitis (DVTd 2% vs VTd <1%) and pneumonia (DVTd 6% vs VTd 4%).

TABLE 14
Adverse Reactions Reported in ≥10% of Patients and With at Least a 5% Greater Frequency
in the DVTd Arm in CASSIOPEIA

| | DVTd (N = 536) | | | VTd (N = 538) | | |
|---|---|---|---|---|---|---|
| Body System | Any Grade | Grade 3 | Grade 4 | Any Grade | Grade 3 | Grade 4 |
| Adverse Reaction | (%) | (%) | (%) | (%) | (%) | (%) |
| Infusion reactions^{a} | 35 | 3 | <1 | 0 | 0 | 0 |

Gastrointestinal disorders

| | | | | | | |
|---|---|---|---|---|---|---|
| Nausea | 30 | 4 | 0 | 24 | 2 | <1 |
| Vomiting | 16 | 2 | 0 | 10 | 2 | 0 |

General disorders and administration site conditions

| | | | | | | |
|---|---|---|---|---|---|---|
| Pyrexia | 26 | 2 | <1 | 21 | 2 | 0 |

Infections and infestations

| | | | | | | |
|---|---|---|---|---|---|---|
| Upper respiratory tract | 27 | 1 | 0 | 17 | 1 | 0 |
| infection^{b} | | | | | | |
| Bronchitis^{c} | 20 | 1 | 0 | 13 | 1 | 0 |

Respiratory, thoracic and mediastinal disorders

| | | | | | | |
|---|---|---|---|---|---|---|
| Cough^{d} | 17 | 0 | 0 | 9 | 0 | 0 |

Vascular disorders

| | | | | | | |
|---|---|---|---|---|---|---|
| Hypertension | 10 | 4 | 0 | 5 | 2 | 0 |

Key: D = daratumumab,
VTd = bortezomib-thalidomide -dexamethasone.
^{a}Infusion reaction includes terms determined by investigators to be related to infusion, see section on Infusion Reactions below
^{b}Laryngitis, Laryngitis viral, Metapneumovirus infection, Nasopharyngitis, Oropharyngeal candidiasis, Pharyngitis, Respiratory syncytial virus infection, Respiratory tract infection, Respiratory tract infection viral, Rhinitis, Rhinovirus infection, Sinusitis, Tonsillitis, Tracheitis, Upper respiratory tract infection, Viral pharyngitis, Viral rhinitis, Viral upper respiratory tract infection
^{c}Bronchiolitis, Bronchitis, Bronchitis chronic, Respiratory syncytial virus bronchitis, Tracheobronchitis
^{d}Cough, Productive cough
Note:
Hematology laboratory related toxicities were excluded and reported separately in the table below

TABLE 15
Treatment-Emergent Hematology Laboratory
Abnormalities in CASSIOPEIA

| | DVTd (N = 536) % | | | VTd (N = 538) % | | |
|---|---|---|---|---|---|---|
| | Any | Grade | Grade | Any | Grade | Grade |
| | Grade | 3 | 4 | Grade | 3 | 4 |
| Anemia | 36 | 4 | 0 | 35 | 5 | 0 |
| Thrombocytopenia | 81 | 9 | 5 | 58 | 8 | 3 |
| Leukopenia | 82 | 14 | 10 | 57 | 6 | 9 |
| Neutropenia | 63 | 19 | 14 | 41 | 10 | 9 |
| Lymphopenia | 95 | 44 | 15 | 91 | 37 | 10 |

Key: D = daratumumab,
VTd = bortezomib-thalidomide -dexamethasone.

### Relapsed/Refractory Multiple Myeloma

Combination Treatment with Lenalidomide and Dexamethasone

Adverse reactions described in Table 16 reflect exposure to DARZALEX for a median treatment duration of 13.1 months (range: 0 to 20.7 months) for the daratumumab-lenalidomide-dexamethasone (DRd) group and median treatment duration of 12.3 months (range: 0.2 to 20.1 months) for the lenalidomide-dexamethasone (Rd) group in a Phase 3 active-controlled study POLLUX. The most frequent adverse reactions (≥20%) were infusion reactions, diarrhea, nausea, fatigue, pyrexia, upper respiratory tract infection, muscle spasms, cough and dyspnea. The overall incidence of serious adverse reactions was 49% for the DRd group compared with 42% for the Rd group. Serious adverse reactions with at least a 2% greater incidence in the DRd arm compared to the Rd arm were pneumonia (DRd 12% vs Rd 10%), upper respiratory tract infection (DRd 7% vs Rd 4%), influenza and pyrexia (DRd 3% vs Rd 1% for each).

Adverse reactions resulted in discontinuations for 7% (n=19) of patients in the DRd arm versus 8% (n=22) in the Rd arm.

TABLE 16
Adverse Reactions Reported in ≥10% of Patients and With at Least a 5% Greater
Frequency in the DRd Arm in POLLUX

| | DRd (N = 283) % | | | Rd (N = 281) % | | |
|---|---|---|---|---|---|---|
| Adverse Reaction | Any Grade | Grade 3 | Grade 4 | Any Grade | Grade 3 | Grade 4 |
| Infusion reactions^{a} | 48 | 5 | 0 | 0 | 0 | 0 |

Gastrointestinal disorders

| | | | | | | |
|---|---|---|---|---|---|---|
| Diarrhea | 43 | 5 | 0 | 25 | 3 | 0 |
| Nausea | 24 | 1 | 0 | 14 | 0 | 0 |
| Vomiting | 17 | 1 | 0 | 5 | 1 | 0 |

General disorders and administration site conditions

| | | | | | | |
|---|---|---|---|---|---|---|
| Fatigue | 35 | 6 | <1 | 28 | 2 | 0 |
| Pyrexia | 20 | 2 | 0 | 11 | 1 | 0 |

Infections and infestations

| | | | | | | |
|---|---|---|---|---|---|---|
| Upper respiratory | 65 | 6 | <1 | 51 | 4 | 0 |
| tract infection^{b} | | | | | | |

Musculoskeletal and connective tissue disorders

| | | | | | | |
|---|---|---|---|---|---|---|
| Muscle spasms | 26 | 1 | 0 | 19 | 2 | 0 |
| Nervous system disorders | | | | | | |
| Headache | 13 | 0 | 0 | 7 | 0 | 0 |

Respiratory, thoracic and mediastinal disorders

| | | | | | | |
|---|---|---|---|---|---|---|
| Cough^{c} | 30 | 0 | 0 | 15 | 0 | 0 |
| Dyspnea^{d} | 21 | 3 | <1 | 12 | 1 | 0 |

Key: D = daratumumab,
Rd = lenalidomide-dexamethasone.
^{a}Infusion reaction includes terms determined by investigators to be related to infusion, see section on Infusion Reactions below.
^{b}upper respiratory tract infection, bronchitis, sinusitis, respiratory tract infection viral, rhinitis, pharyngitis, respiratory tract infection, metapneumovirus infection, tracheobronchitis, viral upper respiratory tract infection, laryngitis, respiratory syncytial virus infection, staphylococcal pharyngitis, tonsillitis, viral pharyngitis, acute sinusitis, nasopharyngitis, bronchiolitis, bronchitis viral, pharyngitis streptococcal, tracheitis, upper respiratory tract infection bacterial, bronchitis bacterial, epiglottitis, laryngitis viral, oropharyngeal candidiasis, respiratory moniliasis, viral rhinitis, acute tonsillitis, rhinovirus infection
^{c}cough, productive cough, allergic cough
^{d}dyspnea, dyspnea exertional

Laboratory abnormalities worsening during treatment from baseline listed in Table 17.

TABLE 17
Treatment-Emergent Hematology Laboratory
Abnormalities in POLLUX

| | DRd (N = 283) % | | | Rd (N = 281) % | | |
|---|---|---|---|---|---|---|
| | Any | Grade | Grade | Any | Grade | Grade |
| | Grade | 3 | 4 | Grades | 3 | 4 |
| Anemia | 52 | 13 | 0 | 57 | 19 | 0 |
| Thrombocytopenia | 73 | 7 | 6 | 67 | 10 | 5 |
| Neutropenia | 92 | 36 | 17 | 87 | 32 | 8 |
| Lymphopenia | 95 | 42 | 10 | 87 | 32 | 6 |

Key: D = Daratumumab,
Rd = lenalidomide-dexamethasone.

Combination Treatment with Bortezomib and Dexamethasone

Adverse reactions described in Table 18 reflect exposure to DARZALEX for a median treatment duration of 6.5 months (range: 0 to 14.8 months) in the daratumumab-bortezomib-dexamethasone (DVd) group and median treatment duration of 5.2 months (range: 0.2 to 8.0 months) for the bortezomib-dexamethasone (Vd) group in a Phase 3 active-controlled study CASTOR. The most frequent adverse reactions (>20%) were infusion reactions, diarrhea, peripheral edema, upper respiratory tract infection, peripheral sensory neuropathy, cough and dyspnea. The overall incidence of serious adverse reactions was 42% for the DVd group compared with 34% for the Vd group. Serious adverse reactions with at least a 2% greater incidence in the DVd arm compared to the Vd arm were upper respiratory tract infection (DVd 5% vs Vd 2%), diarrhea and atrial fibrillation (DVd 2% vs Vd 0% for each).

Adverse reactions resulted in discontinuations for 7% (n=18) of patients in the DVd arm versus 9% (n=22) in the Vd arm.

TABLE 18
Adverse Reactions Reported in ≥10% of Patients and With at
Least a 5% Greater Frequency in the DVd Arm CASTOR

| Adverse | DVd (N = 243) % | | | Vd (N = 237) % | | |
|---|---|---|---|---|---|---|
| Reaction | Any Grade | Grade 3 | Grade 4 | Any Grade | Grade 3 | Grade 4 |
| Infusion | 45 | 9 | 0 | 0 | 0 | 0 |
| reactions^{a} | | | | | | |

Gastrointestinal disorders

| | | | | | | |
|---|---|---|---|---|---|---|
| Diarrhea | 32 | 3 | <1 | 22 | 1 | 0 |
| Vomiting | 11 | 0 | 0 | 4 | 0 | 0 |

General disorders and administration site conditions

| | | | | | | |
|---|---|---|---|---|---|---|
| Edema | 22 | 1 | 0 | 13 | 0 | 0 |
| peripheral^{b} | | | | | | |
| Pyrexia | 16 | 1 | 0 | 11 | 1 | 0 |

Infections and infestations

| | | | | | | |
|---|---|---|---|---|---|---|
| Upper | 44 | 6 | 0 | 30 | 3 | <1 |
| respiratory | | | | | | |
| tract | | | | | | |
| infection^{c} | | | | | | |

Nervous system disorders

| | | | | | | |
|---|---|---|---|---|---|---|
| Peripheral | 47 | 5 | 0 | 38 | 6 | <1 |
| sensory | | | | | | |
| neuropathy | | | | | | |

Respiratory, thoracic and mediastinal disorders

| | | | | | | |
|---|---|---|---|---|---|---|
| Cough^{d} | 27 | 0 | 0 | 14 | 0 | 0 |
| Dyspnea^{e} | 21 | 4 | 0 | 11 | 1 | 0 |

Key: D = daratumumab,
Vd = bortezomib-dexamethasone.
^{a}Infusion reaction includes terms determined by investigators to be related to infusion, see section on Infusion Reactions below.
^{b}edema peripheral, edema, generalized edema, peripheral swelling
^{c}upper respiratory tract infection, bronchitis, sinusitis, respiratory tract infection viral, rhinitis, pharyngitis, respiratory tract infection, metapneumovirus infection, tracheobronchitis, viral upper respiratory tract infection, laryngitis, respiratory syncytial virus infection, staphylococcal pharyngitis, tonsillitis, viral pharyngitis, acute sinusitis, nasopharyngitis, bronchiolitis, bronchitis viral, pharyngitis streptococcal, tracheitis, upper respiratory tract infection bacterial, bronchitis bacterial, epiglottitis, laryngitis viral, oropharyngeal candidiasis, respiratory moniliasis, viral rhinitis, acute tonsillitis, rhinovirus infection
^{d}cough, productive cough, allergic cough
^{e}dyspnea, dyspnea exertional

Laboratory abnormalities worsening during treatment are listed in Table 19.

TABLE 19
Treatment-Emergent Hematology Laboratory
Abnormalities in CASTOR

| | DVd (N = 243) % | | | Vd (N = 237) % | | |
|---|---|---|---|---|---|---|
| | Any | Grade | Grade | Any | Grade | Grade |
| | Grade | 3 | 4 | Grade | 3 | 4 |
| Anemia | 48 | 13 | 0 | 56 | 14 | 0 |
| Thrombocytopenia | 90 | 28 | 19 | 85 | 22 | 13 |
| Neutropenia | 58 | 12 | 3 | 40 | 5 | <1 |
| Lymphopenia | 89 | 41 | 7 | 81 | 24 | 3 |

Key: D = Daratumumab,
Vd = bortezomib-dexamethasone.

Combination Treatment with Pomalidomide and Dexamethasone

Adverse reactions described in Table 20 reflect exposure to DARZALEX, pomalidomide and dexamethasone (DPd) for a median treatment duration of 6 months (range: 0.03 to 16.9 months) in EQUULEUS. The most frequent adverse reactions (>20%) were infusion reactions, diarrhea, constipation, nausea, vomiting, fatigue, pyrexia, upper respiratory tract infection, muscle spasms, back pain, arthralgia, dizziness, insomnia, cough and dyspnea. The overall incidence of serious adverse reactions was 49%. Serious adverse reactions reported in ≥5% patients included pneumonia (7%). Adverse reactions resulted in discontinuations for 13% of patients.

TABLE 20
Adverse Reactions With Incidence ≥ 10% Reported in EQUULEUS

| Body System | DPd (N = 103) | | |
|---|---|---|---|
| Adverse Reaction | Any Grade (%) | Grade 3 (%) | Grade 4 (%) |
| Infusion reactions^{a} | 50 | 4 | 0 |

Gastrointestinal disorders

| | | | |
|---|---|---|---|
| Diarrhea | 38 | 3 | 0 |
| Constipation | 33 | 0 | 0 |
| Nausea | 30 | 0 | 0 |
| Vomiting | 21 | 2 | 0 |

General disorders and administration site conditions

| | | | |
|---|---|---|---|
| Fatigue | 50 | 10 | 0 |
| Pyrexia | 25 | 1 | 0 |
| Chills | 20 | 0 | 0 |
| Edema peripheral^{b} | 17 | 4 | 0 |
| Asthenia | 15 | 0 | 0 |
| Non-cardiac | 15 | 0 | 0 |
| chest pain | | | |
| Pain | 11 | 0 | 0 |

Infections and infestations

| | | | |
|---|---|---|---|
| Upper respiratory | 50 | 4 | 1 |
| tract infection^{c} | | | |
| Pneumonia^{d} | 15 | 8 | 2 |

Metabolism and nutrition disorders

| | | | |
|---|---|---|---|
| Hypokalemia | 16 | 3 | 0 |
| Hyperglycemia | 13 | 5 | 1 |
| Decreased appetite | 11 | 0 | 0 |

Musculoskeletal and connective tissue disorders

| | | | |
|---|---|---|---|
| Muscle spasms | 26 | 1 | 0 |
| Back pain | 25 | 6 | 0 |
| Arthralgia | 22 | 2 | 0 |
| Pain in extremity | 15 | 0 | 0 |
| Bone pain | 13 | 4 | 0 |
| Musculoskeletal | 13 | 2 | 0 |
| chest pain | | | |

Nervous system disorders

| | | | |
|---|---|---|---|
| Dizziness | 21 | 2 | 0 |
| Tremor | 19 | 3 | 0 |
| Headache | 17 | 0 | 0 |

Psychiatric disorders

| | | | |
|---|---|---|---|
| Insomnia | 23 | 2 | 0 |
| Anxiety | 13 | 0 | 0 |

Respiratory, thoracic and mediastinal disorders

| | | | |
|---|---|---|---|
| Cough^{e} | 43 | 1 | 0 |
| Dyspnea^{f} | 33 | 6 | 1 |
| Nasal congestion | 16 | 0 | 0 |

Key: D = Daratumumab, Pd = pomalidomide-dexamethasone.
^{a}Infusion reaction includes terms determined by investigators to be related to infusion, see section on Infusion Reactions below.
^{b}edema, edema peripheral, peripheral swelling.
^{c}acute tonsillitis, bronchitis, laryngitis, nasopharyngitis, pharyngitis, respiratory syncytial virus infection, rhinitis, sinusitis, tonsillitis, upper respiratory tract infection
^{d}lung infection, pneumonia, pneumonia aspiration
^{e}cough, productive cough, allergic cough
^{f}dyspnea, dyspnea exertional

Laboratory abnormalities worsening during treatment are listed in Table 21.

TABLE 21
Treatment-Emergent Hematology
Laboratory Abnormalities in EQUULEUS

| | DPd (N = 103) % | | |
|---|---|---|---|
| | Any Grade | Grade 3 | Grade 4 |
| Anemia | 57 | 30 | 0 |
| Thrombocytopenia | 75 | 10 | 10 |
| Neutropenia | 95 | 36 | 46 |
| Lymphopenia | 94 | 45 | 26 |

Key: D = Daratumumab, Pd = pomalidomide-dexamethasone.

### Monotherapy

The safety data reflect exposure to DARZALEX in 156 adult patients with relapsed and refractory multiple myeloma treated with DARZALEX at 16 mg/kg in three open-label, clinical trials. The median duration of exposure was 3.3 months (range: 0.03 to 20.04 months). Serious adverse reactions were reported in 51 (33%) patients. The most frequent serious adverse reactions were pneumonia (6%), general physical health deterioration (3%), and pyrexia (3%).

Adverse reactions resulted in treatment delay for 24 (15%) patients, most frequently for infections. Adverse reactions resulted in discontinuations for 6 (4%) patients.

Adverse reactions occurring in at least 10% of patients are presented in Table 22. Table 23 describes Grade 3-4 laboratory abnormalities reported at a rate of ≥10%.

TABLE 22
Adverse Reactions With Incidence ≥ 10% in Patients
With Multiple Myeloma Treated With DARZALEX 16 mg/kg

| | DARZALEX 16 mg/kg | | |
|---|---|---|---|
| | N = 156 | | |
| | Incidence (%) | | |
| Adverse Reaction | Any Grade | Grade 3 | Grade 4 |
| Infusion reaction^{a} | 48 | 3 | 0 |

General disorders and administration site conditions

| | | | |
|---|---|---|---|
| Fatigue | 39 | 2 | 0 |
| Pyrexia | 21 | 1 | 0 |
| Chills | 10 | 0 | 0 |

Respiratory, thoracic and mediastinal disorders

| | | | |
|---|---|---|---|
| Cough | 21 | 0 | 0 |
| Nasal congestion | 17 | 0 | 0 |
| Dyspnea | 15 | 1 | 0 |

Musculoskeletal and connective tissue disorders

| | | | |
|---|---|---|---|
| Back pain | 23 | 2 | 0 |
| Arthralgia | 17 | 0 | 0 |
| Pain in extremity | 15 | 1 | 0 |
| Musculoskeletal chest pain | 12 | 1 | 0 |

Infections and infestations

| | | | |
|---|---|---|---|
| Upper respiratory tract infection | 20 | 1 | 0 |
| Nasopharyngitis | 15 | 0 | 0 |
| Pneumonia^{b} | 11 | 6 | 0 |

Gastrointestinal disorders

| | | | |
|---|---|---|---|
| Nausea | 27 | 0 | 0 |
| Diarrhea | 16 | 1 | 0 |
| Constipation | 15 | 0 | 0 |
| Vomiting | 14 | 0 | 0 |

Metabolism and nutrition disorders

| | | | |
|---|---|---|---|
| Decreased appetite | 15 | 1 | 0 |

Nervous system disorders

| | | | |
|---|---|---|---|
| Headache | 12 | 1 | 0 |

Vascular disorders

| | | | |
|---|---|---|---|
| Hypertension | 10 | 5 | 0 |

^{a}Infusion reaction includes terms determined by investigators to be related to infusion, see section on Infusion Reactions below.
^{b}Pneumonia also includes the terms streptococcal pneumonia and lobar pneumonia.

TABLE 23
Treatment-Emergent Grade 3-4 Laboratory Abnormalities (≥10%)

| | Daratumumab 16 mg/kg (N = 156) | | |
|---|---|---|---|
| | Any Grade (%) | Grade 3 (%) | Grade 4 (%) |
| Anemia | 45 | 19 | 0 |
| Thrombocytopenia | 48 | 10 | 8 |
| Neutropenia | 60 | 17 | 3 |
| Lymphopenia | 72 | 30 | 10 |

### Infusion Reactions

In clinical trials (monotherapy and combination treatments; N=2,066) the incidence of any grade infusion reactions was 37% with the first (16 mg/kg, Week 1) infusion of DARZALEX, 2% with the Week 2 infusion, and cumulatively 6% with subsequent infusions. Less than 1% of patients had a Grade 3/4 infusion reaction at Week 2 or subsequent infusions.

The median time to onset of a reaction was 1.5 hours (range: 0 to 72.8 hours). The incidence of infusion modification due to reactions was 36%. Median durations of 16 mg/kg infusions for the 1^{st} week, 2^{nd} week and subsequent infusions were approximately 7, 4, and 3 hours respectively.

Severe infusion reactions included bronchospasm, dyspnea, laryngeal edema, pulmonary edema, hypoxia, and hypertension. Other adverse infusion reactions included nasal congestion, cough, chills, throat irritation, vomiting and nausea.

When DARZALEX dosing was interrupted in the setting of ASCT (Study CASSIOPEIA) for a median of 3.75 months (range: 2.4; 6.9 months), upon re-initiation of DARZALEX the incidence of IRRs was 11% for the first infusion following ASCT. Infusion rate/dilution volume used upon re-initiation was that used for the last DARZALEX infusion prior to interruption for ASCT. IRRs occurring at re-initiation of DARZALEX following ASCT were consistent in terms of symptoms and severity (Grade 3 or 4:<1%) with those reported in previous studies at Week 2 or subsequent infusions.

In EQUULEUS, patients receiving daratumumab combination treatment (n=97) were administered the first 16 mg/kg daratumumab dose at Week 1 split over two days i.e. 8 mg/kg on Day 1 and Day 2 respectively. The incidence of any grade infusion-related reactions was 42%, with 36% of patients experiencing infusion reactions on Day 1 of Week 1, 4% on Day 2 of Week 1, and 8% with subsequent infusions. The median time to onset of a reaction was 1.8 hours (range: 0.1 to 5.4 hours). The incidence of infusion interruptions due to reactions was 30%. Median durations of infusions were 4.2 h for Week 1-Day 1, 4.2 h for Week 1-Day 2, and 3.4 hours for the subsequent infusions.

### Herpes Zoster Virus Reactivation

Prophylaxis for Herpes Zoster Virus reactivation was recommended for patients in some clinical trials of DARZALEX. In monotherapy studies, herpes zoster was reported in 3% of patients. In the combination therapy studies, herpes zoster was reported in 2-5% of patients receiving DARZALEX.

### Infections

In patients receiving DARZALEX combination therapy, Grade 3 or 4 infections were reported as follows: Relapsed/refractory patient studies: DVd: 21%, Vd: 19%; DRd: 27%, Rd: 23%; DPd: 28% Newly diagnosed patient studies: D-VMP: 23%, VMP: 15%; DRd: 32%, Rd: 23%; DVTd: 22%; VTd 20%.

Pneumonia was the most commonly reported severe (Grade 3 or 4) infection across studies. In the active controlled studies, discontinuations from treatment due to infections (1-4%) and fatal infections were generally infrequent and balanced between the DARZALEX containing regimens and active control arms. Fatal infections were primarily due to pneumonia and sepsis.

### Hepatitis B Virus (HBV) Reactivation

Hepatitis B virus reactivation has been reported in less than 1% of patients (including fatal cases) treated with DARZALEX in clinical trials.

### 6.2 Immunogenicity

As with all therapeutic proteins, there is the potential for immunogenicity. The detection of antibody formation is highly dependent on the sensitivity and specificity of the assay. Additionally, the observed incidence of antibody (including neutralizing antibody) positivity in an assay may be influenced by several factors including assay methodology, sample handling, timing of sample collection, concomitant medications, and underlying disease. For these reasons, comparison of the incidence of antibodies to daratumumab in the studies described below with the incidence of antibodies in other studies or to other products may be misleading. In clinical trials of patients with multiple myeloma treated with DARZALEX as monotherapy or as combination therapies, none of the 111 evaluable monotherapy patients, and 2 of the 1,050 evaluable combination therapy patients, tested positive for anti-daratumumab antibodies. One patient administered DARZALEX as combination therapy, developed transient neutralizing antibodies against daratumumab. However, this assay has limitations in detecting anti-daratumumab antibodies in the presence of high concentrations of daratumumab; therefore, the incidence of antibody development might not have been reliably determined.

### 6.3 Postmarketing Experience

The following adverse reactions have been identified during post-approval use of DARZALEX. Because these reactions are reported voluntarily from a population of uncertain size, it is not always possible to reliably estimate their frequency or establish a causal relationship to drug exposure.

### Immune System Disorders: Anaphylactic Reaction

### 7 DRUG INTERACTIONS

### 7.1 Effects of Daratumumab on Laboratory Tests

Interference with Indirect Antiglobulin Tests (Indirect Coombs Test)

Daratumumab binds to CD38 on RBCs and interferes with compatibility testing, including antibody screening and cross matching. Daratumumab interference mitigation methods include treating reagent RBCs with dithiothreitol (DTT) to disrupt daratumumab binding [see References (15)] or genotyping. Since the Kell blood group system is also sensitive to DTT treatment, K-negative units should be supplied after ruling out or identifying alloantibodies using DTT-treated RBCs.

If an emergency transfusion is required, non-cross-matched ABO/RhD-compatible RBCs can be given per local blood bank practices.

Interference with Serum Protein Electrophoresis and Immunofixation Tests

Daratumumab may be detected on serum protein electrophoresis (SPE) and immunofixation (IFE) assays used for monitoring disease monoclonal immunoglobulins (M protein). False positive SPE and IFE assay results may occur for patients with IgG kappa myeloma protein impacting initial assessment of complete responses by International Myeloma Working Group (IMWG) criteria. In patients with persistent very good partial response, where daratumumab interference is suspected, consider using a FDA-approved daratumumab-specific IFE assay to distinguish daratumumab from any remaining endogenous M protein in the patient's serum, to facilitate determination of a complete response.

### 8 USE IN SPECIFIC POPULATIONS

### 8.1 Pregnancy

### Risk Summary

There are no human data to inform a risk with use of DARZALEX during pregnancy. Animal studies have not been conducted. However, there are clinical considerations (see Clinical Considerations). The estimated background risk of major birth defects and miscarriage for the indicated population is unknown. All pregnancies have a background risk of birth defect, loss, or other adverse outcomes. In the U.S. general population, the estimated background risk of major birth defects and miscarriage in clinically recognized pregnancies is 2-4% and 15-20%, respectively.

### Clinical Considerations

### Fetal/Neonatal Adverse Reactions

Immunoglobulin G1 (IgG1) monoclonal antibodies are transferred across the placenta. Based on its mechanism of action, DARZALEX may cause fetal myeloid or lymphoid-cell depletion and decreased bone density. Defer administering live vaccines to neonates and infants exposed to DARZALEX in utero until a hematology evaluation is completed.

### Data

### Animal Data

Mice that were genetically modified to eliminate all CD38 expression (CD38 knockout mice) had reduced bone density at birth that recovered by 5 months of age. In cynomolgus monkeys exposed during pregnancy to other monoclonal antibodies that affect leukocyte populations, infant monkeys had a reversible reduction in leukocytes.

### 8.2 Lactation

### Risk Summary

There is no information regarding the presence of daratumumab in human milk, the effects on the breastfed child, or the effects on milk production. Human IgG is known to be present in human milk. Published data suggest that antibodies in breast milk do not enter the neonatal and infant circulations in substantial amounts.

The developmental and health benefits of breast-feeding should be considered along with the mother's clinical need for DARZALEX and any potential adverse effects on the breast-fed child from DARZALEX or from the underlying maternal condition.

### 8.3 Females and Males of Reproductive Potential

### Contraception

To avoid exposure to the fetus, women of reproductive potential should use effective contraception during treatment and for 3 months after cessation of DARZALEX treatment.

### 8.4 Pediatric Use

Safety and effectiveness of DARZALEX in pediatric patients have not been established.

### 8.5 Geriatric Use

Of the 2,066 patients that received DARZALEX at the recommended dose, 37% were 65 to 75 years of age, and 16% were 75 years of age or older. No overall differences in safety or effectiveness were observed between these patients and younger patients [see Clinical Studies (14)].

### 11 DESCRIPTION

Daratumumab is an immunoglobulin G1 kappa (IgG1κ) human monoclonal antibody against CD38 antigen, produced in a mammalian cell line (Chinese Hamster Ovary [CHO]) using recombinant DNA technology. The molecular weight of daratumumab is approximately 148 kDa.

DARZALEX is supplied as a colorless to pale yellow preservative-free solution for intravenous infusion in single-dose vials. The pH is 5.5. DARZALEX must be diluted with 0.9% Sodium Chloride Injection, USP [see Dosage and Administration (2.4)].

Each DARZALEX single-dose 20 mL vial contains 400 mg daratumumab, glacial acetic acid (3.7 mg), mannitol (510 mg), polysorbate 20 (8 mg), sodium acetate trihydrate (59.3 mg), sodium chloride (70.1 mg), and water for injection.

Each DARZALEX single-dose 5 mL vial contains 100 mg daratumumab, glacial acetic acid (0.9 mg), mannitol (127.5 mg), polysorbate 20 (2 mg), sodium acetate trihydrate (14.8 mg), sodium chloride (17.5 mg), and water for injection

### 12 CLINICAL PHARMACOLOGY

### 12.1 Mechanism of Action

CD38 is a transmembrane glycoprotein (48 kDa) expressed on the surface of hematopoietic cells, including multiple myeloma and other cell types and tissues and has multiple functions, such as receptor mediated adhesion, signaling, and modulation of cyclase and hydrolase activity. Daratumumab is an IgG1κ human monoclonal antibody (mAb) that binds to CD38 and inhibits the growth of CD38 expressing tumor cells by inducing apoptosis directly through Fc mediated cross linking as well as by immune-mediated tumor cell lysis through complement dependent cytotoxicity (CDC), antibody dependent cell mediated cytotoxicity (ADCC) and antibody dependent cellular phagocytosis (ADCP). A subset of myeloid derived suppressor cells (CD38+MDSCs), regulatory T cells (CD38+T_{regs}) and B cells (CD38+B_{regs}) are decreased by daratumumab.

### 12.2 Pharmacodynamics

NK cells express CD38 and are susceptible to daratumumab mediated cell lysis. Decreases in absolute counts and percentages of total NK cells (CD16+CD56+) and activated (CD16+CD56^{dim}) NK cells in peripheral whole blood and bone marrow were observed with DARZALEX treatment.

### Cardiac Electrophysiology

DARZALEX as a large protein has a low likelihood of direct ion channel interactions. There is no evidence from non-clinical or clinical data to suggest that DARZALEX has the potential to delay ventricular repolarization.

### 12.3 Pharmacokinetics

Daratumumab area under the concentration-time curve (AUC) increases more than proportionally over a dosage range from 1 to 24 mg/kg (0.06 to 1.5 times the approved recommended dosage) as monotherapy or 1 to 16 mg/kg (0.06 to 1 time the approved recommended dosage) as combination therapy.

Following administration of the approved recommended dosage of DARZALEX as monotherapy or in combination therapy, the mean serum maximal concentration (Cₘₐₓ) was approximately 2.7 to 3-fold higher at the end of weekly dosing compared to the first dose. The mean±standard deviation (SD) trough serum concentration (Cₘᵢₙ) at the end of weekly dosing was 573±332 μg/mL when DARZALEX was administered as monotherapy and 502±196 to 607±231 μg/mL when DARZALEX was administered as combination therapy. Split dosing of the first dose resulted in a different PK profile in the first day compared to single dosing; however, similar Cₘₐₓ and Cₘᵢₙ concentrations were both predicted and observed following the administration of the second split dose on Week 1 Day 2.

When DARZALEX was administered as monotherapy, daratumumab steady state was achieved approximately 5 months into the every 4-week dosing period (by the 21^{st} infusion). At steady state, daratumumab mean±SD accumulation ratio for Cₘₐₓ was 1.6±0.5.

### Distribution

Daratumumab volume of distribution was 4.7±1.3 L as monotherapy and 4.4±1.5 L as combination therapy following administration of the approved dosage.

### Elimination

Daratumumab clearance decreased with increasing dose and with multiple dosing. The mean±SD linear clearance was estimated to be 171.4±95.3 mL/day and the mean±SD estimated terminal half-life associated with linear clearance was 18±9 days following administration of the approved recommended dosage of DARZALEX as monotherapy. Terminal half-life was similar when DARZALEX was administered as combination therapy.

### Specific Populations

No clinically significant differences in the pharmacokinetics of daratumumab as monotherapy or as combination therapy were observed based on sex, age (31 to 93 years), mild [total bilirubin 1 to 1.5 times upper limit of normal (ULN) or aspartate aminotransaminase (AST)>ULN] and moderate (total bilirubin 1.5 to 3 times ULN and any AST) hepatic impairment, or renal impairment [Creatinine clearance (CLcr) 15-89 mL/min]. The effect of severe (total bilirubin >3 times ULN and any AST) hepatic impairment on daratumumab pharmacokinetics is unknown.

### Body Weight

The central volume of distribution and clearance of daratumumab increased with increasing body weight.

### 13 NONCLINICAL TOXICOLOGY

### 13.1 Carcinogenesis, Mutagenesis, Impairment of Fertility

No carcinogenicity or genotoxicity studies have been conducted with daratumumab. No animal studies have been performed to evaluate the potential effects of daratumumab on reproduction or development, or to determine potential effects on fertility in males or females.

### 14 CLINICAL STUDIES

### 14.1 Newly Diagnosed Multiple Myeloma

Combination Treatment with Lenalidomide and Dexamethasone in Patients Ineligible for Autologous Stem Cell Transplant

MAIA (NCT02252172), an open-label, randomized, active-controlled Phase 3 study, compared treatment with DARZALEX 16 mg/kg in combination with lenalidomide and low-dose dexamethasone (DRd) to treatment with lenalidomide and low-dose dexamethasone (Rd) in patients with newly diagnosed multiple myeloma ineligible for autologous stem cell transplant. Lenalidomide (25 mg once daily orally on Days 1-21 of repeated 28-day [4-week] cycles) was given with low dose oral or intravenous dexamethasone 40 mg/week (or a reduced dose of 20 mg/week for patients >75 years or body mass index [BMI]<18.5). On DARZALEX infusion days, the dexamethasone dose was given as a pre-infusion medication. Dose adjustments for lenalidomide and dexamethasone were applied according to manufacturer's prescribing information. Treatment was continued in both arms until disease progression or unacceptable toxicity.

A total of 737 patients were randomized: 368 to the DRd arm and 369 to the Rd arm. The baseline demographic and disease characteristics were similar between the two treatment groups. The median age was 73 (range: 45-90) years, with 44% of the patients ≥75 years of age. The majority were white (92%), male (52%), 34% had an Eastern Cooperative Oncology Group (ECOG) performance score of 0, 50% had an ECOG performance score of 1 and 17% had an ECOG performance score of ≥2. Twenty-seven percent had International Staging System (ISS) Stage I, 43% had ISS Stage II and 29% had ISS Stage III disease. Efficacy was evaluated by progression free survival (PFS) based on International Myeloma Working Group (IMWG) criteria.

MAIA demonstrated an improvement in Progression Free Survival (PFS) in the DRd arm as compared to the Rd arm; the median PFS had not been reached in the DRd arm and was 31.9 months in the Rd arm (hazard ratio [HR]=0.56; 95% CI: 0.43, 0.73; p<0.0001), representing 44% reduction in the risk of disease progression or death in patients treated with DRd.

Additional efficacy results from MAIA are presented in Table 24 below.

TABLE 24
Additional Efficacy Results From MAIA^{a}

| | DRd (N = 368) | | Rd (N = 369) | |
|---|---|---|---|---|
| Overall response (sCR + | 342 | (92.9%) | 300 | (81.3%) |
| CR + VGPR + PR) n(%)^{a} | | | | |
| p-value^{b} | <0.0001 | | | |
| Stringent complete | 112 | (30.4%) | 46 | (12.5%) |
| response (sCR) | | | | |
| Complete response (CR) | 63 | (17.1%) | 46 | (12.5%) |
| Very good partial | 117 | (31.8%) | 104 | (28.2%) |
| response (VGPR) | | | | |
| Partial response (PR) | 50 | (13.6%) | 104 | (28.2%) |
| CR or better (sCR + CR) | 175 | (47.6%) | 92 | (24.9%) |
| p-value^{b} | <0.0001 | | | |
| VGPR or better | 292 | (79.3%) | 196 | (53.1%) |
| (sCR + CR + VGPR) | | | | |
| p-value^{b} | <0.0001 | | | |
| MRD negativity rate^{a, c} n(%) | 89 | (24.2%) | 27 | (7.3%) |
| 95% CI (%) | (19.9%, 28.9%) | | (4.9%, 10.5%) | |
| p-value^{d} | <0.0001 | | | |
| MRD negativity rate in | | | | |
| patients with CR or better^{c} | | | | |
| Number of patients | N = 175 | | N = 92 | |
| with CR or better | | | | |
| MRD negativity rate n(%) | 89 | (50.9%) | 27 | (29.3%) |

| | | |
|---|---|---|
| 95% CI (%) | (43.2%, 58.5%) | (20.3%, 39.8%) |

DRd = daratumumab-lenalidomide-dexamethasone; Rd = lenalidomide-dexamethasone; MRD = minimal residual disease; CI = confidence interval
^{a}Based on intent-to-treat population
^{b}p-value from Cochran Mantel-Haenszel Chi-Squared test.
^{c}Based on threshold of 10⁻⁵ using a next-generation sequencing assay (ClonoSEQ).
^{d}p-value from Fisher's exact test

In responders, the median time to response was 1.05 months (range: 0.2 to 12.1 months) in the DRd group and 1.05 months (range: 0.3 to 15.3 months) in the Rd group. The median duration of response had not been reached in the DRd group and was 34.7 months (95% CI: 30.8, not estimable) in the Rd group.

Combination Treatment with Bortezomib, Melphalan and Prednisone (VMP) in Patients Ineligible for Autologous Stem Cell Transplant

ALCYONE (NCT02195479), an open-label, randomized, active-controlled Phase 3 study, compared treatment with DARZALEX 16 mg/kg in combination with bortezomib, melphalan and prednisone (D-VMP), to treatment with VMP in patients with newly diagnosed multiple myeloma ineligible for autologous stem cell transplant. Bortezomib was administered by subcutaneous (SC) injection at a dose of 1.3 mg/m² body surface area twice weekly at Weeks 1, 2, 4 and 5 for the first 6-week cycle (Cycle 1; 8 doses), followed by once weekly administrations at Weeks 1, 2, 4 and 5 for eight more 6-week cycles (Cycles 2-9; 4 doses per cycle). Melphalan at 9 mg/m², and prednisone at 60 mg/m² were orally administered on Days 1 to 4 of the nine 6-week cycles (Cycles 1-9). DARZALEX treatment was continued until disease progression or unacceptable toxicity.

A total of 706 patients were randomized: 350 to the D-VMP arm and 356 to the VMP arm. The baseline demographic and disease characteristics were similar between the two treatment groups. The median age was 71 (range: 40-93) years, with 30% of the patients ≥75 years of age. The majority were white (85%), female (54%), 25% had an ECOG performance score of 0, 50% had an ECOG performance score of 1 and 25% had an ECOG performance score of 2. Nineteen percent of patients had ISS Stage I, 42% had ISS Stage II and 38% had ISS Stage III disease. Efficacy was evaluated by PFS based on IMWG criteria.

ALCYONE demonstrated an improvement in PFS in the D-VMP arm as compared to the VMP arm; the median PFS had not been reached in the D-VMP arm and was 18.1 months (95% CI:16.53, 19.91) in the VMP arm (HR=0.5; 95% CI: 0.38, 0.65; p<0.0001), representing 50% reduction in the risk of disease progression or death in patients treated with D-VMP.

Additional efficacy results from ALCYONE are presented in Table 25 below.

TABLE 25
Additional Efficacy Results From ALCYONE

| | D-VMP (N = 350) | | VMP (N = 356) | |
|---|---|---|---|---|
| Overall response (sCR + | 318 | (90.9%) | 263 | (73.9%) |
| CR + VGPR + PR) n(%)^{a} | | | | |
| p-value^{b} | <0.0001 | | | |
| Stringent complete | 63 | (18.0%) | 25 | (7.0%) |
| response (sCR) | | | | |
| Complete response (CR) | 86 | (24.6%) | 62 | (17.4%) |
| Very good partial | 100 | (28.6%) | 90 | (25.3%) |
| response (VGPR) | | | | |
| Partial response (PR) | 69 | (19.7%) | 86 | (24.2%) |
| MRD negativity rate^{a, c} n(%) | 78 | (22.3%) | 22 | (6.2%) |
| 95% CI (%) | (18.0, 27.0) | | (3.9, 9.2) | |
| p-value^{d} | <0.0001 | | | |
| MRD negativity rate in | | | | |
| patients with CR or better^{c} | | | | |
| Number of patients with | N = 149 | | N = 87 | |
| CR or better | | | | |
| MRD negativity rate n(%) | 74 | (49.7%) | 22 | (25.3%) |

| | | |
|---|---|---|
| 95% CI (%) | (41.4, 58.0) | (16.6, 35.7) |

D-VMP = daratumumab-bortezomib-melphalan-prednisone; VMP = bortezomib-melphalan-prednisone; MRD = minimal residual disease; CI = confidence interval
aBased on intent-to-treat population
^{b}p-value from Cochran Mantel-Haenszel Chi-Squared test.
^{c}Based on threshold of 10⁻⁵ using a next-generation sequencing assay (ClonoSEQ).
^{d}p-value from Fisher's exact test.

In responders, the median time to response was 0.79 months (range: 0.4 to 15.5 months) in the D-VMP group and 0.82 months (range: 0.7 to 12.6 months) in the VMP group. The median duration of response had not been reached in the D-VMP group and was 21.3 months (range: 0.5+, 23.7+) in the VMP group.

Combination Treatment with Bortezomib, Thalidomide and Dexamethasone in Patients Eligible for Autologous Stem Cell Transplant (ASCT)

CASSIOPEIA (NCT02541383), an open-label, randomized, active-controlled Phase 3 study compared induction and consolidation treatment with DARZALEX 16 mg/kg in combination with bortezomib, thalidomide and dexamethasone (DVTd) to treatment with bortezomib, thalidomide and dexamethasone (VTd) in patients with newly diagnosed multiple myeloma eligible for ASCT. The consolidation phase of treatment began a minimum of 30 days post-ASCT, when the patient had recovered sufficiently, and engraftment was complete. The trial was limited to patients 65 years of age and younger.

Bortezomib was administered by subcutaneous (SC) injection or intravenous (IV) injection at a dose of 1.3 mg/m² body surface area twice weekly for two weeks (Days 1, 4, 8, and 11) of repeated 28-day (4-week) induction treatment cycles (Cycles 1-4) and two consolidation cycles (Cycles 5 and 6) following ASCT after Cycle 4. Thalidomide was administered orally at 100 mg daily during the six bortezomib cycles. Dexamethasone (oral or intravenous) was administered at 40 mg on Days 1, 2, 8, 9, 15, 16, 22 and 23 of Cycles 1 and 2, and at 40 mg on Days 1-2 and 20 mg on subsequent dosing days (Days 8, 9, 15, 16) of Cycles 3-4. Dexamethasone 20 mg was administered on Days 1, 2, 8, 9, 15, 16 in Cycles 5 and 6. On the days of DARZALEX infusion, the dexamethasone dose was administered intravenously as a pre-infusion medication. Dose adjustments for bortezomib, thalidomide and dexamethasone were applied according to manufacturer's prescribing information.

A total of 1,085 patients were randomized: 543 to the DVTd arm and 542 to the VTd arm. The baseline demographic and disease characteristics were similar between the two treatment groups. The median age was 58 years (range: 22 to 65 years). The majority were male (59%), 48% had an ECOG performance score of 0, 42% had an ECOG performance score of 1 and 10% had an ECOG performance score of 2. Forty percent had ISS Stage I, 45% had ISS Stage II and 15% had ISS Stage III disease.

Efficacy was evaluated by stringent Complete Response (sCR) rate at Day 100 post-transplant, Complete Response Rate (CR) at Day 100 post-transplant, and Progression-Free Survival (PFS).

TABLE 26
Efficacy Results From CASSIOPEIA at Day 100 Post-Transplant

| | DVTd (N = 543) | | VTd (N = 542) | |
|---|---|---|---|---|
| Overall response (sCR + CR + | 503 | (92.6%) | 487 | (89.9%) |
| VGPR + PR) n(%)^{a} | | | | |
| Stringent complete response (sCR) | 157 | (28.9%) | 110 | (20.3%) |
| p-value^{b} | 0.0010 | | | |
| Complete response (CR) | 54 | (9.9%) | 31 | (5.7%) |
| Very good partial response (VGPR) | 242 | (44.6%) | 282 | (52.0%) |
| Partial response (PR) | 50 | (9.2%) | 64 | (11.8%) |

D-VTd = daratumumab-bortezomib-thalidomide-dexamethasone. VTd = bortezomib-thalidomide-dexamethasone
^{a}Based on intent-to-treat population
^{b}p-value from Cochran Mantel-Haenszel Chi-Squared test.

CASSIOPEIA demonstrated an improvement in PFS in the DVTd arm as compared to the VTd arm; with a median follow up of 18.8 months, the median PFS had not been reached in either arm. Treatment with DVTd resulted in a reduction in the risk of progression or death by 53% compared to VTd alone (HR=0.47; 95% CI: 0.33, 0.67; p<0.0001).

### 14.2 Relapsed/Refractory Multiple Myeloma

Combination Treatment with Lenalidomide and Dexamethasone

POLLUX (NCT02076009), an open-label, randomized, active-controlled Phase 3 trial, compared treatment with DARZALEX 16 mg/kg in combination with lenalidomide and low-dose dexamethasone (DRd) to treatment with lenalidomide and low-dose dexamethasone (Rd) in patients with multiple myeloma who had received at least one prior therapy. Lenalidomide (25 mg once daily orally on Days 1-21 of repeated 28-day [4-week] cycles) was given with low dose oral or intravenous dexamethasone 40 mg/week (or a reduced dose of 20 mg/week for patients >75 years or BMI <18.5). On DARZALEX infusion days, 20 mg of the dexamethasone dose was given as a pre-infusion medication and the remainder given the day after the infusion. For patients on a reduced dexamethasone dose, the entire 20 mg dose was given as a DARZALEX pre-infusion medication. Dose adjustments for lenalidomide and dexamethasone were applied according to manufacturer's prescribing information. Treatment was continued in both arms until disease progression or unacceptable toxicity.

A total of 569 patients were randomized; 286 to the DRd arm and 283 to the Rd arm. The baseline demographic and disease characteristics were similar between the DARZALEX and the control arm. The median patient age was 65 years (range 34 to 89 years), 11% were ≥75 years, 59% were male; 69% Caucasian, 18% Asian, and 3% African American. Patients had received a median of 1 prior line of therapy. Sixty-three percent (63%) of patients had received prior autologous stem cell transplantation (ASCT). The majority of patients (86%) received a prior PI, 55% of patients had received a prior immunomodulatory agent, including 18% of patients who had received prior lenalidomide; and 44% of patients had received both a prior PI and immunomodulatory agent. At baseline, 27% of patients were refractory to the last line of treatment. Eighteen percent (18%) of patients were refractory to a PI only, and 21% were refractory to bortezomib. Efficacy was evaluated by PFS based on IMWG criteria.

POLLUX demonstrated an improvement in PFS in the DRd arm as compared to the Rd arm; the median PFS had not been reached in the DRd arm and was 18.4 months in the Rd arm (HR=0.37; 95% CI: 0.27, 0.52; p<0.0001), representing 63% reduction in the risk of disease progression or death in patients treated with DRd.

Additional efficacy results from POLLUX are presented in Table 27 below.

TABLE 27
Additional Efficacy Results From POLLUX^{a}

| | DRd (N = 286) | | Rd (N = 283) | |
|---|---|---|---|---|
| Overall response (sCR + | 261 | (91.3%) | 211 | (74.6%) |
| CR + VGPR + PR) | | | | |
| p-value^{b} | <0.0001 | | | |
| Stringent complete | 51 | (17.8%) | 20 | (7.1%) |
| response (sCR) | | | | |
| Complete response (CR) | 70 | (24.5%) | 33 | (11.7%) |
| Very good partial response | 92 | (32.2%) | 69 | (24.4%) |
| (VGPR) | | | | |
| Partial response (PR) | 48 | (16.8%) | 89 | (31.4%) |

DRd = daratumumab- lenalidomide-dexamethasone; Rd = lenalidomide-dexamethasone
^{a}Based on Intent-to-treat population
^{b}p-value from Cochran Mantel-Haenszel Chi-Squared test.

In responders, the median time to response was 1 month (range: 0.9 to 13 months) in the DRd group and 1.1 months (range: 0.9 to 10 months) in the Rd group. The median duration of response had not been reached in the DRd group (range: 1+ to 19.8+ months) and was 17.4 months (range: 1.4 to 18.5+ months) in the Rd group.

With a median follow-up of 13.5 months, 75 deaths were observed; 30 in the DRd group and 45 in the Rd group.

Combination Treatment with Bortezomib and Dexamethasone

CASTOR (NCT02136134), an open-label, randomized, active-controlled Phase 3 trial, compared treatment with DARZALEX 16 mg/kg in combination with bortezomib and dexamethasone (DVd), to treatment with bortezomib and dexamethasone (Vd) in patients with multiple myeloma who had received at least one prior therapy. Bortezomib was administered by SC injection or IV infusion at a dose of 1.3 mg/m² body surface area twice weekly for two weeks (Days 1, 4, 8, and 11) of repeated 21 day (3-week) treatment cycles, for a total of 8 cycles. Dexamethasone was administered orally at a dose of 20 mg on Days 1, 2, 4, 5, 8, 9, 11, and 12 of each of the 8 bortezomib cycles (80 mg/week for two out of three weeks of the bortezomib cycle) or a reduced dose of 20 mg/week for patients >75 years, BMI <18.5, poorly controlled diabetes mellitus or prior intolerance to steroid therapy. On the days of DARZALEX infusion, 20 mg of the dexamethasone dose was administered as a pre-infusion medication. For patients on a reduced dexamethasone dose, the entire 20 mg dose was given as a DARZALEX pre-infusion medication. Bortezomib and dexamethasone were given for 8 three-week cycles in both treatment arms; whereas DARZALEX was given until disease progression. However, dexamethasone 20 mg was continued as a DARZALEX pre-infusion medication in the DVd arm. Dose adjustments for bortezomib and dexamethasone were applied according to manufacturer's prescribing information.

A total of 498 patients were randomized; 251 to the DVd arm and 247 to the Vd arm. The baseline demographic and disease characteristics were similar between the DARZALEX and the control arm. The median patient age was 64 years (range 30 to 88 years); 12% were ≥75 years, 57% were male; 87% Caucasian, 5% Asian and 4% African American. Patients had received a median of 2 prior lines of therapy and 61% of patients had received prior autologous stem cell transplantation (ASCT). Sixty-nine percent (69%) of patients had received a prior PI (66% received bortezomib) and 76% of patients received an immunomodulatory agent (42% received lenalidomide). At baseline, 32% of patients were refractory to the last line of treatment and the proportions of patients refractory to any specific prior therapy were in general well balanced between the treatment groups. Thirty-three percent (33%) of patients were refractory to an immunomodulatory agent only, with 24% patients in the DVd arm and 33% of patients in the Vd arm respectively refractory to lenalidomide. Efficacy was evaluated by PFS based on IMWG criteria.

CASTOR demonstrated an improvement in PFS in the DVd arm as compared to the Vd arm; the median PFS had not been reached in the DVd arm and was 7.2 months in the Vd arm (HR [95% CI]: 0.39 [0.28, 0.53]; p-value <0.0001), representing a 61% reduction in the risk of disease progression or death for patients treated with DVd versus Vd.

Additional efficacy results from CASTOR are presented in Table 28 below.

TABLE 28
Additional Efficacy Results From CASTOR^{a}

| | DVd (N = 251) | | Vd (N = 247) | |
|---|---|---|---|---|
| Overall response (sCR + | 199 | (79.3%) | 148 | (59.9%) |
| CR + VGPR + PR) | | | | |
| P-value^{b} | <.0001 | | | |
| Stringent complete | 11 | (4.4%) | 5 | (2.0%) |
| response (sCR) | | | | |
| Complete response (CR) | 35 | (13.9%) | 16 | (6.5%) |
| Very good partial | 96 | (38.2%) | 47 | (19.0%) |
| response (VGPR) | | | | |
| Partial response (PR) | 57 | (22.7%) | 80 | (32.4%) |

DVd = daratumumab-bortezomib-dexamethasone; Vd = bortezomib-dexamethasone
^{a}Based on Intent-to-treat population
^{b}p-value from Cochran Mantel-Haenszel Chi-Squared test.

In responders, the median time to response was 0.8 months (range: 0.7 to 4 months) in the DVd group and 1.5 months (range: 0.7 to 5 months) in the Vd group. The median duration of response had not been reached in the DVd group (range: 1.4+ to 14.1+ months) and was 7.9 months (1.4+ to 12+ months) in the Vd group.

With a median follow-up of 7.4 months, 65 deaths were observed; 29 in the DVd group and 36 in the Vd group were observed.

Combination Treatment with Pomalidomide and Dexamethasone

EQUULEUS (NCT01998971) was an open-label trial in which 103 patients with multiple myeloma who had received a prior PI and an immunomodulatory agent, received 16 mg/kg DARZALEX in combination with pomalidomide and low-dose dexamethasone until disease progression. Pomalidomide (4 mg once daily orally on Days 1-21 of repeated 28-day [4-week] cycles) was given with low dose oral or intravenous dexamethasone 40 mg/week (reduced dose of 20 mg/week for patients >75 years or BMI <18.5). On DARZALEX infusion days, 20 mg of the dexamethasone dose was given as a pre-infusion medication and the remainder given the day after the infusion. For patients on a reduced dexamethasone dose, the entire 20 mg dose was given as a DARZALEX pre-infusion medication.

The median patient age was 64 years (range: 35 to 86 years) with 8% of patients ≥75 years of age. Patients in the study had received a median of 4 prior lines of therapy. Seventy-four percent (74%) of patients had received prior ASCT. Ninety-eight percent (98%) of patients received prior bortezomib treatment, and 33% of patients received prior carfilzomib. All patients received prior lenalidomide treatment, with 98% of patients previously treated with the combination of bortezomib and lenalidomide. Eighty nine percent (89%) of patients were refractory to lenalidomide and 71% refractory to bortezomib; 64% of patients were refractory to bortezomib and lenalidomide.

Efficacy results were based on overall response rate as determined by Independent Review Committee using IMWG criteria (see Table 29).

TABLE 29
Efficacy Results for EQUULEUS

| | N = 103 | |
|---|---|---|
| Overall response rate (ORR) | 61 | (59.2%) |
| 95% CI (%) | (49.1, 68.8) | |
| Stringent complete response (sCR) | 8 | (7.8%) |
| Complete response (CR) | 6 | (5.8%) |
| Very good partial response (VGPR) | 29 | (28.2%) |
| Partial response (PR) | 18 | (17.5%) |

ORR = sCR + CR + VGPR + PR
CI = Confidence Interval

The median time to response was 1 month (range: 0.9 to 2.8 months). The median duration of response was 13.6 months (range: 0.9+ to 14.6+ months).

### Monotherapy

SIRIUS (NCT01985126), was an open-label trial evaluating DARZALEX monotherapy in patients with relapsed or refractory multiple myeloma who had received at least 3 prior lines of therapy including a proteasome inhibitor and an immunomodulatory agent or who were double-refractory to a proteasome inhibitor and an immunomodulatory agent. In 106 patients, DARZALEX 16 mg/kg was administered with pre- and post-infusion medication. Treatment continued until unacceptable toxicity or disease progression.

The median patient age was 63.5 years (range: 31 to 84 years), 49% were male and 79% were Caucasian. Patients had received a median of 5 prior lines of therapy. Eighty percent of patients had received prior autologous stem cell transplantation (ASCT). Prior therapies included bortezomib (99%), lenalidomide (99%), pomalidomide (63%) and carfilzomib (50%). At baseline, 97% of patients were refractory to the last line of treatment, 95% were refractory to both, a proteasome inhibitor (PI) and immunomodulatory agent, and 77% were refractory to alkylating agents.

Efficacy results were based on overall response rate as determined by the Independent Review Committee assessment using IMWG criteria (see Table 30).

TABLE 30
Efficacy Results for SIRIUS

| | N = 106 | |
|---|---|---|
| Overall response rate (ORR) | 31 | (29.2%) |
| 95% CI (%) | (20.8, 38.9) | |
| Stringent complete response (sCR) | 3 | (2.8%) |
| Complete response (CR) | 0 | |
| Very good partial response (VGPR) | 10 | (9.4%) |
| Partial response (PR) | 18 | (17.0%) |

ORR = sCR + CR + VGPR + PR
CI = confidence interval

The median time to response was 1 month (range: 0.9 to 5.6 months). The median duration of response was 7.4 months (range: 1.2 to 13.1+ months).

Study GEN501 (NCT00574288) was an open-label dose escalation trial evaluating DARZALEX monotherapy in patients with relapsed or refractory multiple myeloma who had received at least 2 different cytoreductive therapies. In 42 patients, DARZALEX 16 mg/kg was administered with pre- and post-infusion medication. Treatment continued until unacceptable toxicity or disease progression.

The median patient age was 64 years (range: 44 to 76 years), 64% were male and 76% were Caucasian. Patients in the study had received a median of 4 prior lines of therapy. Seventy-four percent of patients had received prior ASCT. Prior therapies included bortezomib (100%), lenalidomide (95%), pomalidomide (36%) and carfilzomib (19%). At baseline, 76% of patients were refractory to the last line of treatment, 64% of patients were refractory to both, a PI and an immunomodulatory agent, and 60% of patients were refractory to alkylating agents.

Overall response rate was 36% (95% CI: 21.6, 52.0%) with 1 CR and 3 VGPR. The median time to response was 1 month (range: 0.5 to 3.2 months). The median duration of response was not estimable (range: 2.2 to 13.1+ months).

### 15 REFERENCES

1. Chapuy, C I, R T Nicholson, M D Aguad, et al., 2015, Resolving the daratumumab interference with blood compatibility testing, Transfusion, 55:1545-1554 (accessible at http://onlinelibrary.wiley.com/doi/10.1111/trf.13069/epdf).

### 16 HOW SUPPLIED/STORAGE AND HANDLING

### 16.1 How Supplied

DARZALEX is a colorless to pale yellow, preservative-free solution for intravenous infusion supplied as:

NDC 57894-502-05 contains one 100 mg/5 mL single-dose vial
NDC 57894-502-20 contains one 400 mg/20 mL single-dose vial

### 16.2 Storage and Stability

Store in a refrigerator at 2° C. to 8° C. (36° F. to 46° F.).

Do not freeze or shake. Protect from light. This product contains no preservative.

### 17 PATIENT COUNSELING INFORMATION

Advise the patient to read the FDA-approved patient labeling (Patient Information).

### Infusion Reactions

Advise patients to seek immediate medical attention for any of the following signs and symptoms of infusion reactions:
itchy, runny or blocked nose; chills, nausea, throat irritation, cough, headache, shortness of breath or difficulty breathing [see Warnings and Precautions (5.1) and Adverse Reactions (6.1)].

### Neutropenia

Advise patients that if they have a fever, they should contact their healthcare professional [see Warnings and Precautions (5.3) and Adverse Reactions (6.1)].

### Thrombocytopenia

Advise patients to inform their healthcare professional if they notice signs of bruising or bleeding [see Warnings and Precautions (5.4) and Adverse Reactions (6.1)].
   Interference with Laboratory Tests

Advise patients to inform healthcare providers including blood transfusion centers/personnel that they are taking DARZALEX, in the event of a planned transfusion [see Warnings and Precautions (5.2) and Drug Interactions (7.1)].

Advise patients that DARZALEX can affect the results of some tests used to determine complete response in some patients and additional tests may be needed to evaluate response [see Warnings and Precautions (5.5) and Drug Interactions (7.1)].

### Hepatitis B Virus (HBV) Reactivation:

Advise patients to inform healthcare providers if they have ever had or might have a hepatitis B infection and that DARZALEX could cause hepatitis B virus to become active again [see Adverse Reactions (6.1)].

### Manufactured by:

### Janssen Biotech, Inc.

### Horsham, Pa. 19044

### U.S. License Number 1864

What is claimed is:

1) A method of treating a subject with newly diagnosed multiple myeloma, comprising:
a) providing a healthcare professional (HCP) daratumumab;
b) providing the HCP information that a combination therapy comprising daratumumab, bortezomib, thalidomide and dexamethasone (DVTd) is demonstrated to increase a likelihood of achieving a stringent complete response (sCR) or better in subjects with newly diagnosed multiple myeloma; wherein performing the steps a) and b) results in the subject with newly diagnosed multiple myeloma to receive combination therapy comprising DVTd by the HCP or by self-administration as instructed by the HCP, thereby treating the subject having the newly diagnosed multiple myeloma.

2) The method of claim 1, wherein the subject is eligible for autologous stem cell transplant (ASCT).

3) The method of claim 1, wherein the likelihood of achieving the sCR or better is about 28% or higher.

4) The method of claim 3, wherein the likelihood of achieving the sCR or better is about 38% or higher.

5) The method of claim 1, wherein the combination therapy comprising DVTd is demonstrated to increase a likelihood of achieving a negative status for minimal residual disease (MRD) in subjects with newly diagnosed multiple myeloma.

6) The method of claim 5, wherein the likelihood of achieving the negative status for MRD is about 33% or higher.

7) The method of claim 1, wherein the combination therapy comprising DVTd is demonstrated to reduce a risk of progression of multiple myeloma or death in subjects with newly diagnosed multiple myeloma.

8) The method of claim 7, wherein the risk of progression of multiple myeloma or death is reduced by about 53%.

9) The method of claim 1, wherein the combination therapy comprises about 16 mg/kg daratumumab, about 1.3 mg/m² bortezomib, about 100 mg thalidomide and between about 20 mg and about 40 mg dexamethasone.

10) The method of claim 1, wherein the combination therapy comprises an induction phase, a high dose chemotherapy (HDC) and autologous stem cell transplant (ASCT), and a consolidation phase.

11) The method of claim 10, wherein the induction phase comprises four 28-day induction cycles comprising
a. about 16 mg/kg daratumumab administered once a week on weeks 1 to 8 and once in two weeks on weeks 9-16;
b. about 1.3 mg/m² bortezomib administered twice a week on week 1 and week 2 in the four 28-day induction cycles;
c. about 100 mg thalidomide daily; and
d. about 40 mg dexamethasone administered twice a week on week 1, week 2 and week 3 in the first and the second 28-day induction cycle, about 40 mg twice a week on week 1 and about 20 mg twice a week on week 2 and 3 in the third and the fourth 28-day induction cycle.

12) The method of claim 11, wherein the induction phase comprises four 28-day induction cycles comprising
a) about 16 mg/kg daratumumab administered once a week on weeks 1 to 8 and once in two weeks on weeks 9-16;
b) about 1.3 mg/m² bortezomib administered on days 1, 4, 8 and 11 in the four 28-day induction cycle;
c) about 100 mg thalidomide daily; and
d) about 40 mg dexamethasone administered on days 1, 2, 8, 9, 15, 16 in the first and the second 28-day induction cycle, about 40 mg on days 1 and 2 and about 20 mg on days 8, 9, 15 and 16 in the third and the fourth 28-day induction cycle.

13) The method of claim 11, wherein the induction phase is followed by the HDC and ASCT.

14) The method of claim 13, wherein the HDC comprises melphalan.

15) The method of claim 14, wherein melphalan is administered at a dose of about 200 mg/m², optionally over a period of 24 to 48 hours.

16) The method of claim 13, wherein the HDC and ASCT is followed by the consolidation phase.

17) The method of claim 10, wherein the consolidation phase comprises two 28-day consolidation cycles comprising
e. about 16 mg/kg daratumumab administered once in two weeks on weeks 1 to 8;
f. about 1.3 mg/m² bortezomib administered twice a week on week 1 and week 2 in each two 28-day consolidation cycle;
g. about 100 mg thalidomide daily; and
h. about 20 mg dexamethasone administered twice a week on week 1, week 2 and week 3 in each two 28-day consolidation cycle.

18) The method of claim 17, wherein the consolidation phase comprises two 28-day consolidation cycles of
a) about 16 mg/kg daratumumab on days 1 and 15 in each two 28-day consolidation cycle;
b) about 1.3 mg/m² bortezomib on days 1, 4, 8 and 11 in each two 28-day consolidation cycles;
c) about 100 mg thalidomide daily; and
d) about 20 mg dexamethasone on days 1, 2, 8, 9, 15 and 16 in each two 28-day consolidation cycles.

19) The method of claim 1, wherein the combination therapy comprises administering dexamethasone as pre-medication on daratumumab administration days.

20) The method of claim 1, wherein the combination therapy comprises administering daratumumab intravenously, bortezomib subcutaneously or intravenously, thalidomide orally and dexamethasone intravenously or orally.

21) The method of claim 20, wherein thalidomide, dexamethasone or both thalidomide and dexamethasone are self-administered.

22) The method of claim 1, wherein the information that a combination therapy comprising DVTd is demonstrated to increase a likelihood of achieving a stringent complete response (sCR) or better in subjects with newly diagnosed multiple myeloma is provided in a daratumumab-containing drug product label.

23) The method of claim 22, wherein the daratumumab-containing drug product label includes information that a recommended dose of daratumumab is 16 mg/kg administered as an intravenous injection.

24) The method of claim 23, wherein the daratumumab-containing drug product label includes information that the recommended dosing schedule of daratumumab in combination with bortezomib, thalidomide and dexamethasone is once a week on weeks 1 to 8 and once in two weeks on weeks 9-24 during the induction phase and once every two weeks on weeks 1 to 8 during the consolidation phase.

25) The method of claim 24, wherein the daratumumab-containing drug product label includes information that the recommended dosing schedule of bortezomib is 1.3 mg/m² bortezomib on days 1, 4, 8 and 11 in the four 28-day induction cycles and on days 1, 4, 8 and 11 in the two 28-day consolidation cycles.

26) The method of claim 25, wherein the daratumumab-containing drug product label includes information that the recommended dosing schedule of thalidomide is 100 mg daily.

27) The method of claim 26, wherein the daratumumab-containing drug product label includes information that the recommended dosing schedule of dexamethasone is about 40 mg on days 1, 2, 8, 9, 15, 16 in the first and the second 28-day induction cycle, about 40 mg on days 1-2 and about 20 mg on days 8, 9, 15 and 16 in the third and the fourth 28-day induction cycle, and about 20 mg on days 1, 2, 8, 9, 15, 16 in the first and the second 28-day consolidation cycle.

28) The method of claim 22, wherein daratumumab, bortezomib, thalidomide and dexamethasone are administered according to the recommended dosing schedules.

29) The method of claim 22, wherein the daratumumab-containing drug product label includes data from an open-label, randomized active-controlled phase 3 study that compared treatment with DVTd to treatment with bortezomib, thalidomide and dexamethasone (VTd) in subjects with newly diagnosed multiple myeloma who are eligible for ASCT.

30) The method of claim 29, wherein the daratumumab-containing drug product label includes data that treatment with DVTd resulted in about 53% reduction in the risk of multiple myeloma progression or death when compared to treatment with VTd.

31) The method of claim 30, wherein the daratumumab-containing drug product label includes data that treatment with DVTd resulted in about 28.9% of subjects achieving the sCR or better, about 38.9% of subjects achieving the CR or better, and about 33.7% of subjects achieving a negative status for MRD, or any combination thereof.

32) The method of claim 29, wherein the daratumumab-containing drug product label includes a Kaplan-Meier curve of progression-free survival (PFS) comparing subjects having newly diagnosed multiple myeloma treated with DVTd to subjects having newly diagnosed multiple myeloma treated with VTd.

33) A method of treating a subject with newly diagnosed multiple myeloma, comprising:
a) providing a healthcare professional (HCP) daratumumab;
b) providing the HCP information that a combination therapy comprising daratumumab, bortezomib, melphalan and prednisone (DVMP) is demonstrated to increase a likelihood of achieving a stringent complete response (sCR) or better in subjects with newly diagnosed multiple myeloma; wherein performing the steps a) and b) results in the subject with newly diagnosed multiple myeloma to receive combination therapy comprising DVMP by the HCP or by self-administration as instructed by the HCP, thereby treating the subject having the newly diagnosed multiple myeloma.

34) The method of claim 33, wherein the information provided to the HCP is a daratumumab-containing drug product label that includes data from a phase 3 active-controlled study that compared treatment with DVMP to treatment with bortezomib, melphalan and prednisone (VMP) in subjects with newly diagnosed multiple myeloma.

35) A method of treating a subject with newly diagnosed multiple myeloma, comprising:
a) providing a healthcare professional (HCP) daratumumab;
b) providing the HCP information that a combination therapy comprising daratumumab, bortezomib and dexamethasone (DRd) is demonstrated to increase a likelihood of achieving a stringent complete response (sCR) or better in subjects with newly diagnosed multiple myeloma; wherein performing the steps a) and b) results in the subject with newly diagnosed multiple myeloma to receive combination therapy comprising DRd by the HCP or by self-administration as instructed by the HCP, thereby treating the subject having the newly diagnosed multiple myeloma.

36) The method of claim 33, wherein the information provided is a daratumumab-containing drug product label includes data from a phase 3 active-controlled study that compared treatment with DRd to treatment with lenalidomide and dexamethasone (Rd) in relapsed, refractory or relapsed and refractory multiple myeloma.

37) The method of any one of claim 1, 33 or 35, wherein daratumumab is a biosimilar of DARZALEX® brand of daratumumab.

38) The method of any one of claim 1, 33 or 35, wherein daratumumab comprises a heavy chain complementarity determining region 1 (HCDR1) of SEQ ID NO: 1, a HCDR2 of SEQ ID NO: 2, a HCDR3 of SEQ ID NO: 3, a light chain complementarity determining region 1 (LCDR1) of SEQ ID NO: 4, a LCDR2 of SEQ ID NO: 5 and a LCDR3 of SEQ ID NO: 6.

39) The method of any one of claim 1, 33 or 35, wherein dexamethasone can be substituted for a dexamethasone equivalent, wherein the dexamethasone equivalent is methylprednisolone, prednisolone, prednisone or betamethasone, or any combination thereof.


SEQUENCE LISTING (11 sequences)

SEQ ID NO: 1 - protein, 5 aa, Artificial Sequence
  HCDR1 of Daratumumab
SFAMS

SEQ ID NO: 2 - protein, 17 aa, Artificial Sequence
  HCDR2 of Daratumumab
AISGSGGGTYYADSVKG

SEQ ID NO: 3 - protein, 13 aa, Artificial Sequence
  HCDR3 of Daratumumab
DKILWFGEPVFDY

SEQ ID NO: 4 - protein, 11 aa, Artificial Sequence
  LCDR1 of Daratumumab
RASQSVSSYLA

SEQ ID NO: 5 - protein, 7 aa, Artificial Sequence
  LCDR2 of Daratumumab
DASNRAT

SEQ ID NO: 6 - protein, 10 aa, Artificial Sequence
  LCDR3 of Daratumumab
QQRSNWPPTF

SEQ ID NO: 7 - protein, 122 aa, Artificial Sequence [sequence omitted]

SEQ ID NO: 8 - protein, 107 aa, Artificial Sequence [sequence omitted]

SEQ ID NO: 9 - protein, 452 aa, Artificial Sequence
  HC of Daratumumab
EVQLLESGGGLVQPGGSLRLSCAVSGFTFNSFAMSWVRQAPGKGLEWVSAISGSGGGTYYADSVKGRFTISRDNSKNTLYLQMNSLRAEDTAVYFCAKDKILWFGEPVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, Artificial Sequence
  LC  of Daratumumab
EIVLTQSPATLSLSPGERATLSCRASQSVSSYLAWYQQKPGQAPRLLIYDASNRATGIPARFSGSGSGTDFTLTISSLEPEDFAVYYCQQRSNWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK